SEQUENCE LISTING (222 sequences)

SEQ ID NO: 1 - RNA, 17 nt, synthetic construct
  source 1..17: mol_type = other RNA
gtaagattaacacactg

SEQ ID NO: 2 - RNA, 17 nt, synthetic construct
  source 1..17: mol_type = other RNA
attaacaataagtaggg

SEQ ID NO: 3 - RNA, 17 nt, synthetic construct
  source 1..17: mol_type = other RNA
gctgagagacatattca

SEQ ID NO: 4 - RNA, 17 nt, synthetic construct
  source 1..17: mol_type = other RNA
tgagagagggtcaagtg

SEQ ID NO: 5 - RNA, 16 nt, synthetic construct
  source 1..16: mol_type = other RNA
cggaattatataggac

SEQ ID NO: 6 - RNA, 17 nt, synthetic construct
  source 1..17: mol_type = other RNA
gacattagtaaagcaga

SEQ ID NO: 7 - RNA, 17 nt, synthetic construct
  source 1..17: mol_type = other RNA
gactcagtaagaacacc

SEQ ID NO: 8 - RNA, 17 nt, synthetic construct
  source 1..17: mol_type = other RNA
taatgtcaagaatctca

SEQ ID NO: 9 - RNA, 17 nt, synthetic construct
  source 1..17: mol_type = other RNA
gttagaagtatttgttc

SEQ ID NO: 10 - RNA, 17 nt, synthetic construct
  source 1..17: mol_type = other RNA
agtaggagtaagttgat

SEQ ID NO: 11 - RNA, 17 nt, synthetic construct
  source 1..17: mol_type = other RNA
cgaggagaattagtctg

SEQ ID NO: 12 - RNA, 17 nt, synthetic construct
  source 1..17: mol_type = other RNA
gagttattagagtaagc

SEQ ID NO: 13 - RNA, 17 nt, synthetic construct
  source 1..17: mol_type = other RNA
tagcagcaatatcacca

SEQ ID NO: 14 - RNA, 17 nt, synthetic construct
  source 1..17: mol_type = other RNA
tagcagcaagattagca

SEQ ID NO: 15 - RNA, 17 nt, synthetic construct
  source 1..17: mol_type = other RNA
gactgagggaaggacat

SEQ ID NO: 16 - RNA, 16 nt, synthetic construct
  source 1..16: mol_type = other RNA
gtctgtagtaatgatt

SEQ ID NO: 17 - RNA, 17 nt, synthetic construct
  source 1..17: mol_type = other RNA
gcaaaggatcataaact

SEQ ID NO: 18 - RNA, 17 nt, synthetic construct
  source 1..17: mol_type = other RNA
acactcatcataaccta

SEQ ID NO: 19 - RNA, 17 nt, synthetic construct
  source 1..17: mol_type = other RNA
acagtagaaggttaggt

SEQ ID NO: 20 - RNA, 16 nt, synthetic construct
  source 1..16: mol_type = other RNA
actcgtaagacacatt

SEQ ID NO: 21 - RNA, 17 nt, synthetic construct
  source 1..17: mol_type = other RNA
agggaagaacataaggg

SEQ ID NO: 22 - RNA, 15 nt, synthetic construct
  source 1..15: mol_type = other RNA
agtgtaggtttaccg

SEQ ID NO: 23 - RNA, 17 nt, synthetic construct
  source 1..17: mol_type = other RNA
ataatcaattcactgtc

SEQ ID NO: 24 - RNA, 17 nt, synthetic construct
  source 1..17: mol_type = other RNA
atggaaggaggttggaa

SEQ ID NO: 25 - RNA, 17 nt, synthetic construct
  source 1..17: mol_type = other RNA
attgtttatcatactac

SEQ ID NO: 26 - RNA, 17 nt, synthetic construct
  source 1..17: mol_type = other RNA
cagacagataagatatg

SEQ ID NO: 27 - RNA, 17 nt, synthetic construct
  source 1..17: mol_type = other RNA
cagacagataagatatg

SEQ ID NO: 28 - RNA, 15 nt, synthetic construct
  source 1..15: mol_type = other RNA
ccttataacaggtcg

SEQ ID NO: 29 - RNA, 17 nt, synthetic construct
  source 1..17: mol_type = other RNA
cgaagacagtagaaggt

SEQ ID NO: 30 - RNA, 16 nt, synthetic construct
  source 1..16: mol_type = other RNA
cgtagatagcaaatta

SEQ ID NO: 31 - RNA, 17 nt, synthetic construct
  source 1..17: mol_type = other RNA
ctcaataggaaggtttg

SEQ ID NO: 32 - RNA, 17 nt, synthetic construct
  source 1..17: mol_type = other RNA
ctgtgagcaaatacaaa

SEQ ID NO: 33 - RNA, 17 nt, synthetic construct
  source 1..17: mol_type = other RNA
gagaaatagtagaaggg

SEQ ID NO: 34 - RNA, 17 nt, synthetic construct
  source 1..17: mol_type = other RNA
gatataggaaggatagg

SEQ ID NO: 35 - RNA, 16 nt, synthetic construct
  source 1..16: mol_type = other RNA
gcggtagtataggagc

SEQ ID NO: 36 - RNA, 17 nt, synthetic construct
  source 1..17: mol_type = other RNA
gttggagacagttataa

SEQ ID NO: 37 - RNA, 16 nt, synthetic construct
  source 1..16: mol_type = other RNA
gtttgaaggttcacgt

SEQ ID NO: 38 - RNA, 17 nt, synthetic construct
  source 1..17: mol_type = other RNA
tactcaataggaaggtt

SEQ ID NO: 39 - RNA, 17 nt, synthetic construct
  source 1..17: mol_type = other RNA
tcacttacatattacac

SEQ ID NO: 40 - RNA, 16 nt, synthetic construct
  source 1..16: mol_type = other RNA
tcgagtacagttggtt

SEQ ID NO: 41 - RNA, 17 nt, synthetic construct
  source 1..17: mol_type = other RNA
tgggagtaaggaacacg

SEQ ID NO: 42 - RNA, 17 nt, synthetic construct
  source 1..17: mol_type = other RNA
tgtagagaagggaggta

SEQ ID NO: 43 - RNA, 16 nt, synthetic construct
  source 1..16: mol_type = other RNA
ttgataacacacacgg

SEQ ID NO: 44 - RNA, 15 nt, synthetic construct
  source 1..15: mol_type = other RNA
gttcgtttagagaac

SEQ ID NO: 45 - RNA, 16 nt, synthetic construct
  source 1..16: mol_type = other RNA
cgtttagagaacagat

SEQ ID NO: 46 - RNA, 16 nt, synthetic construct
  source 1..16: mol_type = other RNA
gtttagagaacagatc

SEQ ID NO: 47 - RNA, 17 nt, synthetic construct
  source 1..17: mol_type = other RNA
gagaacagatctacaag

SEQ ID NO: 48 - RNA, 15 nt, synthetic construct
  source 1..15: mol_type = other RNA
gaacagatctacaag

SEQ ID NO: 49 - RNA, 17 nt, synthetic construct
  source 1..17: mol_type = other RNA
cagatctacaagagatc

SEQ ID NO: 50 - RNA, 16 nt, synthetic construct
  source 1..16: mol_type = other RNA
gatctacaagagatcg

SEQ ID NO: 51 - RNA, 16 nt, synthetic construct
  source 1..16: mol_type = other RNA
caagagatcgaaagtt

SEQ ID NO: 52 - RNA, 15 nt, synthetic construct
  source 1..15: mol_type = other RNA
gatctacaagagatc

SEQ ID NO: 53 - RNA, 16 nt, synthetic construct
  source 1..16: mol_type = other RNA
gagatcgaaagttggt

SEQ ID NO: 54 - RNA, 16 nt, synthetic construct
  source 1..16: mol_type = other RNA
gaaagttggttggttt

SEQ ID NO: 55 - RNA, 16 nt, synthetic construct
  source 1..16: mol_type = other RNA
gttggtttgttacctg

SEQ ID NO: 56 - RNA, 16 nt, synthetic construct
  source 1..16: mol_type = other RNA
ggttggtttgttacct

SEQ ID NO: 57 - RNA, 16 nt, synthetic construct
  source 1..16: mol_type = other RNA
gtttgttacctgggaa

SEQ ID NO: 58 - RNA, 15 nt, synthetic construct
  source 1..15: mol_type = other RNA
gttacctgggaaggt

SEQ ID NO: 59 - RNA, 16 nt, synthetic construct
  source 1..16: mol_type = other RNA
ctgtatacgacatcag

SEQ ID NO: 60 - RNA, 16 nt, synthetic construct
  source 1..16: mol_type = other RNA
tgtagatgtcaaaagc

SEQ ID NO: 61 - RNA, 16 nt, synthetic construct
  source 1..16: mol_type = other RNA
cttcgtccttttcttg

SEQ ID NO: 62 - RNA, 16 nt, synthetic construct
  source 1..16: mol_type = other RNA
gaagcgacaacaatta

SEQ ID NO: 63 - RNA, 16 nt, synthetic construct
  source 1..16: mol_type = other RNA
gtttttaggaatttag

SEQ ID NO: 64 - RNA, 16 nt, synthetic construct
  source 1..16: mol_type = other RNA
caaaaccagctacttt

SEQ ID NO: 65 - RNA, 16 nt, synthetic construct
  source 1..16: mol_type = other RNA
atcattgtagatgtca

SEQ ID NO: 66 - RNA, 16 nt, synthetic construct
  source 1..16: mol_type = other RNA
aaagccctgtatacga

SEQ ID NO: 67 - RNA, 16 nt, synthetic construct
  source 1..16: mol_type = other RNA
catcagtactagtgcc

SEQ ID NO: 68 - RNA, 16 nt, synthetic construct
  source 1..16: mol_type = other RNA
tgtgccgcacggtgta

SEQ ID NO: 69 - RNA, 16 nt, synthetic construct
  source 1..16: mol_type = other RNA
agacgggctgcactta

SEQ ID NO: 70 - RNA, 16 nt, synthetic construct
  source 1..16: mol_type = other RNA
caccgcaaacccgttt

SEQ ID NO: 71 - RNA, 16 nt, synthetic construct
  source 1..16: mol_type = other RNA
aaaaacgattgtgcat

SEQ ID NO: 72 - RNA, 16 nt, synthetic construct
  source 1..16: mol_type = other RNA
cagctgactgaagcat

SEQ ID NO: 73 - RNA, 16 nt, synthetic construct
  source 1..16: mol_type = other RNA
gggttcgcggagttga

SEQ ID NO: 74 - RNA, 16 nt, synthetic construct
  source 1..16: mol_type = other RNA
tcacaactacagccat

SEQ ID NO: 75 - RNA, 16 nt, synthetic construct
  source 1..16: mol_type = other RNA
aacctttccacatacc

SEQ ID NO: 76 - RNA, 16 nt, synthetic construct
  source 1..16: mol_type = other RNA
gcagacggtacagact

SEQ ID NO: 77 - RNA, 14 nt, synthetic construct
  source 1..14: mol_type = other RNA
gtgtttttaagtgt

SEQ ID NO: 78 - RNA, 14 nt, synthetic construct
  source 1..14: mol_type = other RNA
aaaacccacagggt

SEQ ID NO: 79 - RNA, 16 nt, synthetic construct
  source 1..16: mol_type = other RNA
ccttttcttggaagcg

SEQ ID NO: 80 - RNA, 16 nt, synthetic construct
  source 1..16: mol_type = other RNA
acaacaattagttttt

SEQ ID NO: 81 - RNA, 16 nt, synthetic construct
  source 1..16: mol_type = other RNA
aggaatttagcaaaac

SEQ ID NO: 82 - RNA, 16 nt, synthetic construct
  source 1..16: mol_type = other RNA
cagctactttatcatt

SEQ ID NO: 83 - RNA, 16 nt, synthetic construct
  source 1..16: mol_type = other RNA
gtagatgtcaaaagcc

SEQ ID NO: 84 - RNA, 16 nt, synthetic construct
  source 1..16: mol_type = other RNA
tactagtgcctgtgcc

SEQ ID NO: 85 - RNA, 16 nt, synthetic construct
  source 1..16: mol_type = other RNA
gcacggtgtaagacgg

SEQ ID NO: 86 - RNA, 16 nt, synthetic construct
  source 1..16: mol_type = other RNA
gctgcacttacaccgc

SEQ ID NO: 87 - RNA, 16 nt, synthetic construct
  source 1..16: mol_type = other RNA
aaacccgtttaaaaac

SEQ ID NO: 88 - RNA, 16 nt, synthetic construct
  source 1..16: mol_type = other RNA
gattgtgcatcagctg

SEQ ID NO: 89 - RNA, 16 nt, synthetic construct
  source 1..16: mol_type = other RNA
actgaagcatgggttc

SEQ ID NO: 90 - RNA, 16 nt, synthetic construct
  source 1..16: mol_type = other RNA
gcggagttgatcacaa

SEQ ID NO: 91 - RNA, 16 nt, synthetic construct
  source 1..16: mol_type = other RNA
ctacagccataacctt

SEQ ID NO: 92 - RNA, 16 nt, synthetic construct
  source 1..16: mol_type = other RNA
tccacataccgcagac

SEQ ID NO: 93 - RNA, 16 nt, synthetic construct
  source 1..16: mol_type = other RNA
ggtacagactgtgttt

SEQ ID NO: 94 - RNA, 17 nt, synthetic construct
  source 1..17: mol_type = other RNA
ttaagtgtaaaacccac

SEQ ID NO: 95 - RNA, 16 nt, synthetic construct
  source 1..16: mol_type = other RNA
aacgattgtgcatcag

SEQ ID NO: 96 - RNA, 16 nt, synthetic construct
  source 1..16: mol_type = other RNA
ctgactgaagcatggg

SEQ ID NO: 97 - RNA, 17 nt, Severe acute respiratory syndrome-related coronavirus
  source 1..17: mol_type = genomic RNA
cagtgtgttaatcttac

SEQ ID NO: 98 - RNA, 17 nt, Severe acute respiratory syndrome-related coronavirus
  source 1..17: mol_type = genomic RNA
ccctacttattgttaat

SEQ ID NO: 99 - RNA, 17 nt, Severe acute respiratory syndrome-related coronavirus
  source 1..17: mol_type = genomic RNA
tgaatatgtctctcagc

SEQ ID NO: 100 - RNA, 17 nt, Severe acute respiratory syndrome-related coronavirus
  source 1..17: mol_type = genomic RNA
cacttgaccctctctca

SEQ ID NO: 101 - RNA, 16 nt, Severe acute respiratory syndrome-related coronavirus
  source 1..16: mol_type = genomic RNA
gtcctatataattccg

SEQ ID NO: 102 - RNA, 17 nt, Severe acute respiratory syndrome-related coronavirus
  source 1..17: mol_type = genomic RNA
tctgctttactaatgtc

SEQ ID NO: 103 - RNA, 17 nt, Severe acute respiratory syndrome-related coronavirus
  source 1..17: mol_type = genomic RNA
ggtgttcttactgagtc

SEQ ID NO: 104 - RNA, 17 nt, Severe acute respiratory syndrome-related coronavirus
  source 1..17: mol_type = genomic RNA
tgagattcttgacatta

SEQ ID NO: 105 - RNA, 17 nt, Severe acute respiratory syndrome-related coronavirus
  source 1..17: mol_type = genomic RNA
gaacaaatacttctaac

SEQ ID NO: 106 - RNA, 17 nt, Severe acute respiratory syndrome-related coronavirus
  source 1..17: mol_type = genomic RNA
atcaacttactcctact

SEQ ID NO: 107 - RNA, 17 nt, Severe acute respiratory syndrome-related coronavirus
  source 1..17: mol_type = genomic RNA
cagactaattctcctcg

SEQ ID NO: 108 - RNA, 17 nt, Severe acute respiratory syndrome-related coronavirus
  source 1..17: mol_type = genomic RNA
gcttactctaataactc

SEQ ID NO: 109 - RNA, 17 nt, Severe acute respiratory syndrome-related coronavirus
  source 1..17: mol_type = genomic RNA
tggtgatattgctgcta

SEQ ID NO: 110 - RNA, 17 nt, Severe acute respiratory syndrome-related coronavirus
  source 1..17: mol_type = genomic RNA
tgctaatcttgctgcta

SEQ ID NO: 111 - RNA, 17 nt, Severe acute respiratory syndrome-related coronavirus
  source 1..17: mol_type = genomic RNA
atgtccttccctcagtc

SEQ ID NO: 112 - RNA, 16 nt, Severe acute respiratory syndrome-related coronavirus
  source 1..16: mol_type = genomic RNA
aatcattactacagac

SEQ ID NO: 113 - RNA, 17 nt, Severe acute respiratory syndrome-related coronavirus
  source 1..17: mol_type = genomic RNA
agtttatgatcctttgc

SEQ ID NO: 114 - RNA, 17 nt, Homo sapiens
  source 1..17: mol_type = genomic RNA
taggttatgatgagtgt

SEQ ID NO: 115 - RNA, 17 nt, Homo sapiens
  source 1..17: mol_type = genomic RNA
acctaaccttctactgt

SEQ ID NO: 116 - RNA, 16 nt, Homo sapiens
  source 1..16: mol_type = genomic RNA
aatgtgtcttacgagt

SEQ ID NO: 117 - RNA, 17 nt, Homo sapiens
  source 1..17: mol_type = genomic RNA
cccttatgttcttccct

SEQ ID NO: 118 - RNA, 15 nt, Homo sapiens
  source 1..15: mol_type = genomic RNA
cggtaaacctacact

SEQ ID NO: 119 - RNA, 17 nt, Homo sapiens
  source 1..17: mol_type = genomic RNA
gacagtgaattgattat

SEQ ID NO: 120 - RNA, 17 nt, Homo sapiens
  source 1..17: mol_type = genomic RNA
ttccaacctccttccat

SEQ ID NO: 121 - RNA, 17 nt, Homo sapiens
  source 1..17: mol_type = genomic RNA
gtagtatgataaacaat

SEQ ID NO: 122 - RNA, 17 nt, Homo sapiens
  source 1..17: mol_type = genomic RNA
catatcttatctgtctg

SEQ ID NO: 123 - RNA, 17 nt, Homo sapiens
  source 1..17: mol_type = genomic RNA
gagtttgtgtaatgtgg

SEQ ID NO: 124 - RNA, 15 nt, Homo sapiens
  source 1..15: mol_type = genomic RNA
cgacctgttataagg

SEQ ID NO: 125 - RNA, 17 nt, Homo sapiens
  source 1..17: mol_type = genomic RNA
accttctactgtcttcg

SEQ ID NO: 126 - RNA, 16 nt, Homo sapiens
  source 1..16: mol_type = genomic RNA
taatttgctatctacg

SEQ ID NO: 127 - RNA, 17 nt, Homo sapiens
  source 1..17: mol_type = genomic RNA
caaaccttcctattgag

SEQ ID NO: 128 - RNA, 17 nt, Homo sapiens
  source 1..17: mol_type = genomic RNA
tttgtatttgctcacag

SEQ ID NO: 129 - RNA, 17 nt, Homo sapiens
  source 1..17: mol_type = genomic RNA
cccttctactatttctc

SEQ ID NO: 130 - RNA, 17 nt, Homo sapiens
  source 1..17: mol_type = genomic RNA
cctatccttcctatatc

SEQ ID NO: 131 - RNA, 16 nt, Homo sapiens
  source 1..16: mol_type = genomic RNA
gctcctatactaccgc

SEQ ID NO: 132 - RNA, 17 nt, Homo sapiens
  source 1..17: mol_type = mRNA
ttataactgtctccaac

SEQ ID NO: 133 - RNA, 16 nt, Homo sapiens
  source 1..16: mol_type = genomic RNA
acgtgaaccttcaaac

SEQ ID NO: 134 - RNA, 17 nt, Homo sapiens
  source 1..17: mol_type = genomic RNA
aaccttcctattgagta

SEQ ID NO: 135 - RNA, 17 nt, Homo sapiens
  source 1..17: mol_type = genomic RNA
gtgtaatatgtaagtga

SEQ ID NO: 136 - RNA, 16 nt, Homo sapiens
  source 1..16: mol_type = genomic RNA
aaccaactgtactcga

SEQ ID NO: 137 - RNA, 17 nt, Homo sapiens
  source 1..17: mol_type = genomic RNA
cgtgttccttactccca

SEQ ID NO: 138 - RNA, 17 nt, Homo sapiens
  source 1..17: mol_type = genomic RNA
tacctcccttctctaca

SEQ ID NO: 139 - RNA, 16 nt, Homo sapiens
  source 1..16: mol_type = genomic RNA
ccgtgtgtgttatcaa

SEQ ID NO: 140 - RNA, 15 nt, Severe acute respiratory syndrome-related coronavirus
  source 1..15: mol_type = genomic RNA
gttctctaaacgaac

SEQ ID NO: 141 - RNA, 16 nt, Severe acute respiratory syndrome-related coronavirus
  source 1..16: mol_type = genomic RNA
atctgttctctaaacg

SEQ ID NO: 142 - RNA, 16 nt, Severe acute respiratory syndrome-related coronavirus
  source 1..16: mol_type = genomic RNA
gatctgttctctaaac

SEQ ID NO: 143 - RNA, 17 nt, Severe acute respiratory syndrome-related coronavirus
  source 1..17: mol_type = genomic RNA
cttgtagatctgttctc

SEQ ID NO: 144 - RNA, 15 nt, Severe acute respiratory syndrome-related coronavirus
  source 1..15: mol_type = genomic RNA
cttgtagatctgttc

SEQ ID NO: 145 - RNA, 17 nt, Homo sapiens
  source 1..17: mol_type = genomic RNA
gatctcttgtagatctg

SEQ ID NO: 146 - RNA, 16 nt, Severe acute respiratory syndrome-related coronavirus
  source 1..16: mol_type = genomic RNA
cgatctcttgtagatc

SEQ ID NO: 147 - RNA, 16 nt, Severe acute respiratory syndrome-related coronavirus
  source 1..16: mol_type = genomic RNA
aactttcgatctcttg

SEQ ID NO: 148 - RNA, 15 nt, Severe acute respiratory syndrome-related coronavirus
  source 1..15: mol_type = genomic RNA
gatctcttgtagatc

SEQ ID NO: 149 - RNA, 16 nt, Severe acute respiratory syndrome-related coronavirus
  source 1..16: mol_type = genomic RNA
accaactttcgatctc

SEQ ID NO: 150 - RNA, 16 nt, Severe acute respiratory syndrome-related coronavirus
  source 1..16: mol_type = genomic RNA
aaaccaaccaactttc

SEQ ID NO: 151 - RNA, 16 nt, Severe acute respiratory syndrome-related coronavirus
  source 1..16: mol_type = genomic RNA
caggtaacaaaccaac

SEQ ID NO: 152 - RNA, 16 nt, Severe acute respiratory syndrome-related coronavirus
  source 1..16: mol_type = genomic RNA
aggtaacaaaccaacc

SEQ ID NO: 153 - RNA, 16 nt, Severe acute respiratory syndrome-related coronavirus
  source 1..16: mol_type = genomic RNA
ttcccaggtaacaaac

SEQ ID NO: 154 - RNA, 15 nt, Severe acute respiratory syndrome-related coronavirus
  source 1..15: mol_type = genomic RNA
accttcccaggtaac

SEQ ID NO: 155 - RNA, 16 nt, Severe acute respiratory syndrome-related coronavirus
  source 1..16: mol_type = genomic RNA
ctgatgtcgtatacag

SEQ ID NO: 156 - RNA, 16 nt, Severe acute respiratory syndrome-related coronavirus
  source 1..16: mol_type = genomic RNA
gcttttgacatctaca

SEQ ID NO: 157 - RNA, 16 nt, Severe acute respiratory syndrome-related coronavirus
  source 1..16: mol_type = genomic RNA
caagaaaaggacgaag

SEQ ID NO: 158 - RNA, 16 nt, Severe acute respiratory syndrome-related coronavirus
  source 1..16: mol_type = genomic RNA
taattgttgtcgcttc

SEQ ID NO: 159 - RNA, 16 nt, Severe acute respiratory syndrome-related coronavirus
  source 1..16: mol_type = genomic RNA
ctaaattcctaaaaac

SEQ ID NO: 160 - RNA, 16 nt, Severe acute respiratory syndrome-related coronavirus
  source 1..16: mol_type = genomic RNA
aaagtagctggttttg

SEQ ID NO: 161 - RNA, 15 nt, Severe acute respiratory syndrome-related coronavirus
  source 1..15: mol_type = genomic RNA
tgacatctacaatat

SEQ ID NO: 162 - RNA, 16 nt, Severe acute respiratory syndrome-related coronavirus
  source 1..16: mol_type = genomic RNA
tcgtatacagggcttt

SEQ ID NO: 163 - RNA, 16 nt, Severe acute respiratory syndrome-related coronavirus
  source 1..16: mol_type = genomic RNA
ggcactagtactgatg

SEQ ID NO: 164 - RNA, 16 nt, Severe acute respiratory syndrome-related coronavirus
  source 1..16: mol_type = genomic RNA
tacaccgtgcggcaca

SEQ ID NO: 165 - RNA, 16 nt, Severe acute respiratory syndrome-related coronavirus
  source 1..16: mol_type = genomic RNA
taagtgcagcccgtct

SEQ ID NO: 166 - RNA, 16 nt, Severe acute respiratory syndrome-related coronavirus
  source 1..16: mol_type = genomic RNA
aaacgggtttgcggtg

SEQ ID NO: 167 - RNA, 16 nt, Severe acute respiratory syndrome-related coronavirus
  source 1..16: mol_type = genomic RNA
atgcacaatcgttttt

SEQ ID NO: 168 - RNA, 16 nt, Severe acute respiratory syndrome-related coronavirus
  source 1..16: mol_type = genomic RNA
atgcttcagtcagctg

SEQ ID NO: 169 - RNA, 16 nt, Severe acute respiratory syndrome-related coronavirus
  source 1..16: mol_type = genomic RNA
tcaactccgcgaaccc

SEQ ID NO: 170 - RNA, 16 nt, Severe acute respiratory syndrome-related coronavirus
  source 1..16: mol_type = genomic RNA
atggctgtagttgtga

SEQ ID NO: 171 - RNA, 16 nt, Severe acute respiratory syndrome-related coronavirus
  source 1..16: mol_type = genomic RNA
ggtatgtggaaaggtt

SEQ ID NO: 172 - RNA, 16 nt, Severe acute respiratory syndrome-related coronavirus
  source 1..16: mol_type = genomic RNA
agtctgtaccgtctgc

SEQ ID NO: 173 - RNA, 14 nt, Severe acute respiratory syndrome-related coronavirus
  source 1..14: mol_type = genomic RNA
acacttaaaaacac

SEQ ID NO: 174 - RNA, 14 nt, Severe acute respiratory syndrome-related coronavirus
  source 1..14: mol_type = genomic RNA
accctgtgggtttt

SEQ ID NO: 175 - RNA, 16 nt, Severe acute respiratory syndrome-related coronavirus
  source 1..16: mol_type = genomic RNA
cgcttccaagaaaagg

SEQ ID NO: 176 - RNA, 16 nt, Severe acute respiratory syndrome-related coronavirus
  source 1..16: mol_type = genomic RNA
aaaaactaattgttgt

SEQ ID NO: 177 - RNA, 16 nt, Severe acute respiratory syndrome-related coronavirus
  source 1..16: mol_type = genomic RNA
gttttgctaaattcct

SEQ ID NO: 178 - RNA, 16 nt, Severe acute respiratory syndrome-related coronavirus
  source 1..16: mol_type = genomic RNA
aatgataaagtagctg

SEQ ID NO: 179 - RNA, 16 nt, Severe acute respiratory syndrome-related coronavirus
  source 1..16: mol_type = genomic RNA
ggcttttgacatctac

SEQ ID NO: 180 - RNA, 16 nt, Severe acute respiratory syndrome-related coronavirus
  source 1..16: mol_type = genomic RNA
ggcacaggcactagta

SEQ ID NO: 181 - RNA, 16 nt, Severe acute respiratory syndrome-related coronavirus
  source 1..16: mol_type = genomic RNA
ccgtcttacaccgtgc

SEQ ID NO: 182 - RNA, 16 nt, Severe acute respiratory syndrome-related coronavirus
  source 1..16: mol_type = genomic RNA
gcggtgtaagtgcagc

SEQ ID NO: 183 - RNA, 16 nt, Severe acute respiratory syndrome-related coronavirus
  source 1..16: mol_type = genomic RNA
gtttttaaacgggttt

SEQ ID NO: 184 - RNA, 16 nt, Severe acute respiratory syndrome-related coronavirus
  source 1..16: mol_type = genomic RNA
cagctgatgcacaatc

SEQ ID NO: 185 - RNA, 16 nt, Severe acute respiratory syndrome-related coronavirus
  source 1..16: mol_type = genomic RNA
gaacccatgcttcagt

SEQ ID NO: 186 - RNA, 16 nt, Severe acute respiratory syndrome-related coronavirus
  source 1..16: mol_type = genomic RNA
ttgtgatcaactccgc

SEQ ID NO: 187 - RNA, 16 nt, Severe acute respiratory syndrome-related coronavirus
  source 1..16: mol_type = genomic RNA
aaggttatggctgtag

SEQ ID NO: 188 - RNA, 16 nt, Severe acute respiratory syndrome-related coronavirus
  source 1..16: mol_type = genomic RNA
gtctgcggtatgtgga

SEQ ID NO: 189 - RNA, 16 nt, Severe acute respiratory syndrome-related coronavirus
  source 1..16: mol_type = genomic RNA
aaacacagtctgtacc

SEQ ID NO: 190 - RNA, 17 nt, Severe acute respiratory syndrome-related coronavirus
  source 1..17: mol_type = genomic RNA
gtgggttttacacttaa

SEQ ID NO: 191 - RNA, 15 nt, Severe acute respiratory syndrome-related coronavirus
  source 1..15: mol_type = genomic RNA
tgcttcagtcagctg

SEQ ID NO: 192 - RNA, 13 nt, Severe acute respiratory syndrome-related coronavirus
  source 1..13: mol_type = genomic RNA
ctccgcgaaccca

SEQ ID NO: 193 - RNA, 16 nt, Severe acute respiratory syndrome-related coronavirus
  source 1..16: mol_type = genomic RNA
tttaaacgggtttgcg

SEQ ID NO: 194 - RNA, 16 nt, Severe acute respiratory syndrome-related coronavirus
  source 1..16: mol_type = genomic RNA
ctgatgcacaatcgtt

SEQ ID NO: 195 - RNA, 16 nt, Severe acute respiratory syndrome-related coronavirus
  source 1..16: mol_type = genomic RNA
cccatgcttcagtcag

SEQ ID NO: 196 - RNA, 16 nt, Severe acute respiratory syndrome-related coronavirus
  source 1..16: mol_type = genomic RNA
ggtttcgtccgtgttg

SEQ ID NO: 197 - RNA, 16 nt, Severe acute respiratory syndrome-related coronavirus
  source 1..16: mol_type = genomic RNA
cgttgacaggacacga

SEQ ID NO: 198 - RNA, 17 nt, Severe acute respiratory syndrome-related coronavirus
  source 1..17: mol_type = genomic RNA
taataactaattactgt

SEQ ID NO: 199 - RNA, 16 nt, Severe acute respiratory syndrome-related coronavirus
  source 1..16: mol_type = genomic RNA
gatggagagccttgtc

SEQ ID NO: 200 - RNA, 15 nt, Severe acute respiratory syndrome-related coronavirus
  source 1..15: mol_type = genomic RNA
ggtgtgaccgaaagg

SEQ ID NO: 201 - RNA, 16 nt, Severe acute respiratory syndrome-related coronavirus
  source 1..16: mol_type = genomic RNA
atctaggtttcgtccg

SEQ ID NO: 202 - RNA, 17 nt, Severe acute respiratory syndrome-related coronavirus
  source 1..17: mol_type = genomic RNA
cagccgatcatcagcac

SEQ ID NO: 203 - RNA, 15 nt, Severe acute respiratory syndrome-related coronavirus
  source 1..15: mol_type = genomic RNA
ctgcaggctgcttac

SEQ ID NO: 204 - RNA, 16 nt, Severe acute respiratory syndrome-related coronavirus
  source 1..16: mol_type = genomic RNA
gtaactcgtctatctt

SEQ ID NO: 205 - RNA, 15 nt, Severe acute respiratory syndrome-related coronavirus
  source 1..15: mol_type = genomic RNA
ctcacgcagtataat

SEQ ID NO: 206 - RNA, 16 nt, Severe acute respiratory syndrome-related coronavirus
  source 1..16: mol_type = genomic RNA
ctgcatgcttagtgca

SEQ ID NO: 207 - RNA, 17 nt, Severe acute respiratory syndrome-related coronavirus
  source 1..17: mol_type = genomic RNA
gtgtggctgtcactcgg

SEQ ID NO: 208 - RNA, 16 nt, Severe acute respiratory syndrome-related coronavirus
  source 1..16: mol_type = genomic RNA
acgaactttaaaatct

SEQ ID NO: 209 - RNA, 17 nt, Severe acute respiratory syndrome-related coronavirus
  source 1..17: mol_type = genomic RNA
gtagatctgttctctaa

SEQ ID NO: 210 - RNA, 16 nt, Severe acute respiratory syndrome-related coronavirus
  source 1..16: mol_type = genomic RNA
caactttcgatctctt

SEQ ID NO: 211 - RNA, 16 nt, Severe acute respiratory syndrome-related coronavirus
  source 1..16: mol_type = genomic RNA
aaggtttataccttcc

SEQ ID NO: 212 - DNA, 20 bp, synthetic construct
  source 1..20: mol_type = other DNA
gaccccaaaatcagcgaaat

SEQ ID NO: 213 - DNA, 24 bp, synthetic construct
  source 1..24: mol_type = other DNA
tctggttactgccagttgaatctg

SEQ ID NO: 214 - DNA, 20 bp, Severe acute respiratory syndrome-related coronavirus
  source 1..20: mol_type = other DNA
gtccttccctcagtcagcac

SEQ ID NO: 215 - DNA, 20 bp, synthetic construct
  source 1..20: mol_type = other DNA
atggcaggagcagttgtgaa

SEQ ID NO: 216 - DNA, 20 bp, Severe acute respiratory syndrome-related coronavirus
  source 1..20: mol_type = other DNA
gtaacccgttgaaccccatt

SEQ ID NO: 217 - DNA, 20 bp, synthetic construct
  source 1..20: mol_type = other DNA
ccatccaatcggtagtagcg

SEQ ID NO: 218 - DNA, 20 bp, synthetic construct
  source 1..20: mol_type = other DNA
gcaattattccccatgaacg

SEQ ID NO: 219 - DNA, 20 bp, synthetic construct
  source 1..20: mol_type = other DNA
ggcctcactaaaccatccaa

SEQ ID NO: 220 - DNA, 48 bp, synthetic construct
  source 1..48: mol_type = other DNA
taatacgactcactatagggattaaaggtttataccttcccaggtaac

SEQ ID NO: 221 - DNA, 20 bp, synthetic construct
  source 1..20: mol_type = other DNA
tcgttgaaaccagggacaag

SEQ ID NO: 222 - DNA, 24 bp, synthetic construct
  source 1..24: mol_type = other DNA
gggattaaaggtttataccttccc

(19) United States
(12) Patent Application Publication
(10) Pub. No.: US 2024/0102015 A1
(43) Pub. Date: Mar. 28, 2024

(54) Antisense Oligonucleotides Targeting SARS-CoV-2

(71) Applicants: The Regents of the University of California, Oakland, CA (US); Aalborg University, Aalborg (DK)

(72) Inventors: Anders Michael Naar, Berkeley, CA (US); Chi Zhu, Berkeley, CA (US); Sakari Kauppinen, Copenhagen (DK); Kasper Thystrup Karstensen, Copenhagen (DK)

(73) Assignees: The Regents of the University of California, Oakland, CA (US); Aalborg University, Aalborg (DK)

(21) Appl. No.: 18/157,063

(22) Filed: Jan. 19, 2023

Related U.S. Application Data
(63) Continuation of application No. PCT/US21/41503, filed on Jul. 13, 2021.
(60) Provisional application No. 63/147,225, filed on Feb. 8, 2021.
(60) Provisional application No. 63/062,417, filed on Aug. 6, 2020.

(51) Int. Cl. C12N 15/113; A61K 9/00; A61P 31/14

(52) U.S. Cl. CPC C12N 15/1131; A61K 9/0043; A61P 31/14; C12N 2310/11; C12N 2310/321; C12N 2310/3233

(57) ABSTRACT

COVID-19 is treated with antisense oligonucleotides (ASOs) targeting SARS-CoV-2 viral RNAs or human ACE2 RNA.

### REFERENCE TO A SEQUENCE LISTING

A Sequence Listing in XML format is incorporated by reference into the specification. The name of the XML file containing the Sequence Listing is B21-006-3US.xml. The XML file is 198,848 bytes and was created and submitted electronically via EFS-Web on Jan. 19, 2023.

### INTRODUCTION

The global coronavirus disease 19 (COVID-19) pandemic is caused by the highly pathogenic novel human SARS-coronavirus 2 (SARS-CoV-2)⁵. By contrast with previous outbreaks of related beta-coronaviruses (severe acute respiratory syndrome (SARS) and Middle East respiratory syndrome (MERS)), SARS-CoV-2 infection causes lower mortality rate, however, the virus has a higher human-to-human transmission rate⁶, facilitating rapid spread across the world. As of May 2021, there are more than 150 million confirmed positive cases and in excess of 3 million reported deaths (WHO; www.who.int). Although vaccines are markedly slowing the increase in positive cases and deaths in a few developed countries, most nations, especially in the developing world, do not have readily accessible vaccines⁷, and there is aversion among segments of the global population to vaccination⁷. Moreover, mutated variant strains of SARS-CoV-2 that evade immunity in response to previous infection or vaccination are rapidly emerging, and are causing new local outbreaks, frequently amongst younger individuals and with more severe disease^{1,8}. Indeed, more than 80 variants have been identified to date, all with mutations in the Spike protein⁹, which SARS-CoV-2 utilizes for binding to the host cell-surface receptor angiotensin-converting enzyme 2 (ACE2) during host cell entry¹⁰. Several “variants of concern”, which harbor mutations in Spike that facilitate immune evasion and, in some cases, partial vaccine and monoclonal antibody therapeutics resistance, are spreading rapidly world-wide. These include B.1.1.7 (UK variant), B.1.351 (South African/SA variant), P.1 (Brazilian variant), B.1.427/B.1.429 (CA variants), and B.1.617 (Indian variant), which are all more contagious and may in some cases lead to more severe disease¹¹. The continuous evolution of the virus thus poses a daunting challenge to achieving “herd immunity”. Traditional drug screening and vaccine development is time intensive, and may not be able to match the speed of emerging drug- or vaccine-resistant SARS-CoV-2 strains. There is clearly an urgent need for alternative approaches, including the rapid development of therapeutics that are active against all variants of concern.

### SUMMARY OF THE INVENTION

The invention provides therapeutic and prophylactic methods and compositions to treat COVID-19 with antisense oligonucleotides (ASOs) targeting SARS-CoV-2 viral RNAs and/or human ACE2, either alone or in combination. Our methods and compositions may be used to prevent the entry of SARS-CoV-2 and production of infectious viral particles in COVID-19 patients with acute respiratory disease, for example, by using inhaled aerosolized ASOs, or by once-weekly or monthly subcutaneous injection. Advantages of LNA ASOs over standard ASO technologies include higher affinity for the target RNA molecules, translating into high on-target specificity, lower dosing, tolerability and lower or no side effects. In addition, LNA ASOs minimize the need for medicinal chemistry optimization and significantly shorten the development timeline to Initial New Drug (IND) submission with the FDA.

In an aspect, the invention provides a method of treating COVID-19 comprising administering to a person in need thereof an antisense oligonucleotide (ASO) targeting SARS-CoV-2 viral RNAs or human ACE2 mRNA.

In embodiments:
the oligonucleotide comprises a modification selected from: locked nucleic acid (LNA) modification; 2′ O-methoxyethyl (MOE) modification; 2′ O-methyl (OME) modification; morpholino phosphorodiamidate (MP) modification; and phosphorodiamidate morpholino oligomers (PMOs) modification;
the oligonucleotide comprises a locked nucleic acid (LNA) modified antisense oligonucleotide (ASO), the LNA modification comprising a methylene bridge connecting 2′-oxygen and 4′-carbon of ribose;
the administering step comprises delivering the oligonucleotide by injection or inhalation;
the administering step comprises delivering the oligonucleotide by nasal administration.
comprising administering to the person a combination of oligonucleotides targeting SARS-CoV-2 viral RNAs and human ACE2 RNA;
further comprising detecting a resultant diminution in the person of SARS-CoV-2 presence, expression or activity;
the oligonucleotide hybridizes to a predetermined target site of a SARS-CoV-2 viral RNA region: 5′ leader, ORF1a, ORF1b, S, ORF3a, E, M, ORF6, ORF7, ORFS, N, ORF10, or 3′ UTR;
the oligonucleotide hybridizes to a predetermined target site of a SARS-CoV-2 viral RNA region: 5′ leader sequence, Spike coding sequence, 5′ untranslated region (UTR) of ORF1a/b, or frameshift element (FSE) region ORF1a/b;
the oligonucleotide hybridizes to a predetermined target site of a SARS-CoV-2 viral RNA region: 5′ leader sequence;
the oligonucleotide(s) comprises sufficient complementarity with a target site of a Table herein to effect hybridization thereto;
the oligonucleotide comprises a sequence complementary with a target site of a Table herein;
the oligonucleotide comprises a sequence of an antisense oligonucleotide (ASO) of a Table herein;
the oligonucleotide comprises a sequence and locked nucleic acid (LNA) pattern of an antisense oligonucleotide (ASO) of a Table herein;
the oligonucleotide hybridizes to a predetermined target site of a SARS-CoV-2 viral RNA region:

| | | | |
|---|---|---|---|
| ASO | 5′ leader sequence | ASO | 5′UTR regions of ORF1a |
| 5′-ASO#11 | AAACCAACCAACUUUC | U-ASO#05 | GGUUUCGUCCGUGUUG |
| | (SEQ ID NO: 150) | | (SEQ ID NO: 196) |
| 5′-ASO#12 | CAGGUAACAAACCAAC | U-ASO#08 | CGUUGACAGGACACGA |
| | (SEQ ID NO: 151) | | (SEQ ID NO: 197) |
| 5′-ASO#15 | ACCUUCCCAGGUAAC | U-ASO#09 | UAAUAACUAAUUACUGU |
| | (SEQ ID NO: 154) | | (SEQ ID NO: 198) |
| 5′-ASO#26 | AAACCAACCAACUUUC | | |
| | (SEQ ID NO: 150) | | |
| 5′-ASO#27 | CAGGUAACAAACCAAC | | |
| | (SEQ ID NO: 151) | | FSE region of ORF1a/b |
| 5′-ASO#29 | UUCCCAGGUAACAAAC | F-ASO#06 | UCGUAUACAGGGCUUU |
| | (SEQ ID NO: 153) | | (SEQ ID NO: 162) |
| | Spike coding region | F-ASO#23 | GGCUUUUGACAUCUAC |
| | | | (SEQ ID NO: 179) |
| S-ASO#11 | CAGUGUGUUAAUCUUAC | CRM0606 | CUGAUGUCGUAUACAG |
| | (SEQ ID NO: 97) | | (SEQ ID NO: 155) |
| S-ASO#14 | UGAGAUUCUUGACAUUA | CRM0607 | GCUUUUGACAUCUACA; |
| | (SEQ ID NO: 104) | | (SEQ ID NO: 156) |

the oligonucleotide hybridizes to a predetermined target site of human ACE2 mRNA region:

| | Target site | | Target site |
|---|---|---|---|
| ASO | sequence | ASO | sequence |
| CRM0443 | UAGGUUAUGAUGAGUGU | CRM0463 | CAAACCUUCCUAUUGAG |
| | (SEQ ID NO: 114) | | (SEQ ID NO: 127) |
| CRM0444 | ACCUAACCUUCUACUGU | CRM0464 | UUUGUAUUUGCUCACAG |
| | (SEQ ID NO: 115) | | (SEQ ID NO: 128) |
| CRM0446 | AAUGUGUCUUACGAGU | CRM0465 | CCCUUCUACUAUUUCUC |
| | (SEQ ID NO: 116) | | (SEQ ID NO: 129) |
| CRM0449 | CCCUUAUGUUCUUCCCU | CRM0466 | CCUAUCCUUCCUAUAUC |
| | (SEQ ID NO: 117) | | (SEQ ID NO: 130) |
| CRM0450 | CGGUAAACCUACACU | CRM0467 | GCUCCUAUACUACCGC |
| | (SEQ ID NO: 118) | | (SEQ ID NO: 131) |
| CRM0451 | GACAGUGAAUUGAUUAU | CRM0468 | UUAUAACUGUCUCCAAC |
| | (SEQ ID NO: 119) | | (SEQ ID NO: 132) |
| CRM0453 | UUCCAACCUCCUUCCAU | CRM0469 | ACGUGAACCUUCAAAC |
| | (SEQ ID NO: 120) | | (SEQ ID NO: 133) |
| CRM0454 | UUCCAACCUCCUUCCAU | CRM0470 | AACCUUCCUAUUGAGUA |
| | (SEQ ID NO: 120) | | (SEQ ID NO: 134) |
| CRM0456 | GUAGUAUGAUAAACAAU | CRM0472 | GUGUAAUAUGUAAGUGA |
| | (SEQ ID NO: 121) | | (SEQ ID NO: 135) |
| CRM0457 | CAUAUCUUAUCUGUCUG | CRM0473 | AACCAACUGUACUCGA |
| | (SEQ ID NO: 122) | | (SEQ ID NO: 136) |
| CRM0458 | GAGUUUGUGUAAUGUGG | CRM0475 | CGUGUUCCUUACUCCCA |
| | (SEQ ID NO: 123) | | (SEQ ID NO: 137) |
| CRM0459 | CGACCUGUUAUAAGG | CRM0476 | UACCUCCCUUCUCUACA |
| | (SEQ ID NO: 124) | | (SEQ ID NO: 138) |
| CRM0460 | ACCUUCUACUGUCUUCG | CRM0477 | CCGUGUGUGUUAUCAA; |
| | (SEQ ID NO: 125) | | (SEQ ID NO: 139) |
| CRM0462 | UAAUUUGCUAUCUACG | | |
| | (SEQ ID NO: 126) | | |

the oligonucleotide hybridizes to a predetermined target site of a SARS-CoV-2 viral RNA region and/or comprises a sequence of:

| | | |
|---|---|---|
| ASO#11/CRM0515 | GAAagttggttgGTTT (5′-3′) | 5′ leader target: AAACCAACCAACUUUC |
| | (SEQ ID NO: 54) | (SEQ ID NO: 150) |
| ASO#26/CRM0530 | GAaAGtTgGTtGgTTT(5′-3′) | 5′ leader target: AAACCAACCAACUUUC; |
| | (SEQ ID NO: 54) | (SEQ ID NO: 150) |

the oligonucleotide hybridizes to a predetermined target site of a SARS-CoV-2 viral RNA region and/or comprises a sequence of:

| | | FSE region of |
|---|---|---|
| ASO | Sequence | ORF1a/b target: |
| CRM0606 | CTgTAtACgACatCAG(5′-3′) | CUGAUGUCGUAUACAG |
| | (SEQ ID NO: 59) | (SEQ ID NO: 155) |
| CRM0607 | TGtAGaTGtCAaaAGC(5′-3′) | GCUUUUGACAUCUACA |
| | (SEQ ID NO: 60) | (SEQ ID NO: 156) |

comprising administering to the person a plurality of antisense oligonucleotides (ASOs), each hybridizing to a different target site of SARS-CoV-2 viral RNAs or human ACE2 mRNA and/or
comprising administering to the person a plurality of antisense oligonucleotides (ASOs), each hybridizing to a different target site of SARS-CoV-2 viral RNAs or human ACE2 mRNA, wherein a first of the antisense oligonucleotides (ASOs) hybridizes to predetermined target site of a SARS-CoV-2 viral RNA region and/or comprises a sequence of:

| | | |
|---|---|---|
| ASO#11/CRM0515 | GAAagttggttgGTTT (5′-3′) | 5′ leader target: AAACCAACCAACUUUC |
| | (SEQ ID NO: 54) | (SEQ ID NO: 150) or |
| ASO#26/CRM0530 | GAaAGtTgGTtGgTTT(5′-3′) | 5′ leader target: AAACCAACCAACUUUC |
| | (SEQ ID NO: 54) | (SEQ ID NO: 150); |

and a second of the antisense oligonucleotides (ASOs) hybridizes to a predetermined target site of a SARS-CoV-2 viral RNA region and/or comprises a sequence of:

| | | FSE region of |
|---|---|---|
| ASO | Sequence | ORF1a/b target: |
| CRM0606 | CTgTAtACgACatCAG(5′-3′) | CUGAUGUCGUAUACAG |
| | (SEQ ID NO: 59) | (SEQ ID NO: 155) |
| CRM0607 | TGtAGaTGtCAaaAGC(5′-3′) | GCUUUUGACAUCUACA |
| | (SEQ ID NO: 60) | (SEQ ID NO: 156) |

In an aspect the invention provides a pharmaceutical composition configured for a method of treating COVID-19 herein, and comprising a locked nucleic acid antisense oligonucleotide (LNA ASO) targeting SARS-CoV-2 viral RNAs or human ACE2, and a pharmaceutically acceptable excipient, in bulk multidosage, or unit dosage.

In an aspect the invention provides a pharmaceutical composition configured for a method of treating COVID-19 herein, and comprising a plurality of locked nucleic acid antisense oligonucleotides (LNA ASOs) each targeting a different site of SARS-CoV-2 viral RNAs or human ACE2, and a pharmaceutically acceptable excipient, in bulk multidosage, or unit dosage.

In an aspect the invention provides a pharmaceutical delivery device, such as syringe, inhaler or nebulizer comprising a pharmaceutical composition configured for the method of treating COVID-19 according to claim 1, and comprising a locked nucleic acid antisense oligonucleotide (LNA ASO) targeting SARS-CoV-2 viral RNAs or human ACE2, and a pharmaceutically acceptable excipient, in bulk multidosage, or unit dosage.

In an aspect the invention provides a pharmaceutical delivery device, such as syringe, inhaler or nebulizer comprising a pharmaceutical composition configured for the method of treating COVID-19 according to claim 1, and comprising a plurality of locked nucleic acid antisense oligonucleotides (LNA ASOs) each targeting a different site of SARS-CoV-2 viral RNAs or human ACE2, and a pharmaceutically acceptable excipient, in bulk multidosage, or unit dosage.

The invention encompasses all combinations of the particular embodiments recited herein, as if each combination had been laboriously recited.

### BRIEF DESCRIPTION OF THE DRAWINGS

FIGS. 1a-e. In vitro screening of LNA ASOs targeting SARS-CoV-2. a) Schematic representation of the genome structure and the regions targeted by LNA ASOs. b) and c) The SARS-CoV-2 WA1 strain-infected Huh-7 cells treated with LNA ASO and cell culture medium. d) Dose-dependent effects of 5′-ASO #26 were evaluated in infected Huh-7 cells with increasing doses of the LNA ASO by RT-qPCR. e) The infectious virus was measured by TCID₅₀ assay.

FIGS. 2a-d. 5′-ASO #26 disrupts the stem-loop structure of SL1. a) Scatter plots comparing 5′-leader structures in vitro in different conditions. b) Scatter plots comparing 5′-leader structures in infected Huh-7 cells at different time points. C-d) Structural model of SL1 predicted from the DMS-MaPseq of in vitro-transcribed 5′leader RNA with the addition of control LNA ASO (c) and 5′-ASO #26 (d).

FIGS. 3a-f. Once-daily treatment of 5′-ASO #26 represses the generation of infectious virus in mice. a) Treatment course of once-daily 5′-ASO #26 administration. b) Viral burden in lungs of mice treated with Salin or LNA ASO measured by TCID₅₀ assay. c) Levels of viral RNAs encoding Spike and nucleocapsid protein measured in mouse lungs by RT-qPCR. d) IHC staining of SARS-CoV-2 Protein N in all infected K18-hACE2 mice with or without LNA ASO treatment. e) Expression changes of SARS-CoV-2 infection-upregulated genes in Saline- and LNA ASO-treated groups. f) Gene set enrichment analysis of Hallmark gene sets enriched in lungs of Saline- or LNA ASO-treated mice.

FIGS. 4a-f. Prophylaxis and treatment of SARS-CoV-2 strains with 5′-ASO #26. a) Mice administered with different treatment regimens of 5′-ASO #26. b) Dose-dependent effects of 5′-ASO #26 on inhibition of viral replication of SARS-CoV-2 WA1 and B.1.351 strains in infected Huh-7 cells by RT-qPCR. c) and e) Viral burden in lungs of mice treated with Saline and 5′-ASO #26 measured by TCID₅₀ assay using lung homogenates. d) and f) Weight change monitored.

FIGS. 5a-b. In vitro screening of LNA ASOs targeting SARS-CoV-2. a) and b) Infected Huh-7 cells were treated with LNA ASO and cell culture media was collected at 48 hpi.

FIGS. 6a-e. Physiological and histological analysis of LNA ASO-treated K18-hACE2 mice. a) Weight change of mice monitored over the treatment/infection course. b) LNA ASO dose-dependence efficacy testing in mice. c) Representative images of hematoxylin and eosin staining of lung sections and immunohistochemistry staining of CD3 and B220 in infected K18-hACE2 mice with Saline or 5′-ASO #26 treatment. d) Viral burden in lungs of hamsters treated with Saline or 5′-ASO #26. e) Weight change of hamsters monitored over the treatment/infection course.

FIGS. 7a-e. Evaluating the in vivo effects of 5′-ASO #26 in K18-hACE2 mice. a) Percentage of human/viral sequencing reads in RNA-seq data of Saline- and LNA ASO-treated mouse lung. b) Expression change of SARS-CoV-2 infection-downregulated genes in Saline- and LNA ASO-treated group. c) GSEA of REACTOME gene sets enriched among upregulated genes in lungs of Saline-treated mice. d) GSEA plot and heatmap of significantly upregulated genes enriched in cholesterol homeostasis pathway in ASO-treated mice. e) Heatmaps of significantly upregulated and downregulated genes at day 4 of SARS-CoV-2 infection.

FIGS. 8a-c. 5′-ASO #26 is capable of repressing the replication of SARS-CoV-2 variant strains. a) Representative images of IHC staining of SARS-CoV-2 nucleocapsid protein in Saline or Prophylactic #2 regimen groups. b) Dose-dependent efficacy of 5′-ASO #26 in repressing replication of SARS-CoV-2 variant strains. c) Viral RNA levels in mouse lungs.

FIGS. 9a-d. a) Normalized DMS signal in vitro v. normalized DMS signal in vitro control; b) SARS-CoV-2 FSE structural model; c) Assay design schematic; d) Frameshifting rate.

FIGS. 10a-c. Human ACE2 ASO-1 toxicity luminescence assay at 5, 10, 50, 100 nM: a) CRM443, CRM444, CRM446, CRM449, CRM450, CRM451, CRM453, CRM454, CRM456; b) CRM457, CRM458, CRM459, CRM460, CRM462, CRM463, CRM464, CRM465, CRM466; c) CRM467, CRM468, CRM469, CRM470, CRM472, CRM473, CRM475, CRM476, CRM477.

FIG. 11. SARS-Cov-2 Spike expression inhibition by 5′ leader sequence LNA ASOs.

FIG. 12. SARS-Cov-2 Protein N (set 1) inhibition by 5′ leader sequence LNA ASOs.

FIG. 13. SARS-Cov-2 Spike expression inhibition by ACE2 LNA ASOs.

FIG. 14. SARS-Cov-2 Protein N (set 1) expression inhibition by ACE2 LNA ASOs.

FIG. 15. SARS-Cov-2 Protein N (set 1) expression inhibition by panel of LNA ASOs.

FIG. 16. SARS-Cov-2 Spike expression inhibition by panel of LNA ASOs.

FIG. 17. SARS-Cov-2 Spike expression inhibition by panel of LNA ASOs.

FIG. 18. SARS-Cov-2 Protein N expression inhibition by panel of LNA ASOs.

FIG. 19. SARS-Cov-2 Protein N expression inhibition by CRM606/607.

FIG. 20. SARS-Cov-2 Spike expression inhibition by CRM606/607.

FIG. 21. SARS-Cov-2 Protein N expression inhibition by frameshifting LNA ASOs.

FIG. 22. SARS-Cov-2 Spike expression inhibition by frameshifting LNA ASOs.

FIG. 23. SARS-Cov-2 Protein N expression inhibition by frameshifting LNA ASOs.

FIG. 24. SARS-Cov-2 Spike expression inhibition by frameshifting LNA ASOs.

FIG. 25. SARS-Cov-2 Spike expression inhibition by CRM607; dose responses in different variant strains.

### DESCRIPTION OF PARTICULAR EMBODIMENTS OF THE INVENTION

Unless contraindicated or noted otherwise, in these descriptions and throughout this specification, the terms “a” and “an” mean one or more, the term “or” means and/or. The examples and embodiments described herein are for illustrative purposes only and various modifications or changes in light thereof will be suggested to persons skilled in the art and are to be included within the spirit and purview of this application and scope of the appended claims. All publications, patents, and patent applications cited herein, including citations therein, are hereby incorporated by reference in their entirety for all purposes.

Large-scale manufacture of therapeutic LNA ASOs is well established, and LNA ASOs are very stable and therefore do not require refrigeration like biologic drugs, such as antibodies and vaccines. Candidate compounds are readily tested, for example, by intranasal delivery or by inhalation in dose-escalation studies, or by subcutaneous injection in a humanized transgenic mouse model and Syrian hamsters, and testing in non-human primates (Rhesus macaques). LNA ASO can be verified with inhalation studies in non-human primates, and GMP production can be readily scaled up for human testing with delivery through an established inhaler or nebulizer.

### Example: Therapeutic Targeting of SARS-CoV-2 Using LNA-Modified Antisense Oligonucleotides for the Treatment of COVID-19 Patients with Acute Respiratory Disease

Abstract

The COVID-19 pandemic is exacting an increasing toll worldwide, with new SARS-CoV-2 variants emerging that exhibit higher infectivity rates and that may partially evade vaccine and antibody immunity¹. Rapid deployment of non-invasive therapeutic avenues capable of preventing infection by all SARS-CoV-2 variants could complement current vaccination efforts and help turn the tide on the COVID-19 pandemic². Here, we describe a novel therapeutic strategy targeting the SARS-CoV-2 RNA using locked nucleic acid antisense oligonucleotides (LNA ASOs). We identified an LNA ASO binding to the 5′ leader sequence of SARS-CoV-2 ORF1a/b that disrupts a highly conserved stem-loop structure with nanomolar efficacy in preventing viral replication in human cells. Daily intranasal administration of this LNA ASO in the K18-hACE2 humanized COVID-19 mouse model potently (98-99%) suppressed viral replication in the lungs of infected mice, revealing strong prophylactic and treatment effects. We found that the LNA ASO also represses viral infection in golden Syrian hamsters, and is highly efficacious in countering all SARS-CoV-2 “variants of concern” tested in vitro and in vivo, including B.1.427, B.1.1.7, and B.1.351 variants³. Hence, inhaled LNA ASOs targeting SARS-CoV-2 represents a promising therapeutic approach to reduce transmission of variants partially resistant to vaccines and monoclonal antibodies, and could be deployed intranasally for prophylaxis or via lung delivery by inhaler or nebulizer to decrease severity of COVID-19 in infected individuals. LNA ASOs are chemically stable and can be flexibly modified to target different viral RNA sequences⁴, and they may have particular impact in areas where vaccine distribution is a challenge, and could be stockpiled for future coronavirus pandemics.

Main

To address the challenges described above, we developed a novel strategy to inhibit the replication of SARS-CoV-2 using antisense oligonucleotides (ASOs) targeting viral RNAs. ASOs, which rely on Watson-Crick base-pairing to target specific complementary RNA sequences, can be quickly designed to target any viral or host RNA sequence, including non-coding structural elements that may be important for viral replication, and may recruit RNase H for cleavage (gapmers) or act through steric hindrance (mixmers)⁴. ASOs are typically well tolerated, and a number of ASO therapeutics have been approved for clinical use¹². Additionally, ASO manufacturing is well established and can be readily scaled-up. We have employed chemically modified gapmer and mixmer ASOs containing interspersed locked nucleic acid nucleotide bases (LNAs) and DNA nucleotides linked by phosphorothioate (PS) bonds. The introduced chemical modifications confer increased affinity, stability and improved pharmacokinetic/pharmacodynamic properties^{13,14,15}.

SARS-CoV-2 is a compact (30 kilobases) positive-sense single-stranded RNA virus, with a 5′ untranslated region (UTR), the ORF1a/b RNA encoding non-structural viral proteins, and a 3′ segment encoding the structural RNAs, such as the Spike protein that binds to the ACE2 receptor on host cells, and the nucleocapsid N protein involved in virion assembly, and a 3′UTR¹⁶. The 5′ UTR, a non-coding segment consisting of multiple highly conserved stem-loop and more complex secondary structures, is functionally critical for viral translation and replication by affording protection from host cell antiviral defenses and through selective promotion of viral transcript translation over those of the host cell, at least in part through the recruitment of the viral Nsp1 protein¹⁷. The 5′ UTR begins with a short 5′ leader sequence (nucleotides 1-69), which is added via discontinuous transcription to the 5′ end of all sub-genomic RNA transcripts encoding the viral structural proteins, and regulates their translation as well as translation of ORF1a/b from full-length genomic RNA¹⁸. The ORF1a/b also contains a structured and highly conserved frameshift stimulation element (FSE) near its center that controls a shift in the protein translation reading frame by one nucleotide of ORF1a/b genes 3′ to the FSE. The FSE and accurate frame shifting is crucial for the expression of ORF1b, which encoded proteins such as the RNA-dependent RNA polymerase (RdRP) involved in SARS-CoV-2 genome replication¹⁹.

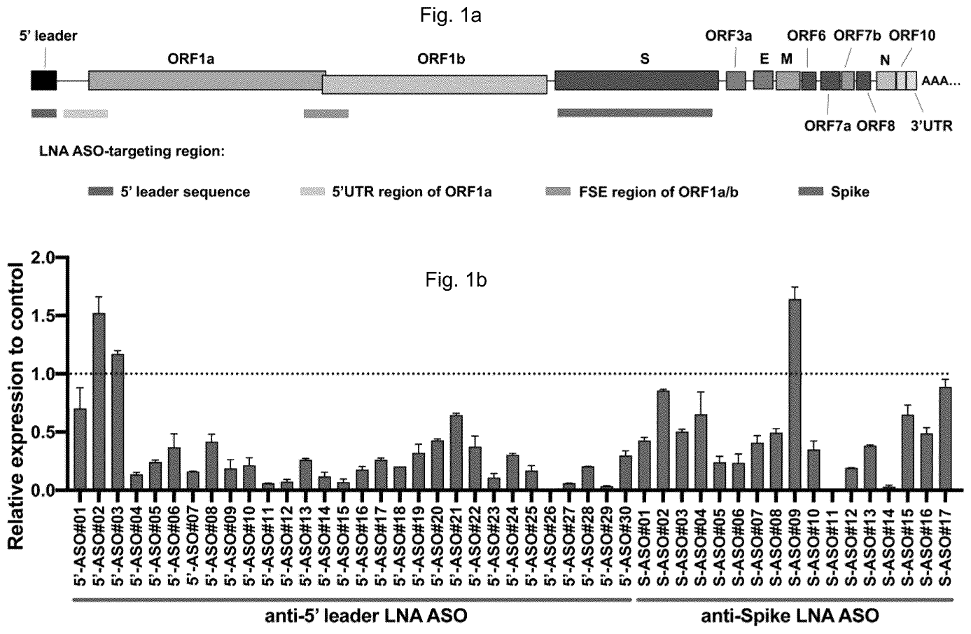

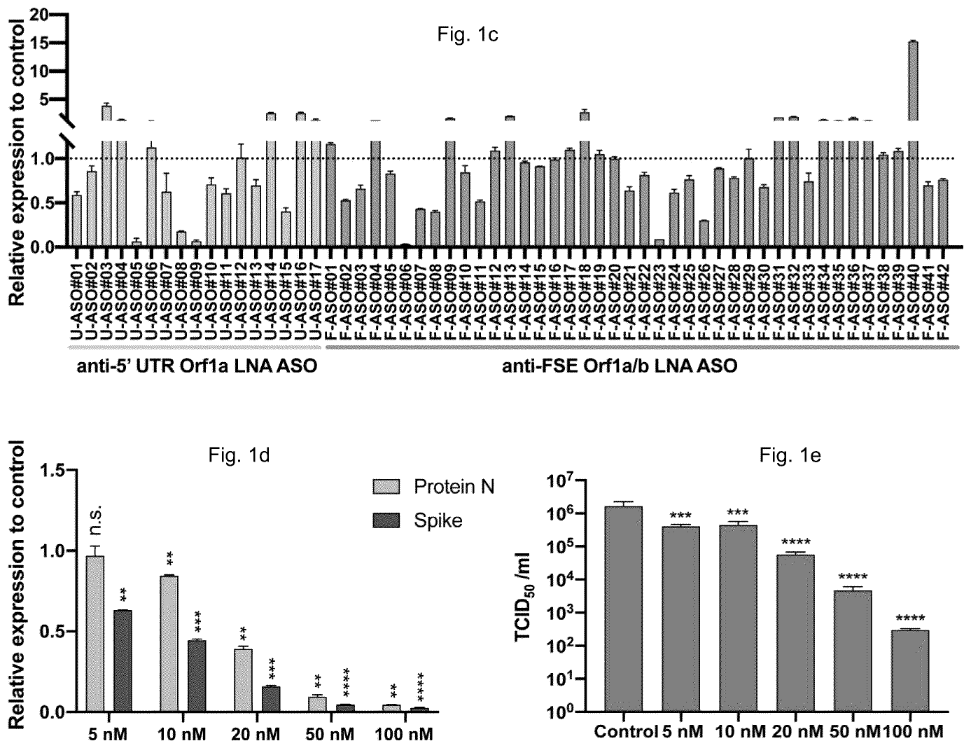

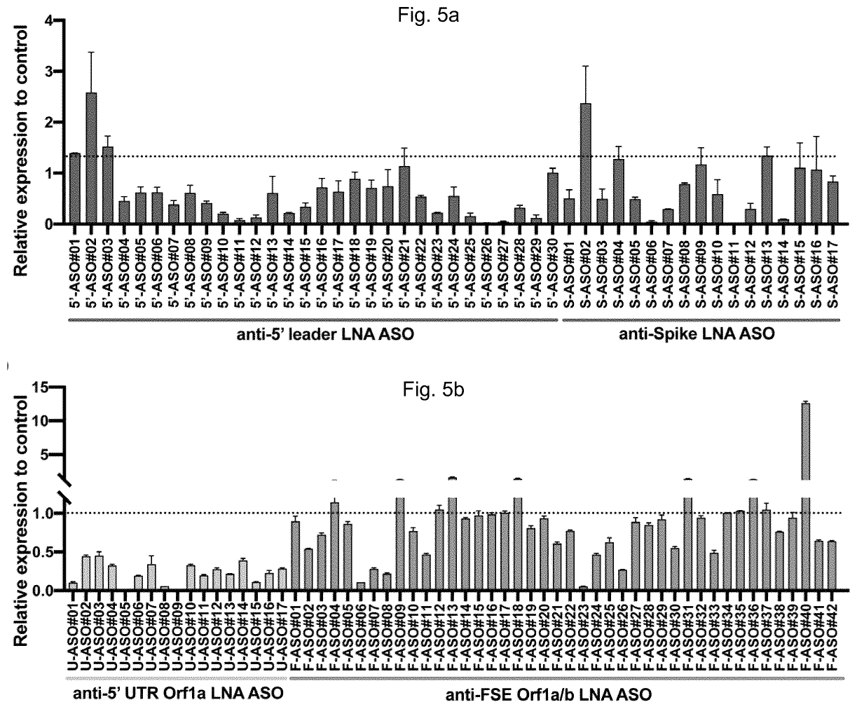

We designed multiple LNA ASOs targeting the 5′ leader sequence, downstream sequences in the 5′ untranslated region (UTR) of ORF1a/b, and the ORF1a/b FSE of SARS-CoV-2 (FIG. 1a and Extended Data Table 1). Additionally, given the importance of Spike protein in host cell entry for SARS-CoV-2 infection, LNA ASOs targeting the Spike coding sequence were also tested (FIG. 1a and Extended Data Table 1). To evaluate the viral repressive effect of LNA ASOs, the initial screening was carried out in Huh-7 human hepatoma cells, which exhibit excellent transfection efficiency and are readily infected by SARS-CoV-2. Cells transfected with LNA ASOs were infected with SARS-CoV-2 and both cells and medium were collected at 48 h post-infection (hpi) for RNA extraction and infectious viral particle determination, respectively. The viral titer was measured by detecting the expression level/copy number of nucleocapsid protein (Protein N) and Spike using reverse transcription (RT)-quantitative PCR (qPCR). The screening results showed that the treatment with certain LNA ASOs lead to a dramatic decrease of Protein N and Spike expression (FIGS. 1b and 1c, FIGS. 5a and 5b).

We found that LNA ASOs targeting the 5′ leader region of SARS-CoV-2 were particularly effective in suppressing viral RNA levels in infected cells (FIG. 1b and FIG. 5a). This is consistent with the fact that the 5′ leader sequence is present in all viral RNA transcripts and is required for viral replication. The most potent LNA ASO targeting the 5′ leader, 5′-ASO #26, was selected for further investigation of its viral repression capability. The repressive effect of 5′-ASO #26 was demonstrated in a dose-dependent manner by measuring the expression level of viral RNAs (FIG. 1d) and by directly determining the viral titer with the Median Tissue Culture Infectious Dose (TCID₅₀) assay in Vero E6 African green monkey kidney epithelial cells (FIG. 1e).

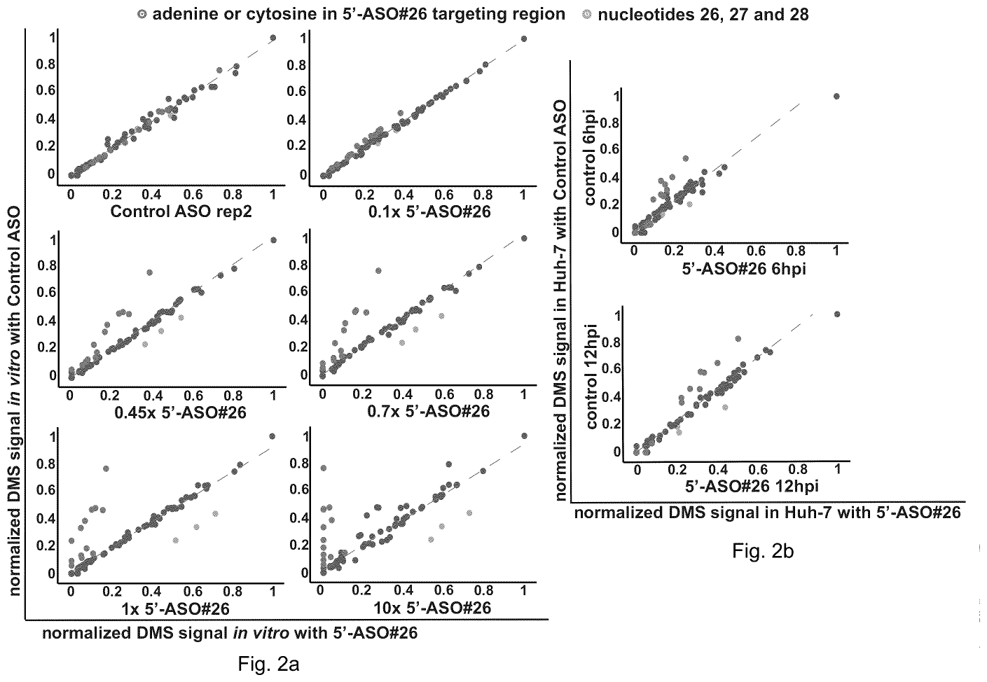

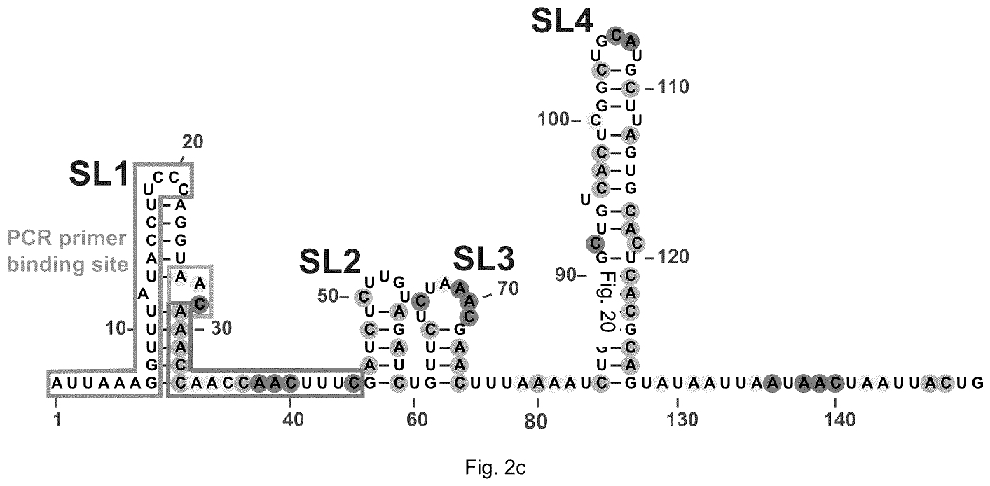

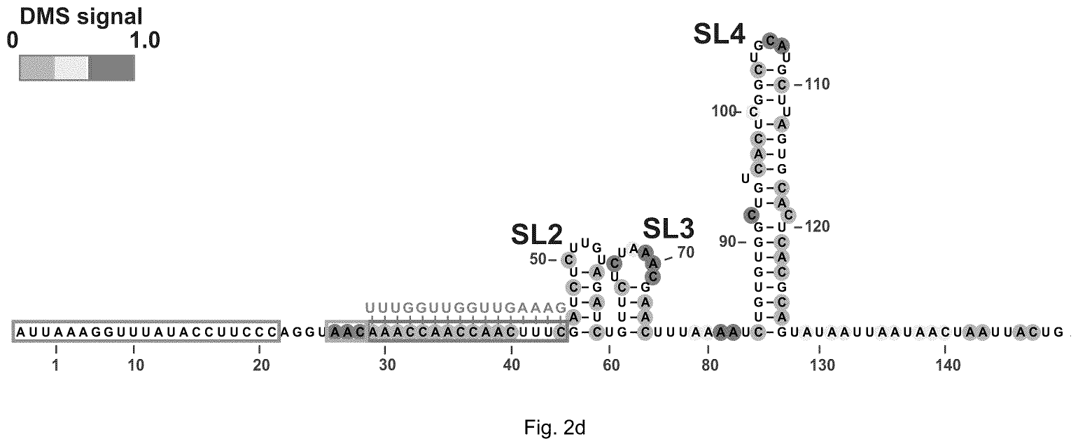

Although the 5′ UTR nucleotide sequences are somewhat divergent amongst the coronavirus family, the secondary structure of the 5′ UTR is highly conserved²⁰, and it has been shown that two stem-loop structures, SL1 and SL2, are formed by the 5′ leader sequence²¹. Since the complementary sequence of 5′-ASO #26 aligns along the 3′ portion of SL1 (marked in pink frame) (FIG. 2c), we hypothesized that the viral repressive effect of 5′-ASO #26 may be in part due to its ability to disrupt the secondary structure of the 5′ leader sequence upon binding to the viral genomic or sub-genomic RNAs, interfering with the formation of the SL1 stem-loop structure. To test if 5′-ASO #26 can disrupt the SL1 structure, we carried out in vitro transcription of the SARS-CoV-2 5′ UTR sequence, added 5′-ASO #26 and treated samples with dimethyl sulfate (DMS). DMS is able to specifically and rapidly methylate unpaired adenines (A) and cytosines (C) within single-stranded sequences and not those that are complexed as RNA secondary structure or to the LNA ASO, allowing for the unpaired A or C nucleotides to be detected by DMS-MaPseq²². Our results strongly indicated that the secondary structure of SL1 was indeed interrupted by 5′-ASO #26 in a dose-dependent manner (FIG. 2a). We also performed DMS-MaPseq with SARS-CoV-2-infected Huh-7 cells in the presence or absence of 5′-ASO #26, and monitored secondary structure changes of SL1 at 6 hpi and 12 hpi. Similar to the findings with the in vitro assay, the result from infected cells confirmed the disruption of the secondary structure of SL1 due to binding of 5′-ASO #26 (FIG. 2b).

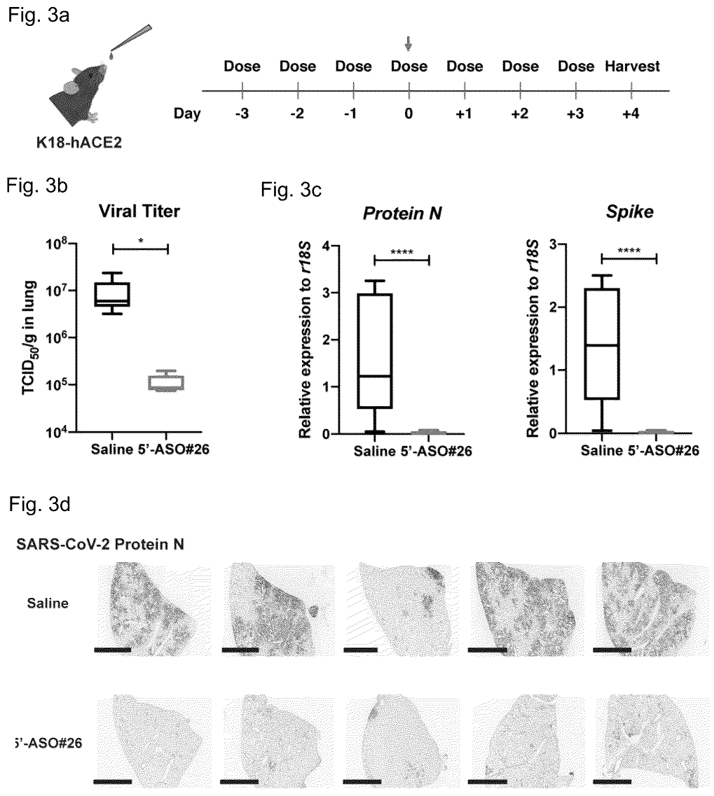

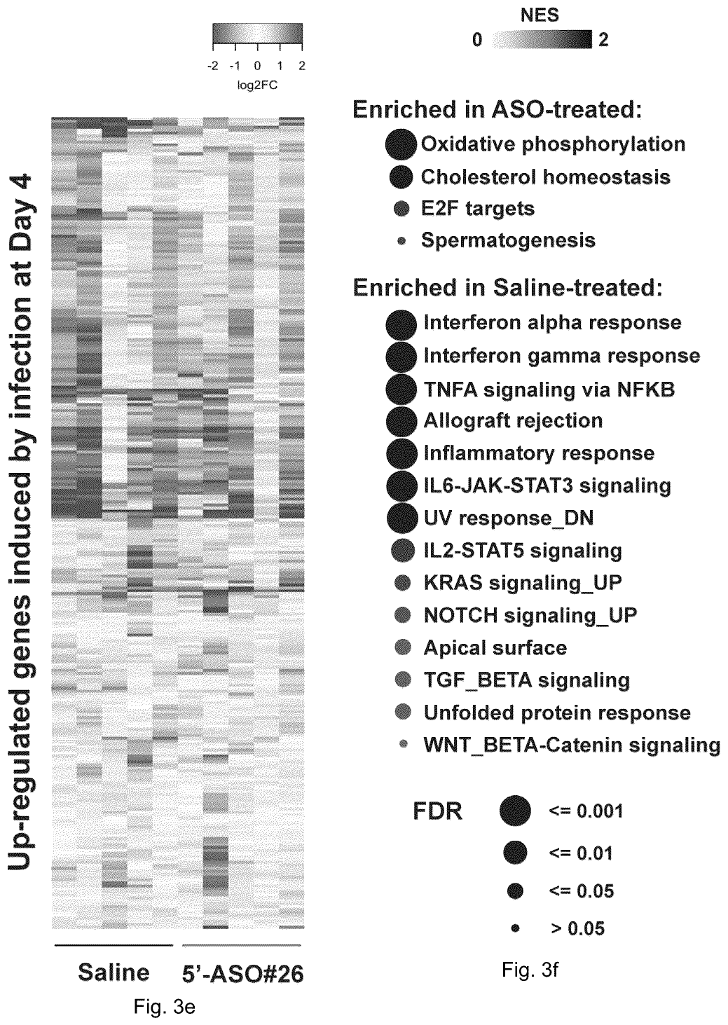

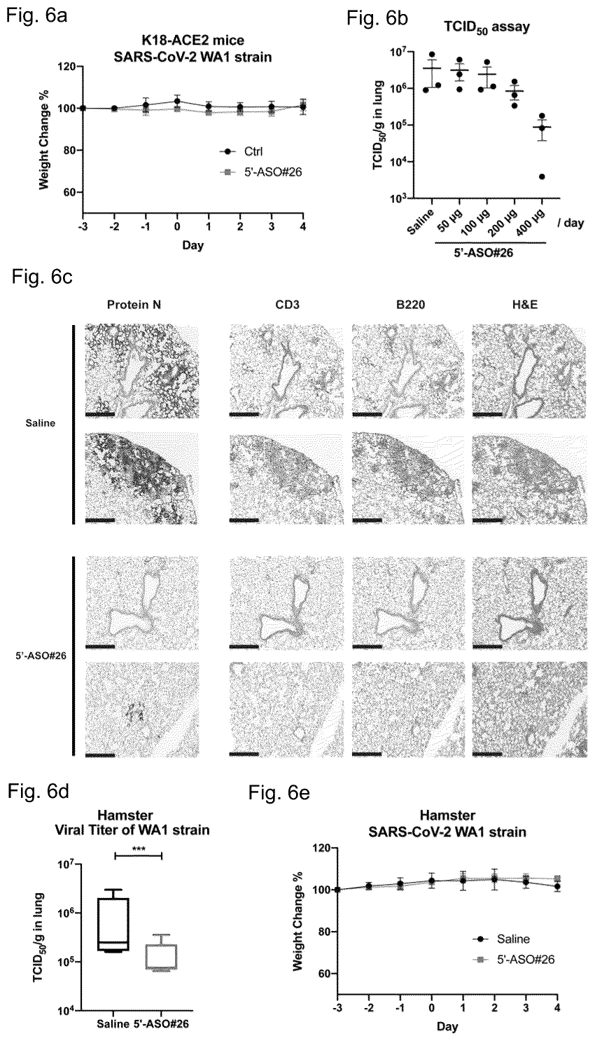

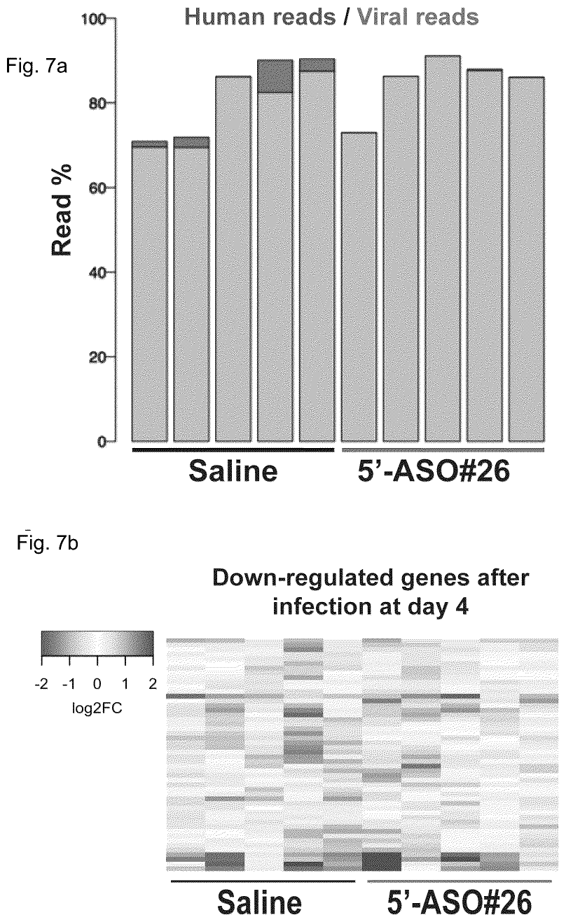

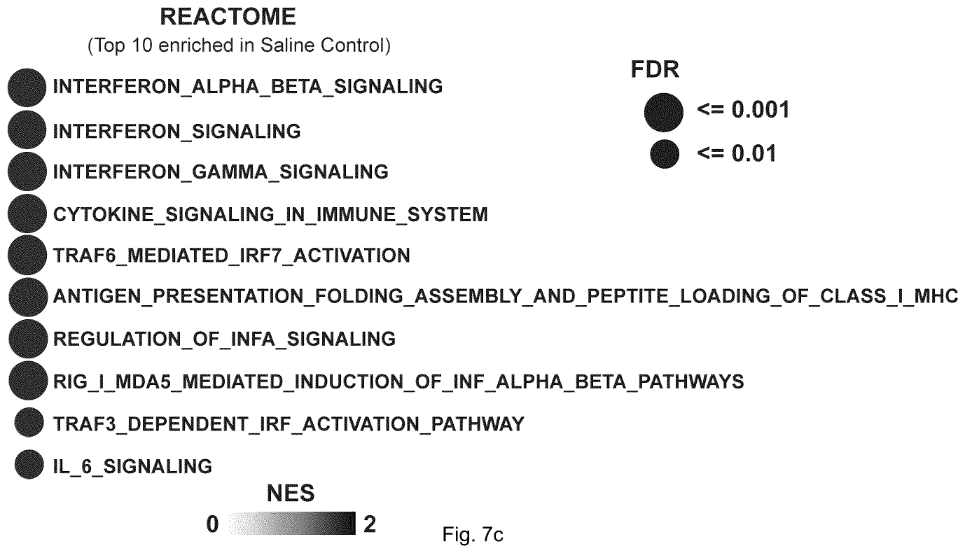

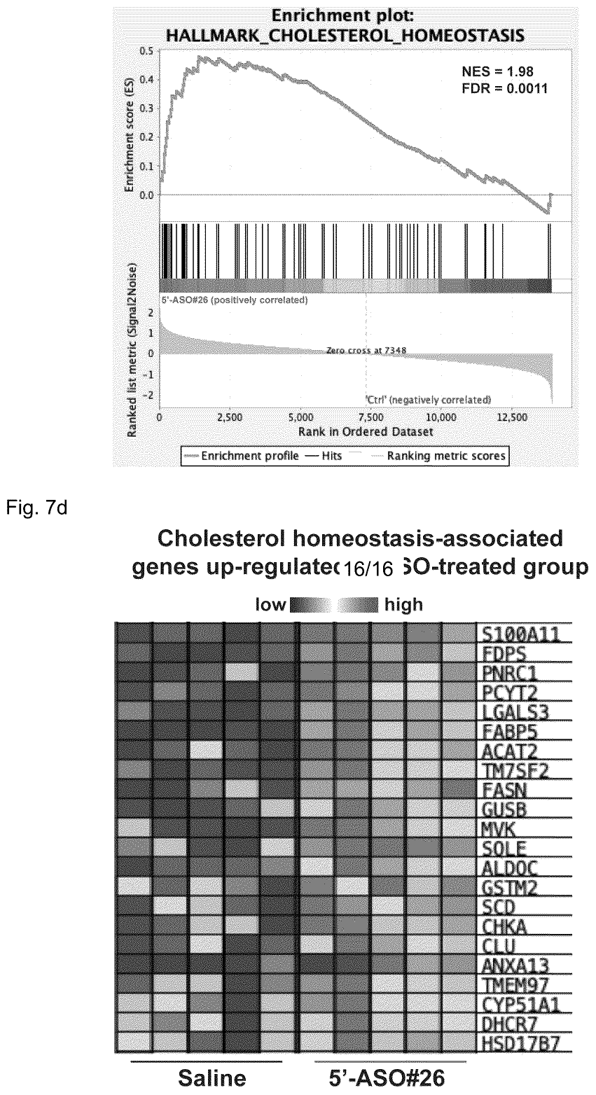

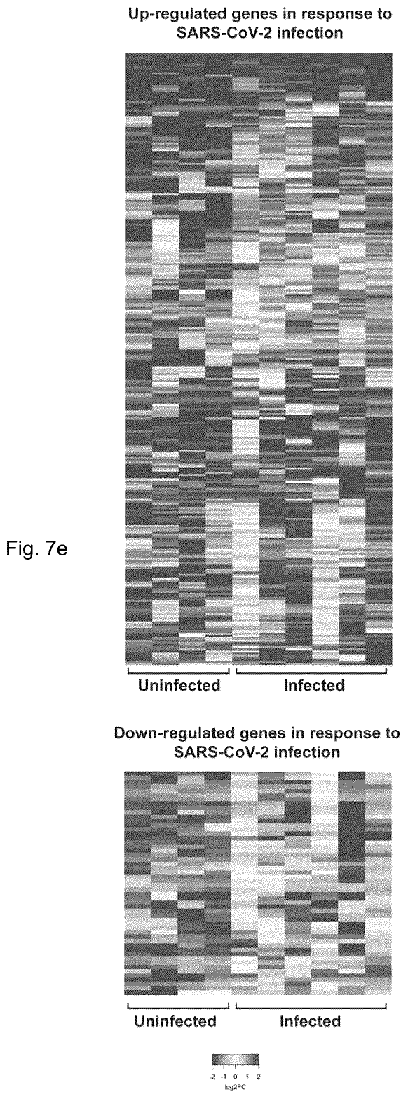

To evaluate the effects of 5′-ASO #26 in vivo, we employed humanized transgenic K18-hACE2 mice, which are expressing human ACE2 allowing SARS-CoV-2 cell entry and infectious spread²³. K18-hACE2 mice were inoculated with 1×10⁴ TCID⁵⁰ USA-WA1/2020 strain via intranasal administration. No significant weight loss was observed at 4 days post-infection (dpi) (FIG. 6a), consistent with previous studies indicating that weight loss starts from 5 dpi²³. Mice were treated with 5′-ASO #26 (400 μg) once-daily via intranasal administration in saline, from 3 days before infection until 3 dpi (FIG. 3a). High levels of infectious SARS-CoV-2 viral particles were detected in the lungs of the saline-treated control group (Saline), whereas a remarkable decrease (>80-fold) in the viral load in lungs was observed in the LNA ASO-treated group (FIG. 3b). As expected, the levels of viral Protein N and Spike RNA were also potently (98-99%) repressed after LNA ASO treatment, as measured by RT-qPCR (FIG. 3c). Previous studies have shown that golden Syrian hamsters can be infected with SARS-CoV-2²⁴. We inoculated hamsters with 10 TCID₅₀ USA-WA1/2020 strain and treated them with 5′-ASO #26 (600 μg) following the same schedule as for the mouse studies (FIG. 3a). About 10-fold decreased viral load in lung was observed in the LNA ASO-treated group and with no significant weight change (FIG. 6d, 6e). Considering that the dose was approximately 2.5-fold lower per kg, the viral replication was still efficiently repressed in hamsters. To further investigate the physiological effects of LNA ASO in mice, histological analyses were carried out with control and LNA ASO-treated lung tissue after SARS-CoV-2 infection. The analyses revealed clear repression of Protein N expression with LNA ASO treatment (FIG. 3d). Meanwhile, we did not observe significant histological changes by H&E staining in either Saline or LNA ASO-treated mice, which is consistent with previous studies demonstrating that significant inflammation and severe alveolar wall thickening could only be observed at 7 dpi²³. However, we did notice that the localized signal of Protein N correlated with local enrichment of CD3 (T cell marker), B220 (B cell marker) by immunohistochemistry, and moderate thickening of the alveolar wall (by H&E staining) in Saline-treated mice, but not in LNA ASO-treated mice (FIG. 6c). The viral repressive effect of the LNA ASO was also examined by RNA-seq of lung tissues from mice with saline or LNA ASO treatment. Consistent with results from the TCID₅₀ assay, viral reads were dramatically decreased in LNA ASO-treated mice when compared with that of the Saline control (FIG. 7a). To evaluate the in vivo effect of 5′-ASO #26, previous RNA-seq data from infected K18-ACE2 mice²³ was used to define up- and down-regulated genes in response to SARS-CoV-2 infection at 4 dpi (FIG. 7e). As expected, expression profile changes induced by infection were markedly rescued by LNA ASO treatment (FIG. 3e and FIG. 7b). Gene set enrichment analysis (GSEA) revealed strong enrichment of gene involved in type I and II IFN signaling in infected mice treated with Saline (FIG. 3f and FIG. 7c), which is consistent with previous studies showing induction of anti-viral defenses²³. Interestingly, the cholesterol homeostasis gene pathway was enriched in LNA ASO-treated mice (FIG. 3f and FIG. 7d). It has been reported that the cholesterol biosynthesis pathway is necessary for SARS-CoV-2 infection^{25,26} and decreased levels of total cholesterol, high-density lipoprotein (HDL), and low-density lipoprotein (LDL) were reported in COVID-19 patients²⁷, indicating a comprehensive role of intracellular and extracellular cholesterol during SARS-CoV-2 infection. The restored cholesterol homeostasis in the lungs of LNA ASO-treated mice further indicates the efficacy of the LNA ASO treatment.

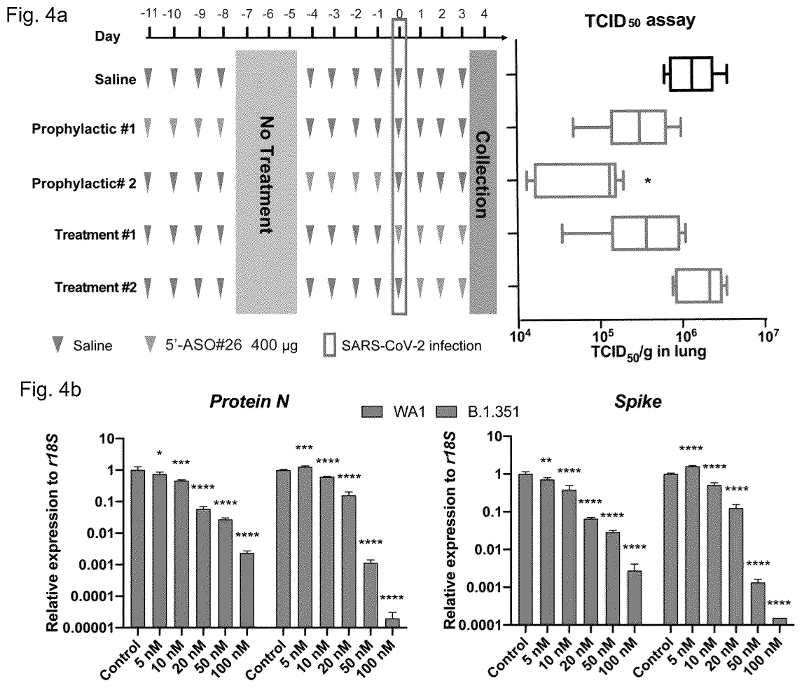

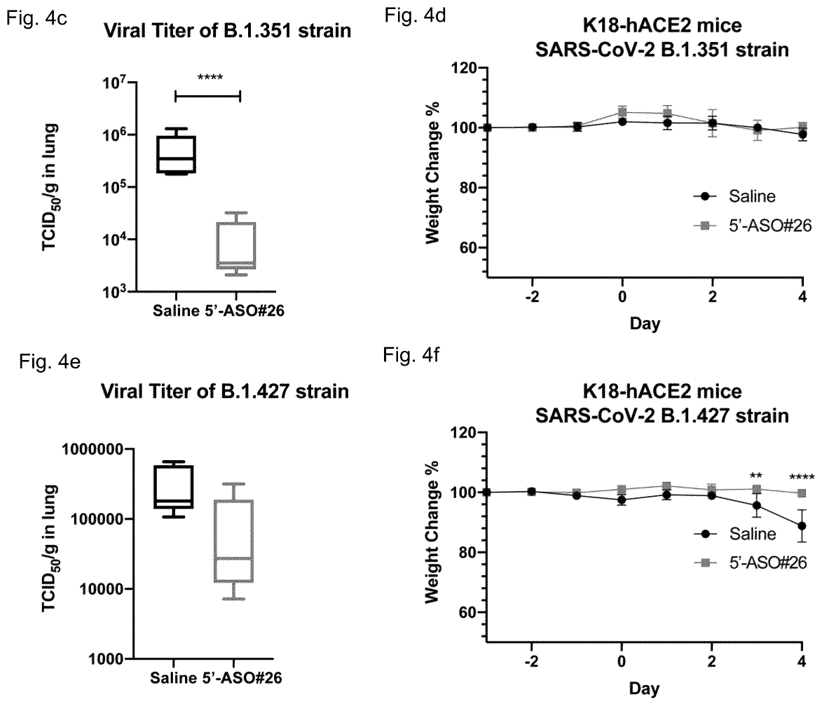

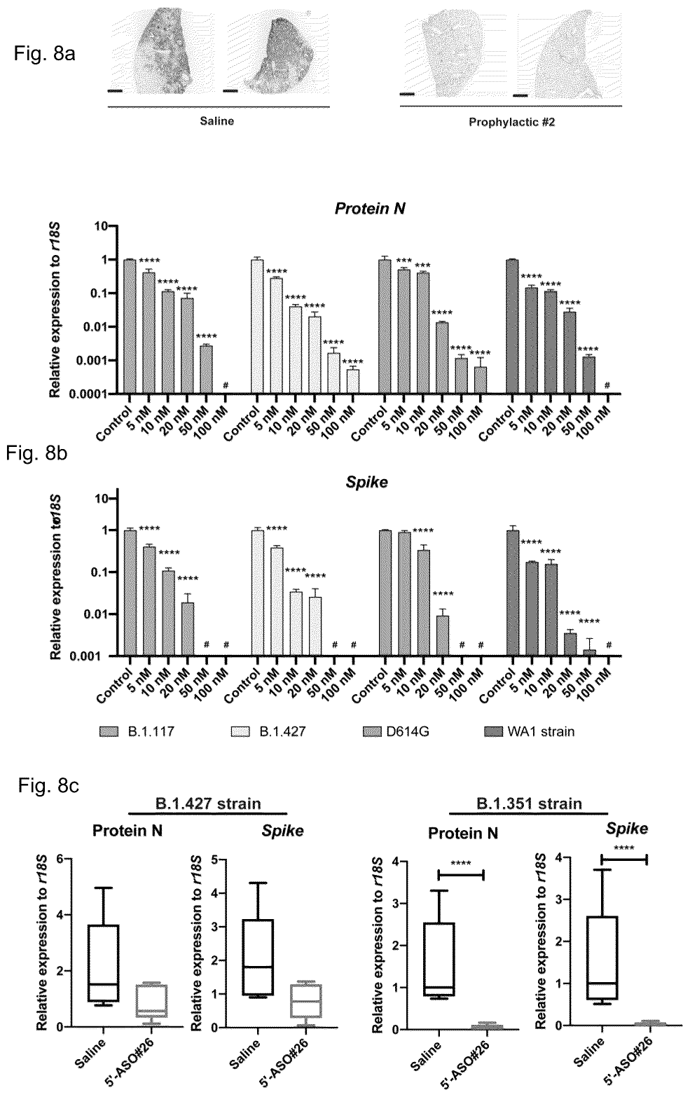

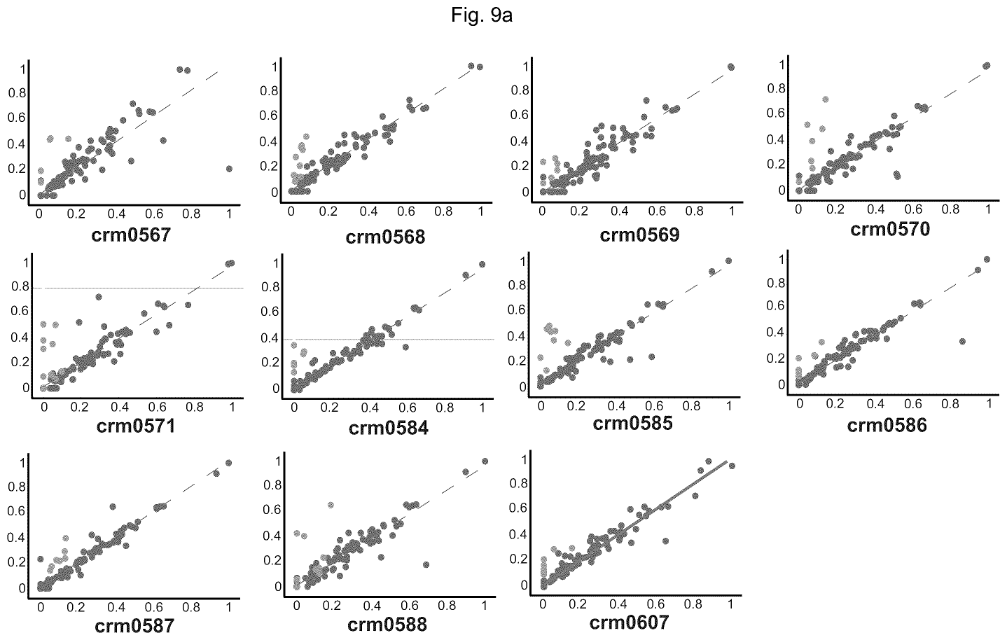

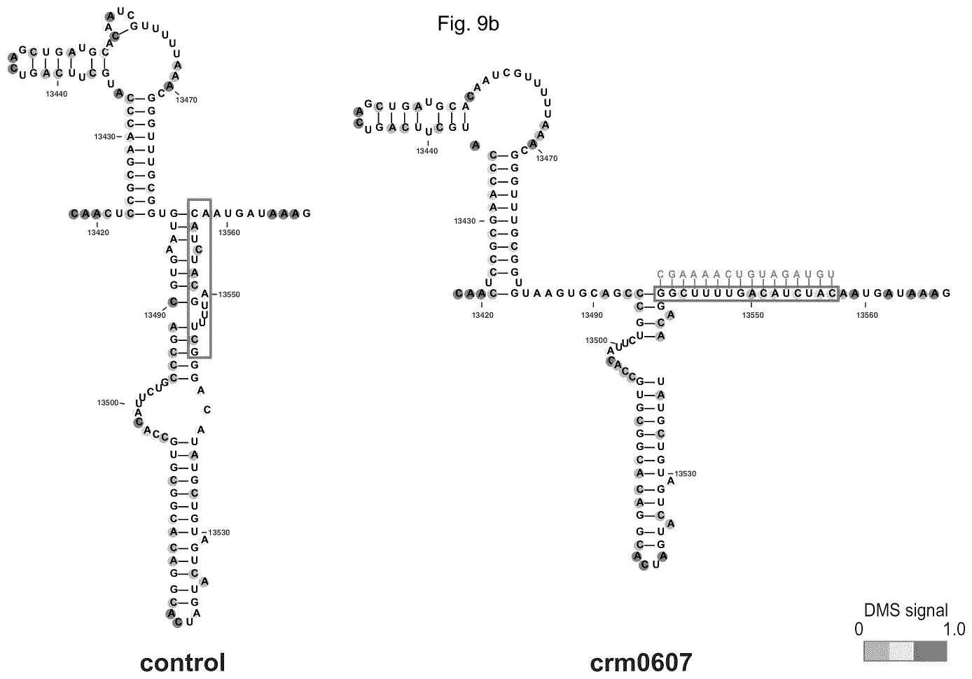

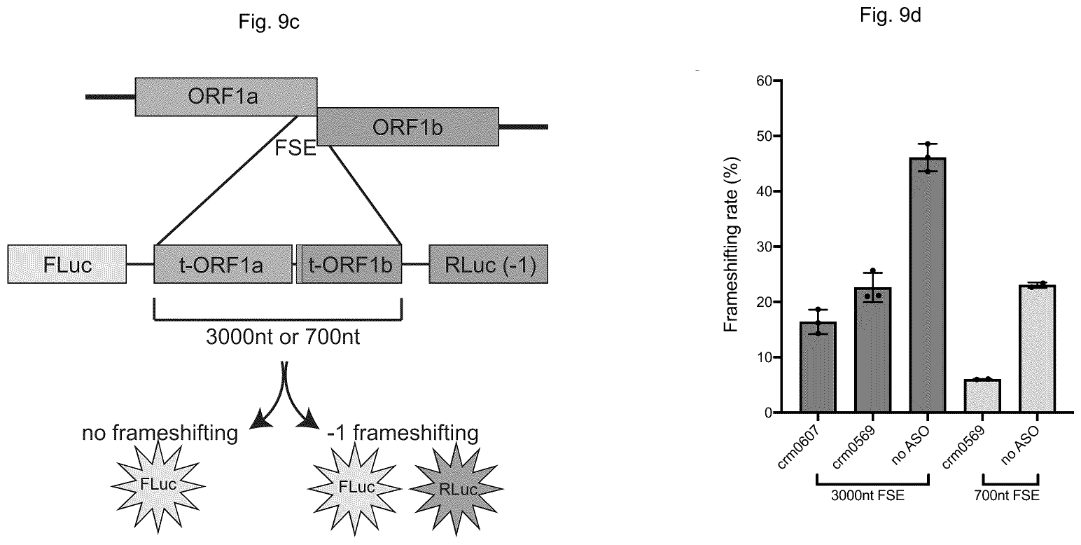

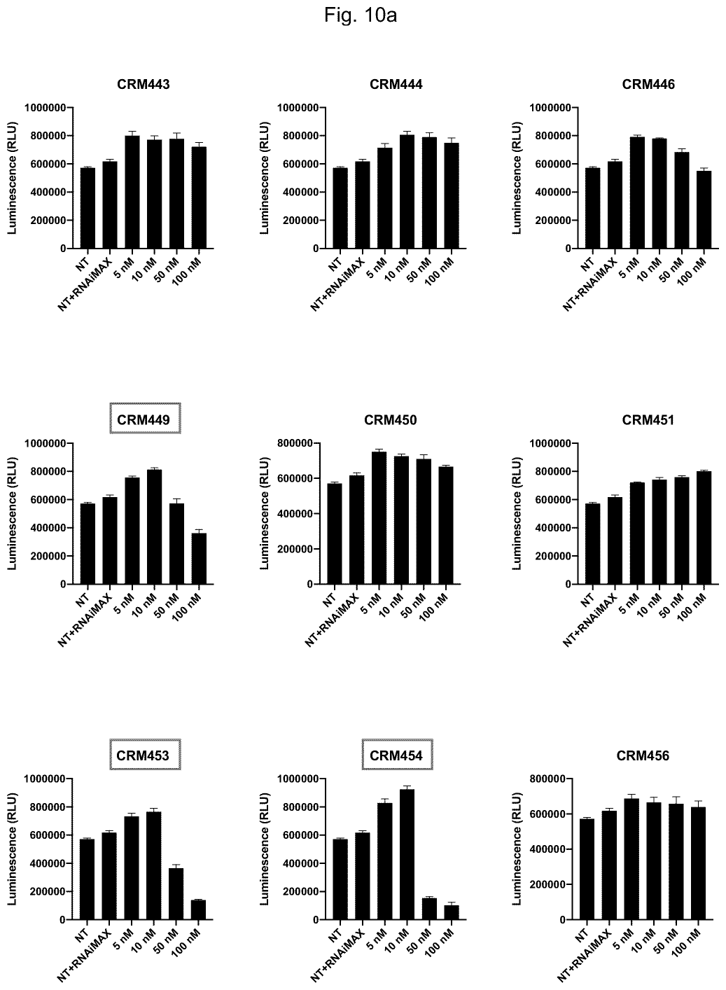

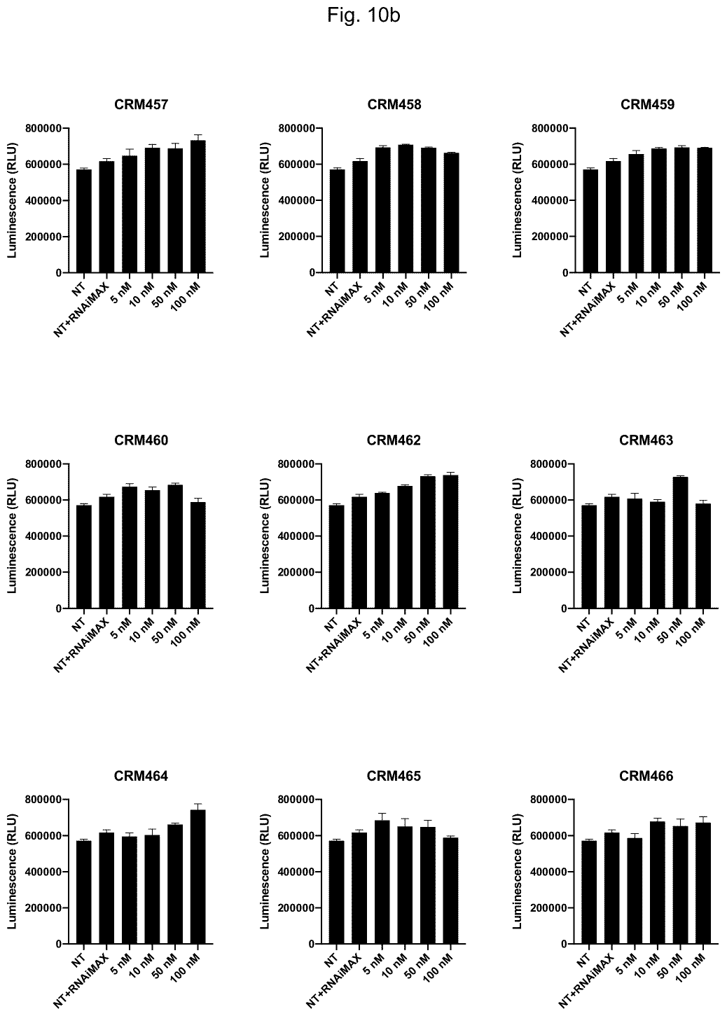

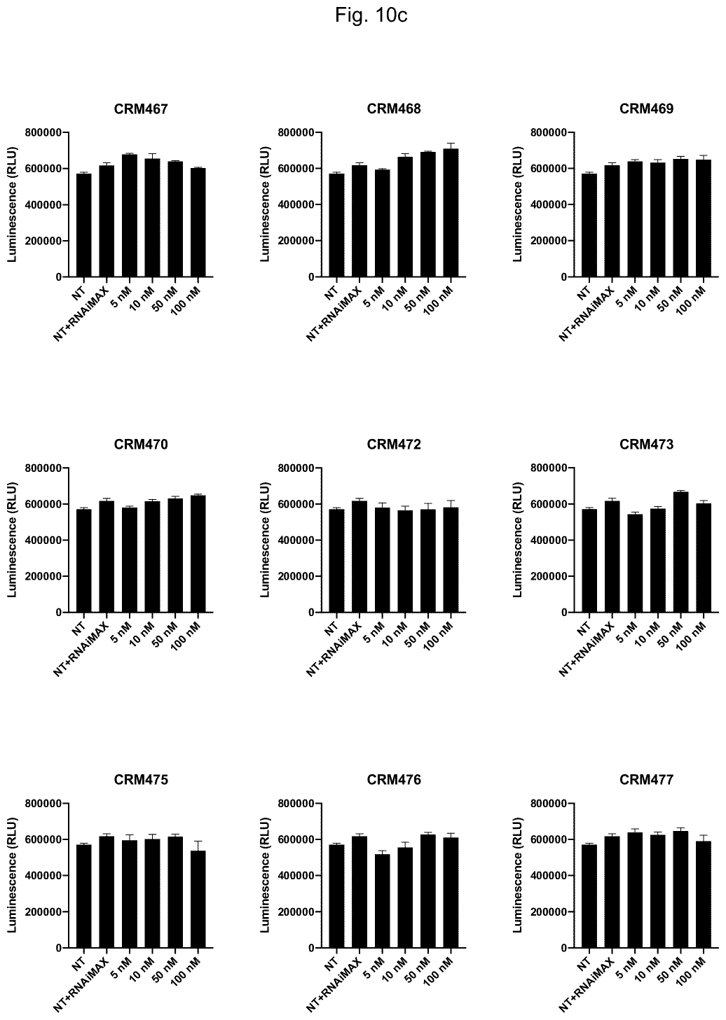

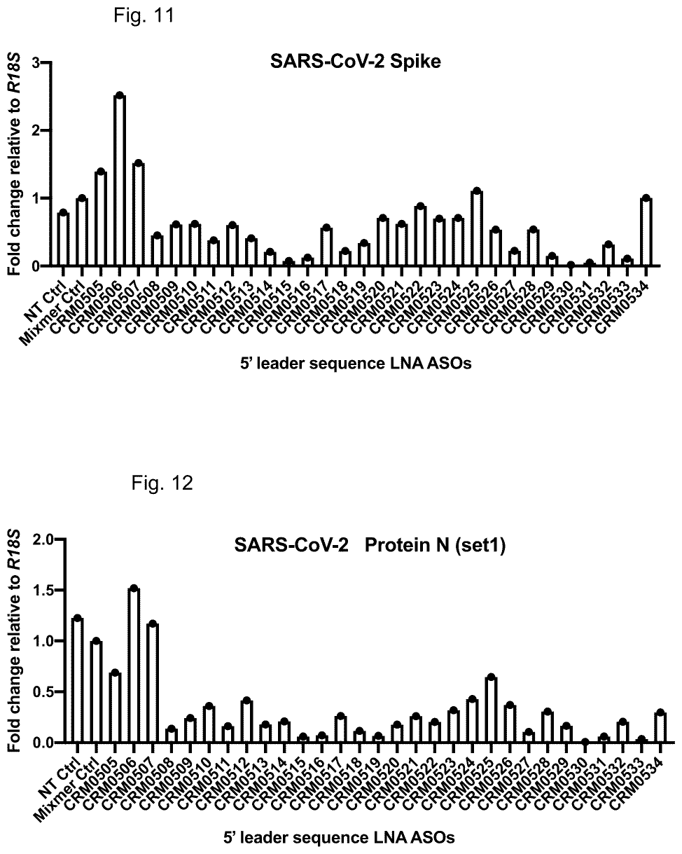

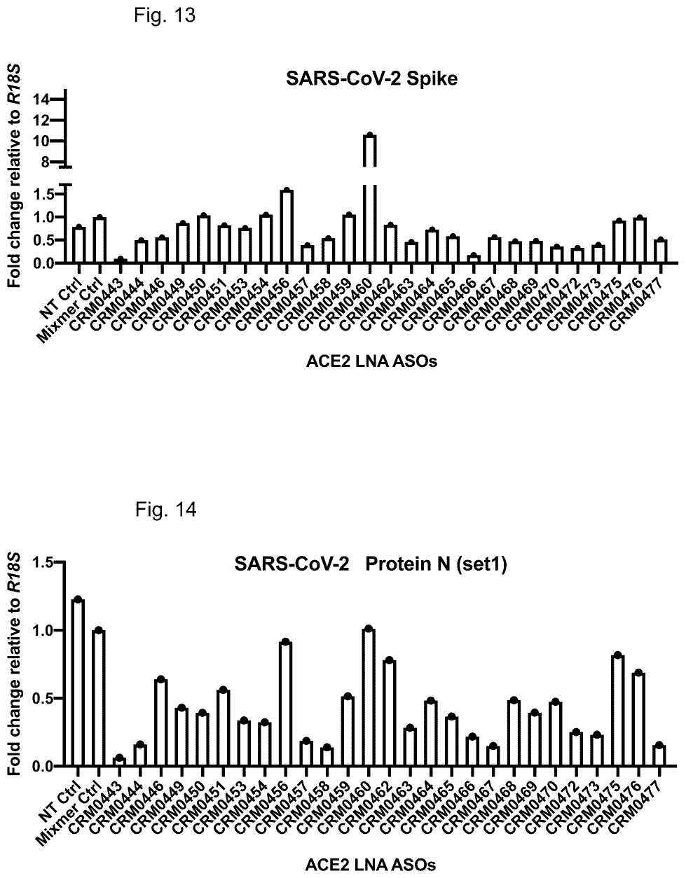

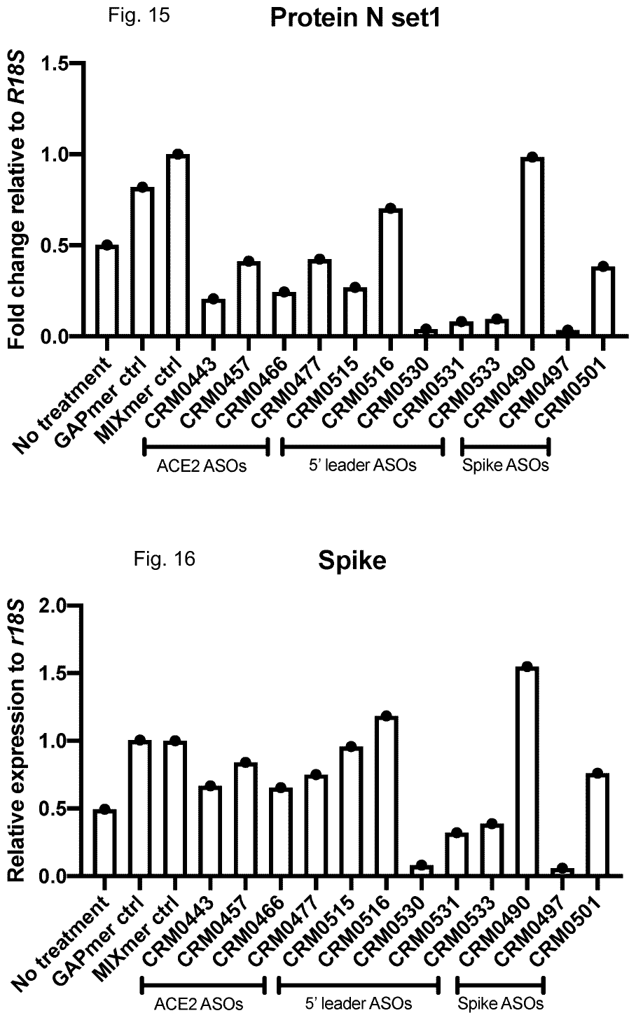

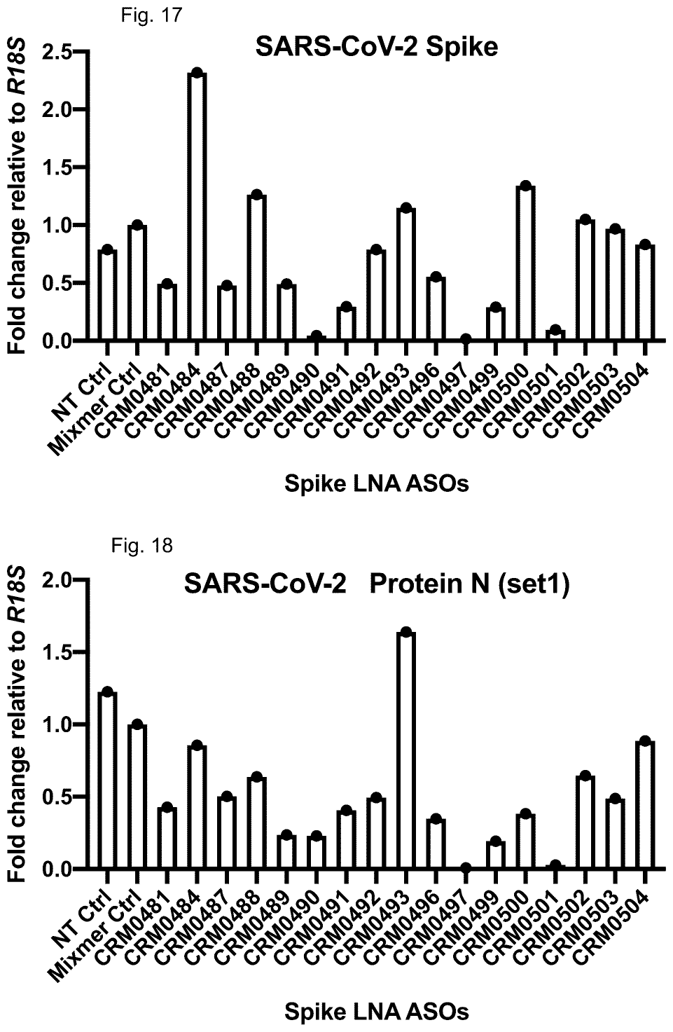

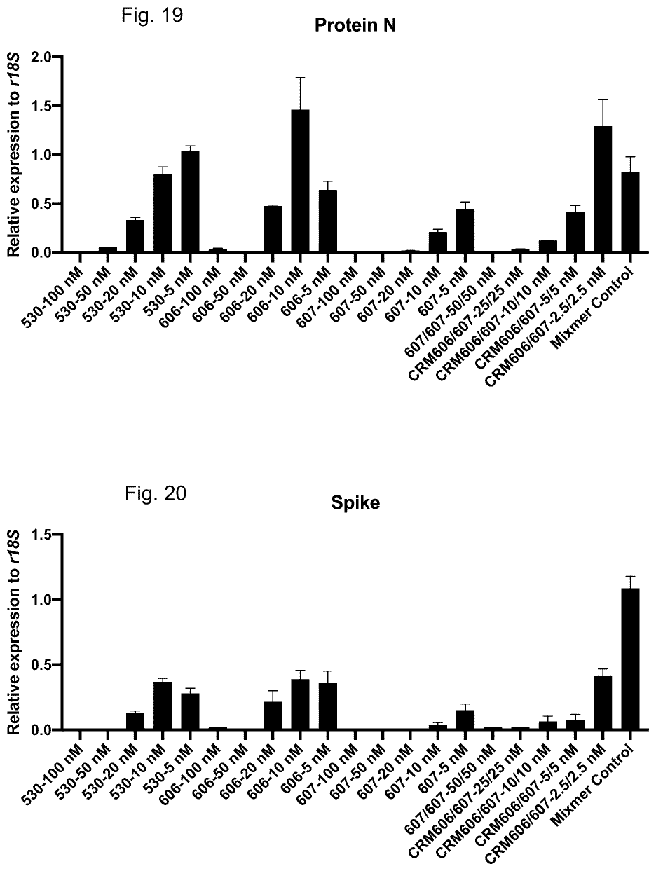

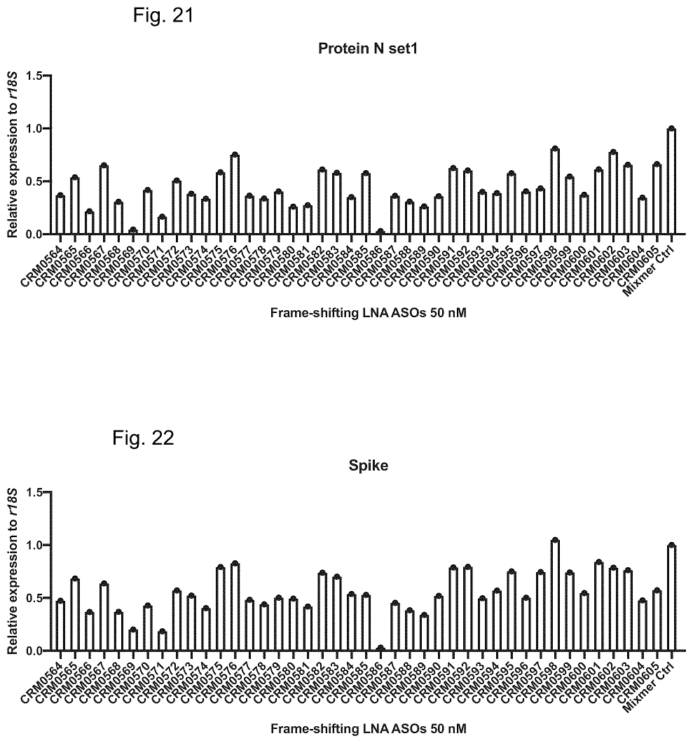

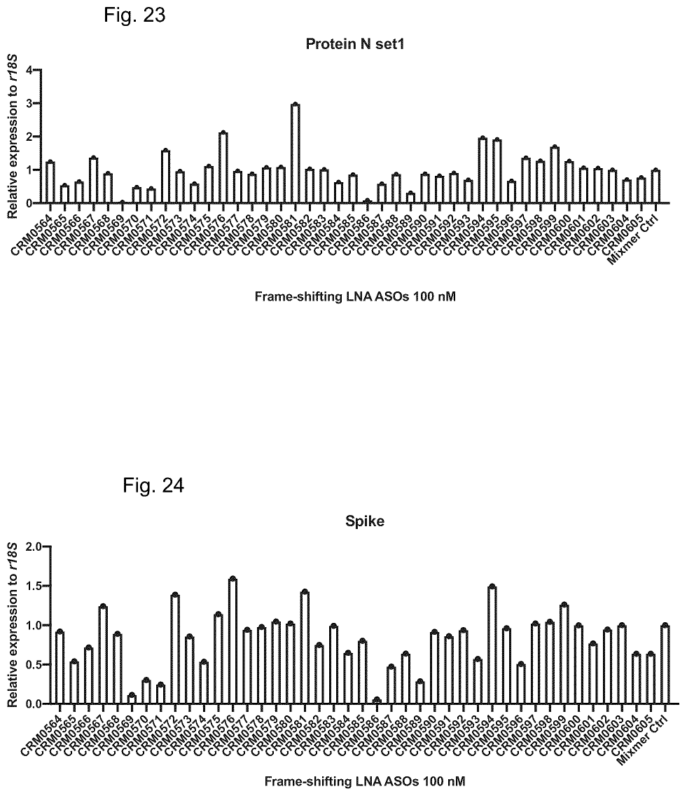

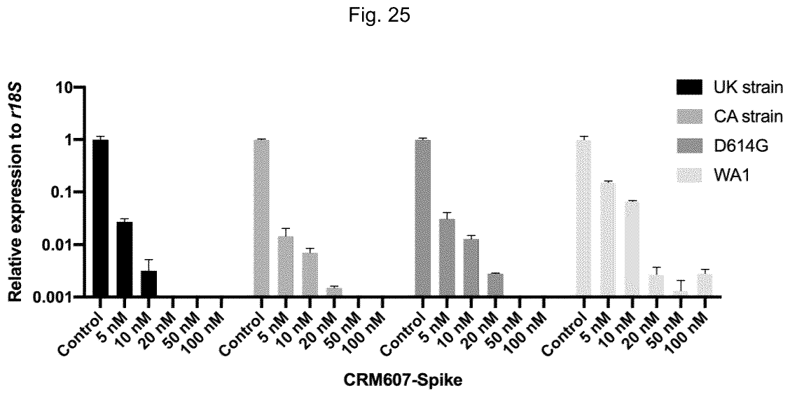

To further evaluate the effect of 5′-ASO #26, we first confirmed that 5′-ASO #26 repressed viral replication in vivo in a dose-dependent manner (FIG. 6b). To explore the optimal treatment time course, we assessed the efficacy of 5′-ASO #26 viral repression in pre-infection and post-infection treatment regimens (FIG. 4a). We found that the strongest viral repressive effect was observed in the Prophylactic #2 group (FIG. 4a), in which the mice were treated with LNA ASO for four days, followed by infection 24 hrs later. Viral repression was also validated by staining of Protein N in mouse lung tissues (FIG. 8a). Notably, a prophylactic effect was still observed one week after LNA ASO treatment (Prophylactic #1, FIG. 4a). Starting LNA ASO treatment at 6 hpi also revealed a moderate repressive effect, whereas beginning treatment from 1 dpi only led to a very marginal repressive effect in mice (Treatment #3 and #4, FIG. 4a). These results show that 5′-ASO #26 exhibits potent effects as a prophylactic therapeutic. The diminished ability of late post-infection treatment of 5′-ASO #26 to inhibit viral replication may be a result of rapidly accumulating sub-genomic viral RNAs saturating the amount of administered LNA ASO in the lung due to the very large dose of virus used to inoculate the mice. Modifications of the LNA ASO (such as lipid-conjugation^{28,29,30}) may be used to further promote cellular uptake of LNA ASO in lung, improving the effect of post-infection LNA ASO treatment. Direct delivery of the LNA ASO to the lung via nebulizer may also be used to improve post-infection therapeutic effect.

Because the 5′ leader sequence of SARS-CoV-2 is highly conserved and as 5′-ASO #26 does not target Spike, we predicted that 5′-ASO #26 should also be able to repress the replication of SARS-CoV-2 variant strains. Therefore, we tested several reported variants with 5′-ASO #26 in cell-based assays. Our results showed that regardless of the mutations, 5′-ASO #26 exhibits potent repressive activity on viral replication of multiple SARS-CoV-2 variants, including B.1.351, D614G, B.1.1.7, and B.1.427 (FIG. 4b and FIG. 8b). We further tested the effect of 5′-ASO #26 in vivo to repress the B.1.427 and B.1.1.7 strains in K18-hACE2 mice following the same schedule showed in FIG. 3a. We performed both TCID₅₀ assays and RT-qPCR of lung homogenates to assess viral titer and observed that 5′-ASO #26 can also block the viral replication of both variant strains in vivo (FIG. 4c, 4e and FIG. 8c). Although mice were inoculated with same amount of variant strains, the in vivo replication rate of B.1.427 was significantly lower than that of B.1.351, however the viral titers of both variant strains were repressed to a similar degree by LNA ASO treatment (FIGS. 4c and 4e). Of note, similar to animals infected with the WA1 strain, the B.1.351 strain did not induce weight loss at 4 dpi (FIG. 4d). However, we noticed that there was a significant weight loss induced by B.1.427 strain infection from 3 dpi in the saline-treated group (FIG. 4f). In contrast, the LNA ASO-treated mice did not exhibit significant weight loss after B.1.427 strain infection (FIG. 4f), indicating that the viral repressive effect of LNA ASO treatment is still able to prevent the progression of SARS-CoV-2-induced pathologies.

Antisense therapy is currently used in clinical treatment for a range of different diseases, including cytomegalovirus retinitis (Fomivirsen³¹), Duchenne muscular dystrophy (Eteplirsen³²), and Spinal Muscular Atrophy (Nusinersen)³³. Here we show for the first time that inhaled LNA ASO treatment represents a promising therapeutic strategy for virus-induced respiratory diseases such as COVID-19. Our study demonstrated that naked LNA ASOs delivered intranasally in saline exhibit potent efficacy in vivo, and no formulation is necessary to achieve therapeutic effect. Considering the relatively small-sized genomes of RNA viruses and the ability to rapidly determine the sequence of any viral genome by next-generation sequencing (NGS) techniques, the design and screening of anti-viral LNA ASOs can be very fast and efficient, allowing for a rapid response to other global health crises posed by emerging viral threats in the future. The apparent ability of single-stranded RNA viruses to accumulate immune-evading mutations presents great challenges for vaccine and therapeutics development. Of note, LNA ASOs can overcome the challenge of mutations due to the ability to design sequences specifically targeted to highly conserved and critical regulatory regions of the viral genome. Additionally, LNA ASO cocktails targeting multiple essential genomic regions of viruses may further increase the efficacy of LNA ASOs as therapeutic candidates to overcome viral evasion mutations. This example provides proof of principle by demonstrating an inhaled LNA ASO targeting the 5′ leader sequence as a viable therapeutic approach for preventing or treating SARS-CoV-2 infections, including those caused by variants of concern, indicating that LNA ASOs can be deployed for the treatment of COVID-19.

FIGS. 1a-e. In vitro screening of LNA ASOs targeting SARS-CoV-2. a) Schematic representation of the genome structure and the regions targeted by LNA ASOs. b) and c) The SARS-CoV-2 WA1 strain-infected Huh-7 cells treated with LNA ASO (100 nM) and cell culture medium were collected at 48 hpi. Viral RNA levels were analyzed by RT-qPCR for LNA ASO screening. Each LNA ASO was tested in duplicate. d) Dose-dependent effects of 5′-ASO #26 were evaluated in infected Huh-7 cells with increasing doses of the LNA ASO (as indicated) by RT-qPCR. e) The infectious virus was measured by TCID₅₀ assay. The infected Huh-7 cells with different doses of LNA ASO treatment were collected at 48 hpi. For d) and e), one-way ANOVA with Dunnett's test was used to determine significance (** P<0.01, *** P<0.001, **** P<0.0001, ns, not significant).

FIGS. 2a-c. 5′-ASO #26 disrupts the stem-loop structure of SL1. a) Scatter plots comparing 5′-leader structures in vitro in different conditions. Comparison of DMS structure signals between the control LNA ASO replicates (left top), and control verses titration with 5′-ASO #26 at 0.1×, 0.45×, 0.7×, 1× and 10× molar ratio of 5′-ASO #26 to the 5′-leader RNA. b) Scatter plots comparing 5′-leader structures in infected Huh-7 cells at different time points. Comparison of DMS signals between samples transfected with control LNA ASO and with 5′-ASO #26 at 6 and 12 dpi. For a) and b) blue dotted line indicates the fitted regression line. Every dot represents an adenine or cytosine in the 5′leader. Pink dots represent adenines or cytosines in the 5′-ASO #26 targeting region. Orange dots represent nucleotides 26, 27 and 28 of the 5′ leader. c) Structural model of SL1 predicted from the DMS-MaPseq of in vitro-transcribed 5′leader RNA with the addition of control LNA ASO (left) and 5′-ASO #26 (right). Nucleotides are color-coded by normalized DMS signal. The 5′-ASO #26-binding site is highlighted with a pink frame; nucleotides 26, 27 and 28 are highlighted with an orange frame; PCR primer binding site, where DMS information is unavailable, is highlighted with a grey frame.

FIGS. 3a-f. Once-daily treatment of 5′-ASO #26 represses the generation of infectious virus in mice. a) Treatment course of once-daily 5′-ASO #26 administration. The red arrow indicates the inoculation of 1×10⁴ TCID50 SARS-CoV-2 intranasally in mice. Mice were treated once-daily with saline (vehicle) or 400 μg LNA ASO (dissolved in saline) and hamsters were treated once-daily with saline (vehicle) or 600 μg LNA ASO (dissolved in saline). Treatment on the day of infection was carried out at 6 hpi. b) The viral burden in lungs of mice treated with Saline (N=5) or LNA ASO (N=5) was measured by TCID₅₀ assay using lung homogenates. c) The levels of viral RNAs encoding Spike and nucleocapsid protein (Protein N) were measured in mouse lungs by RT-qPCR. For b) and c), center line, median; box limits, upper and lower quartiles; plot limits, maximum and minimum in the boxplot. Student t-test was used to determine significance (* P<0.05, **** P<0.0001). d) IHC staining of SARS-CoV-2 Protein N in all infected K18-hACE2 mice with or without LNA ASO treatment. Scale bar=2 mm e) Expression changes of SARS-CoV-2 infection-upregulated genes in Saline- and LNA ASO-treated groups. Columns represent samples and rows represent genes. Colors indicate expression levels (log 2 RPKM) relative to average expression across all samples. f) Gene set enrichment analysis (GSEA) of Hallmark gene sets enriched in lungs of Saline- or LNA ASO-treated mice. Terms were ranked by the false discovery rate (q value).

FIGS. 4a-f. Prophylaxis and treatment of SARS-CoV-2 strains with 5′-ASO #26. a) Mice were administered with different treatment regimens of 5′-ASO #26 as indicated. Treatment on the day of infection was carried out at 6 hpi. The viral burden in lungs of mice in each group (N=5) was measured by TCID₅₀ assay using lung homogenates. One-way ANOVA with Dunnett's test was used to determine significance (* P<0.05). b) Dose-dependent effects of 5′-ASO #26 on inhibition of viral replication of SARS-CoV-2 WA1 and B.1.351 strains were evaluated in infected Huh-7 cells by RT-qPCR. One-way ANOVA with Dunnett's test was used to determine significance (** P<0.01, **** P<0.0001). c) and e) The viral burden in lungs of mice treated with Saline (N=5) and 5′-ASO #26 (N=5) was measured by TCID₅₀ assay using lung homogenates. Student t-test was used to determine significance (**** P<0.0001). d) and f) Weight change was monitored (N=5, symbols represent mean±SD). Two-way ANOVA was used to determine significance (** P<0.01, **** P<0.0001). For a), c) and e), center line, median; box limits, upper and lower quartiles; plot limits, maximum and minimum in the boxplot.

FIGS. 5a-b. In vitro screening of LNA ASOs targeting SARS-CoV-2. a) and b) Infected Huh-7 cells were treated with LNA ASO (100 nM) and cell culture media was collected at 48 hpi. Viral RNA levels were analyzed by RT-qPCR. Each LNA ASO was tested in duplicate. One-way ANOVA with Dunnett's test was used to determine significance (**** P<0.0001).

FIGS. 6a-e. Physiological and histological analysis of LNA ASO-treated K18-hACE2 mice. a) Weight change of mice was monitored over the treatment/infection course (N=5 in each group, symbols represent mean±SD). b) LNA ASO dose-dependence efficacy testing in mice. Mice were administered with different doses of 5′-ASO #26 in 40 μl saline as indicated. The viral burden in lungs of mice in each group (N=3) were measured by TCID₅₀ assay using lung homogenates. c) Representative images of hematoxylin and eosin (H&E) staining of lung sections and immunohistochemistry (IHC) staining of CD3 and B220 in infected K18-hACE2 mice with Saline (N=5) or 5′-ASO #26 treatment (N=5). Scale bar=500 μm. d) The viral burden in lungs of hamsters treated with Saline (N=5) or 5′-ASO #26 (N=5) was measured by TCID₅₀ assay using lung homogenates. Center line, median; box limits, upper and lower quartiles; plot limits, maximum and minimum in the boxplot. e) Weight change of hamsters was monitored over the treatment/infection course (N=5 in each group, symbols represent mean±SD). For d) Student t-test was used to determine significance (*** P<0.001) and for a) and e), Two-way ANOVA was used to determine significance.

FIGS. 7a-e. Evaluating the in vivo effects of 5′-ASO #26 in K18-hACE2 mice. a) Percentage of human/viral sequencing reads in RNA-seq data of Saline- and LNA ASO-treated mouse lung. Reads mapped to the human genome marked in gray and reads mapped to virus marked in red. b) Expression change of SARS-CoV-2 infection-downregulated genes in Saline- and LNA ASO-treated group. Infection. c) GSEA of REACTOME gene sets enriched among upregulated genes in lungs of Saline-treated mice. Terms were ranked by the false discovery rate (q value). d) GSEA plot and heatmap of significantly upregulated genes enriched in cholesterol homeostasis pathway in ASO-treated mice. e) Heatmaps of significantly upregulated and downregulated (>2-fold, FDR<0.01) genes at day 4 of SARS-CoV-2 infection. For b), d) and e) Columns represent samples and rows represent genes. Colors indicate gene expression levels (log 2 RPKM) relative to average expression across all samples.

FIGS. 8a-c. 5′-ASO #26 is capable of repressing the replication of SARS-CoV-2 variant strains. a) Representative images of IHC staining of SARS-CoV-2 nucleocapsid protein (Protein N) in Saline (N=5) or Prophylactic #2 (N=5) regimen groups. Scale bar=2 mm b) Dose-dependent efficacy of 5′-ASO #26 in repressing replication of SARS-CoV-2 variant strains was evaluated in infected Huh-7 cells with increasing doses of 5′-ASO #26 by RT-qPCR. One-way ANOVA with Dunnett's test was used to determine significance (*** P<0.001, **** P<0.0001, #no detection). c) Viral RNA levels in mouse lungs were analyzed by RT-qPCR. Center line, median; box limits, upper and lower quartiles; plot limits, maximum and minimum in the boxplot. Student t-test was used to determine significance (* P<0.05, **** P<0.0001).

Methods

Cell culture and viruses. Huh7 and Vero E6 cells were cultured in Dulbecco's Modified Eagle Medium (DMEM) supplemented with 10% fetal bovine serum (FBS) and 1% Penicillin/Streptomycin. Cells were maintained at 37° C. in 5% CO 2. Transfection in Huh7 cells was performed with Lipofectamine 3000 reagent (Thermo Fisher). Cells were infected by SARS-CoV-2 viruses 12 hrs after transfection. The 2019n-CoV/USA_WA1/2020 isolate of SARS-CoV-2 was obtained from the US Centers for Disease Control and Prevention. Infectious stocks were produced by inoculating Vero E6 cells and collecting the cell culture media upon observation of cytopathic effect; debris were removed by centrifugation and passage through a 0.22 μm filter. The supernatant was then aliquoted and stored at −80° C. D614G, B.1.427 and B.1.1.7 strains were kind gifts from Dr. Mary Kate Morris at the California Department of Public Health (CDPH).

Locked nucleic acid antisense oligonucleotides (LNA ASOs). LNA ASOs were purchased from Integrated DNA Technologies (IDT). For the screening, small scale synthesis (100 nmole) of all LNA ASOs was followed by standard desalting purification. For the animal trials, large scale synthesis (250 mg) of the 5′-ASO #26 LNA ASO was followed by HPLC purification and endotoxin test.

Animals. C57BL/6J and K18-hACE2 [B6.Cg-Tg(K18-ACE2)₂Prlmn/J] mice were purchased from the Jackson Laboratory and male golden Syrian hamsters at 4-5 weeks old were obtained from Charles River Labs (Strain Code: 049). K18-hACE2 mice or hamsters were anesthetized using isoflurane and inoculated with 1×10⁴ TCID₅₀ (for mice) or 10 TCID₅₀ (for hamsters) of SARS-CoV-2 intranasally. For the treatment, saline (40 μl) or 5′-ASO #26 (indicated amount of LNA ASO in 40 μl saline) was administered once-daily intranasally. Mice and hamsters were sacrificed at 4 days post infection (dpi). The left lung lobe was collected and lysed for fifty-percent tissue culture infective dose (TCID₅₀) assay, the inferior lobe was collected for RNA extraction and the post-caval lobe was collected for histological analysis.

Fifty-percent tissue culture infective dose (TCID₅₀) assay. Virus viability and titers were evaluated in TCID₅₀ assay within Vero E6 cells. Briefly, ten thousand cells were plated in each well in 96-well plates and cultured at 37° C. overnight. Medium from SARS-CoV-2-infected cells or lysates from mouse lungs were used for ten-fold serial dilution with DMEM and added to the 96-well plates of Vero E6 cells. The plates were observed for cytopathic effect (CPE) after 3 days of culturing. The TCID₅₀ results were calculated using the Spearman and Karber method³⁴.

RNA extraction and real-time quantitative PCR (RT-qPCR). Infected Huh-7 cells (with or without medium) or mouse lung tissues were lysed in DNA/RNA shield reagent (Zymo Research) and total RNA was extracted by using RNeasy kit (Qiagen) according to the manufacturer's protocol. cDNA was prepared by iScript™ Reverse Transcription Supermix (BioRAD) and qPCR was performed with Fast SYBR™ Green Master Mix (Thermo Fisher) and the reaction was run on the QuantStudio6 System (Applied Biosystems). mRNA levels were normalized to that of r185. qPCR primer sets are as follow: SARS-CoV-2 Protein N: Fw 5′-GACCCCAAAATCAGCGAA AT-3′ (SEQ ID NO: 212) and Rv 5′-TCTGGTTACTGCCAGTTGAATCTG-3′ (SEQ ID NO: 213), SARS-CoV-2 Spike: Fw 5′-GTCCTTCCCTCAGTCAGCAC-3′ (SEQ ID NO: 214) and Rv 5′-ATGGCAGGAGCAGTTGTGAA-3′ (SEQ ID NO: 215), Human r185: Fw 5′-GTAACCCGTTGAACCCCATT-3′ (SEQ ID NO: 216) and Rv 5′-CCATCCAATCGGTAGTAGCG-3′ (SEQ ID NO: 217), Mouse r185: Fw 5′-GCAATTATTCCCCATGAACG-3′ (SEQ ID NO: 218) and Rv 5′-GGCCTCACTAAACCATCCAA-3′ (SEQ ID NO: 219).

RNA-sequencing. cDNA libraries were constructed from 500 ng of total RNA from Huh-7 or lung tissues of mice according to the manufacturer's protocol of Stranded mRNA-seq kit (KAPA). Briefly, mRNA was captured and fragmentized to 100-300 bp. library construction was performed undergoing end repair, A tailing, ligation of unique dual-indexed adapters (KAPA) and amplification of 10 cycles to incorporate unique dual index sequences. Libraries were sequenced on the NovaSeq 6000 (Novogene) targeting 40 million read pairs and extending 150 cycles with paired end reads. The data have been deposited in NCBI's Gene Expression Omnibus and are accessible through GEO Series accession number GSE174382. The published RNA-seq data of infected K18-hACE2 at 0 dpi and 4 dpi were obtained from GSE154104. STAR aligner³⁵ was used to map sequencing reads to transcripts in the mouse mm10 reference genome. Read counts for individual transcripts were produced with HTSeq-count³⁶, followed by the estimation of expression values and detection of differentially expressed transcripts using EdgeR³⁷. Differentially expressed genes were defined by at least 2-fold change with FDR less than 0.01.

DMS modification of in vitro-transcribed RNA. gBlock containing the first 3,000 nucleotides of the SARS-CoV-2 genome (2019-nCoV/USA-WA1/2020) was obtained from IDT. The gBlock was amplified by PCR with a forward primer that contained the T7 promoter sequence (5′-TAATACGACTCACTATAGGGATTAAAGGTTTATACCTTCCCAGGTAAC-3′) (SEQ ID NO: 220) and the reverse primer (5′-TCGTTGAAACCAGGGACAAG-3′) (SEQ ID NO: 221). The PCR product was used as the template for T7 Megascript in vitro transcription (ThermoFisher Scientific) according to manufacturer's instructions. Next, 1 μl of Turbo DNase I (ThermoFisher Scientific) was added and incubated at 37° C. for 15 mM The RNA was purified using RNA Clean and Concentrator™-5 (Zymo). Between 1-2 μg RNA was denatured at 95° C. for 1 min. Denatured RNA was refolded in the presence of 2 μM of LNA ASO by incubating the mixture in 340 mM sodium cacodylate buffer (Electron Microscopy Sciences) and 5 mM MgCl²⁺, such that the volume was 97.5 for 20 mM at 37° C. Then, 2.5% DMS (Millipore-Sigma) was added and incubated for 5 mM at 37° C. whole shaking at 800 r.p.m. on a thermomixer. Subsequently, DMS was neutralized by adding 60 μl β-mercaptoethanol (Millipore-Sigma). The RNA was precipitated by incubating in 3 μl (45 μg) glycoblue coprecipitant (Invitrogen), 18 μl 3M sodium acetate and 700 μl ethanol between 1 h and overnight at −80° C., followed by centrifugation at max speed for 45 mM in 4° C. The RNA was washed with 700 μl ice cold 75% ethanol and centrifuged for 5 mM RNA was resuspended in 10 μl water.

DMS modification of infected Huh-7 cells with ASO treatment. Huh-7 cells were transfected with LNA ASO (50 nM) 12 h before the infection. After infection of SARS-CoV-2 (MOI 0.05), cells were cultured in 6-well plates with 2 ml of media. Then, 2.5% DMS was added to cells and incubated for 3 mM at 37° C. Subsequently, after careful removal of the media, DMS was neutralized by adding 20 ml of chilled 10% 0-mercaptoethanol in PBS. The cell pellets were washed once with chilled PBS and collected for RNA extraction.

rRNA subtraction of total cellular RNA from DMS-treated cells. Between 3-5 μg RNA per sample was used as the input for rRNA subtraction. First, equal amount of rRNA pooled oligonucleotides were added and incubated in hybridization buffer (200 mM NaCl, 100 mM Tris-HCl, pH 7.4) in a final volume of 60 The samples were denatured for 2 min at 95° C., followed by a reduction of 0.1° C./s until the reaction reached 45° C. 3-5 μl Hybridase™ Thermostable RNase H (Lucigen) and 7 μl 10×RNase H buffer preheated to 45° C. was added. The samples were incubated at 45° C. for 30 mM The RNA was purified using RNA Clean and Concentrator™-5 kit and eluted in 42 μl water. Then, 5 μl Turbo DNase buffer and 3 μl Turbo DNase (ThermoFisher Scientific) were added to each sample and incubated for 30 mM at 37° C. The RNA was purified using RNA Clean and Concentrator™-5 kit and eluted in 10 μl water.

RT-PCR and sequencing of DMS-modified RNA. To reverse transcribe, rRNA-depleted total RNA or in vitro-transcribed RNA purified from the previous steps was added to 4 μl 5×FS buffer, 1 μl dNTP, 1 μl of 0.1 M DTT, 1 μl RNase Out, 1 μl of 10 μM reverse primer (5′-TCGTTGAAACCAGGGACAAG-3′) (SEQ ID NO:221) and 1 μl TGIRT-III (Ingex). The reaction was incubated for 1.5 h at 60° C. Then, to degrade the RNA, 1 μl of 4 M NaOH was added and incubated for 3 mM at 95° C. The cDNA was purified in 10 μl water using the Oligo Clean and Concentrator™ kit (Zymo). Next, 1 μl of cDNA was amplified using Advantage HF 2 DNA polymerase (Takara) for 25-30 cycles according to the manufacturer's instructions (Fw 5′-GGGATTAAAGGTTTATACCTTCCC-3′ (SEQ ID NO:222) and Rv 5′-TCGTTGAAACCAGGGACAAG-3′) (SEQ ID NO: 221). The PCR product was purified using E-Gel™ SizeSelect™ II 2% agarose gel (Invitrogen). RNA-seq library for 300 bp insert size was constructed following the manufacturer's instructions (NEBNext Ultra™ II DNA Library Prep Kit). The library was loaded on iSeq-100 Sequencing flow cell with iSeq-100 High-throughput sequencing kit and library was run on iSeq-100 (paired-end run, 151×151 cycles).

Immunohistochemistry (IHC) and hematoxylin and eosin (H&E) staining. Histology was performed by HistoWiz Inc. using a Standard Operating Procedure and fully automated workflow. Samples were processed, embedded in paraffin, and sectioned at 4 μm. Immunohistochemistry was performed on a Bond Rx autostainer (Leica Biosystems) with enzyme treatment (1:1000) using standard protocols. Antibodies used were rabbit monoclonal CD3 primary antibody (Abcam, ab16669, 1:100), rabbit monoclonal B220 primary antibody (Novus, NB100-77420, 1:10000), rabbit monoclonal SARS-CoV-2 (COVID-19) nucleocapsid primary antibody (GeneTex, GTX635686, 1:8000) and rabbit anti-rat secondary (Vector, 1:100). Bond Polymer Refine Detection (Leica Biosystems) was used according to the manufacturer's protocol. After staining, sections were dehydrated and film coverslipped using a TissueTek-Prisma and Coverslipper (Sakura). Whole slide scanning (40×) was performed on an Aperio AT2 (Leica Biosystems).

Statistical analysis. Data are presented as mean values, and error bars represent SD. Data analysis was performed using GraphPad Prism 8. Data were analyzed using unpaired t-test; one-way or two-way ANOVA followed by Turkey or Dunnett test as indicated. P value <0.05 was considered as statistically significant.

### REFERENCES

1. Kuzmina, A. et al. SARS-CoV-2 spike variants exhibit differential infectivity and neutralization resistance to convalescent or post-vaccination sera. Cell Host Microbe 29, 522-528.e2 (2021) doi:10.1016/j.chom.2021.03.008.
2. de Vries, R. D. et al. Intranasal fusion inhibitory lipopeptide prevents direct-contact SARS-CoV-2 transmission in ferrets. Science (80-.). 371, 1379-1382 (2021) doi:10.1126/science.abf4896originally.
3. Fontanet, A. et al. SARS-CoV-2 variants and ending the COVID-19 pandemic. The Lancet 397, 952-954 (2021) doi:10.1016/50140-6736(21)00370-6.
4. Hagedorn, P. H. et al. Locked nucleic acid: modality, diversity, and drug discovery. Drug Discovery Today 23, 101-114 (2018) doi:10.1016/j.drudis.2017.09.018.
5. Zhu, N. et al. A Novel Coronavirus from Patients with Pneumonia in China, 2019. N. Engl. J. Med. 382, 727-733 (2020) doi:10.1056/nejmoa2001017.
6. Petersen, E. et al. Comparing SARS-CoV-2 with SARS-CoV and influenza pandemics. Lancet Infect. Dis. 20, e238-e244 (2020) doi:10.1016/S1473-3099(20)30484-9.
7. Wouters, O. J. et al. Challenges in ensuring global access to COVID-19 vaccines: production, affordability, allocation, and deployment. The Lancet 397, 1023-1034 (2021) doi:10.1016/S0140-6736(21)00306-8.
8. Coutinho, R. M. et al. Model-based estimation of transmissibility and reinfection of SARS-CoV-2 P.1 variant. medRxiv 2021.03.03.21252706 (2021) doi:10.1101/2021.03.03.21252706.
9. Wang, L. et al. Profiling and characterization of SARS-CoV-2 mutants' infectivity and antigenicity. Signal Transduct. Target. Ther. 5, 1-2 (2020) doi:10.1038/s41392-020-00302-8.
10. Huang, Y. et al. Structural and functional properties of SARS-CoV-2 spike protein: potential antivirus drug development for COVID-19. Acta Pharmacol. Sin. 41, 1141-1149 (2020) doi:10.1038/s41401-020-0485-4.
11. Davies, N. G. et al. Increased mortality in community-tested cases of SARS-CoV-2 lineage B.1.1.7. Nature 593, (2021) doi:10.1038/s41586-021-03426-1.
12. Roberts, T. C. et al. Advances in oligonucleotide drug delivery. Nature Reviews Drug Discovery 19, 673-694 (2020) doi:10.1038/s41573-020-0075-7.
13. Wengel, J. et al. LNA (Locked Nucleic Acid). Nucleosides and Nucleotides 18, 1365-1370 (1999) doi:10.1080/07328319908044718.
14. Wang, G. et al. Conformationally locked nucleosides. Synthesis and hybridization properties of oligodeoxynucleotides containing 2′,4′-C-bridged 2′-deoxynucleosides. Bioorganic Med. Chem. Lett. 9, 1147-1150 (1999) doi:10.1016/S0960-894X(99)00146-8.
15. Eckstein, F. Phosphorothioates, essential components of therapeutic oligonucleotides. Nucleic Acid Ther. 24, 374-387 (2014) doi:10.1089/nat.2014.0506.
16. Dutta, N. K. et al. The Nucleocapsid Protein of SARS-CoV-2: a Target for Vaccine Development. J. Virol. 94, (2020) doi:10.1128/jvi.00647-20.
17. Tidu, A. et al. The viral protein NSP1 acts as a ribosome gatekeeper for shutting down host translation and fostering SARS-CoV-2 translation. RNA 27, 253-264 (2021) doi:10.1261/rna.078121.120.
18. Kim, D. et al. The Architecture of SARS-CoV-2 Transcriptome. Cell 181, 914-921.e10 (2020) doi:10.1016/j.cell.2020.04.011.
19. Kelly, J. A. et al. Structural and functional conservation of the programmed—1 ribosomal frameshift signal of SARS-CoV-2. bioRxiv (2020) doi:10.1101/2020.03.13.991083.
20. Chen, S. C. et al. Group-specific structural features of the 5′-proximal sequences of coronavirus genomic RNAs. Virology 401, 29-41 (2010) doi:10.1016/j.virol.2010.02.007.
21. Lan, T. C. T. et al. Insights into the secondary structural ensembles of the full SARS-CoV-2 RNA genome in infected cells. bioRxiv 2020.06.29.178343 (2021) doi:10.1101/2020.06.29.178343.
22. Zubradt, M. et al. DMS-MaPseq for genome-wide or targeted RNA structure probing in vivo. Nat. Methods 14, 75-82 (2016) doi:10.1038/nmeth.4057.
23. Winkler, E. S. et al. SARS-CoV-2 infection of human ACE2-transgenic mice causes severe lung inflammation and impaired function. Nat. Immunol. 21, 1327-1335 (2020) doi:10.1038/s41590-020-0778-2.
24. Sia, S. F. et al. Pathogenesis and transmission of SARS-CoV-2 in golden hamsters. Nature 583, 834-838 (2020) doi:10.1038/s41586-020-2342-5.
25. Wang, R. et al. Genetic Screens Identify Host Factors for SARS-CoV-2 and Common Cold Coronaviruses. Cell 184, 106-119.e14 (2021) doi:10.1016/j.cell.2020.12.004.
26. Schneider, W. M. et al. Genome-Scale Identification of SARS-CoV-2 and Pan-coronavirus Host Factor Networks. Cell 184, 120-132.e14 (2021) doi:10.1016/j.cell.2020.12.006.
27. Hu, X. et al. Low Serum Cholesterol Level Among Patients with COVID-19 Infection in Wenzhou, China. SSRN (2020) doi:10.2139/ssm.3544826.
28. Wang, S. et al. Lipid Conjugates Enhance Endosomal Release of Antisense Oligonucleotides into Cells. Nucleic Acid Ther. 29, 245-255 (2019) doi:10.1089/nat.2019.0794.
29. Benizri, S. et al. Bioconjugated Oligonucleotides: Recent Developments and Therapeutic Applications. Bioconjugate Chemistry 30, 366-383 (2019) doi:10.1021/acs.bioconjchem.8b00761.
30. Biscans, A. et al. Docosanoic acid conjugation to siRNA enables functional and safe delivery to skeletal and cardiac muscles. Mol. Ther. 29, 1382-1394 (2021) doi:10.1016/j.ymthe.2020.12.023.
31. Grillone, L. et al. Fomivirsen. Kucers Use Antibiot. A Clin. Rev. Antibacterial, Antifung. Antiparasit. Antivir. Drugs, Seventh Ed. 57, 3647-3651 (2017) doi:10.1201/9781315152110.
32. Lim, K. R. Q. et al. Eteplirsen in the treatment of Duchenne muscular dystrophy. Drug Des. Devel. Ther. 11, 533-545 (2017) doi:10.2147/DDDT.S97635.
33. Gidaro, T. et al. Nusinersen treatment of spinal muscular atrophy: current knowledge and existing gaps. Developmental Medicine and Child Neurology 61, 19-24 (2019) doi:10.1111/dmcn.14027.
34. Ramakrishnan, M. A. Determination of 50% endpoint titer using a simple formula. World J. Virol. 5, 85 (2016) doi:10.5501/wjv.v5.i2.85.
35. Dobin, A. et al. STAR: Ultrafast universal RNA-seq aligner. Bioinformatics 29, 15-21 (2013) doi:10.1093/bioinformatics/bts635.
36. Anders, S. et al. HTSeq-A Python framework to work with high-throughput sequencing data. Bioinformatics 31, 166-169 (2015) doi:10.1093/bioinformatics/btu638.
37. Robinson, M. D. et al. edgeR: A Bioconductor package for differential expression analysis of digital gene expression data. Bioinformatics 26, 139-140 (2009) doi:10.1093/bioinformatics/btp616.

Extended Data Table 1. Targeting sites of LNA ASOs used in in vitro screening. To
identify LNA ASOs exhibiting anti-viral efficacy, LNA ASOs targeting 5′ leader
sequences, 5′ UTR region of ORF1a, FSE of ORF1a/b and Spike coding region were
tested in cell-based screening assays in Huh-7 cells.

| LNA ASO ID | Type | Target region SARS-COV-2 | Targeting Site |
|---|---|---|---|
| 5′-ASO#01 | gapmer | 5′ leader sequence | GUUCUCUAAACGAAC |
| | | | (SEQ ID NO: 140) |
| 5′-ASO#02 | gapmer | 5′ leader sequence | AUCUGUUCUCUAAACG |
| | | | (SEQ ID NO: 141) |
| 5′-ASO#03 | gapmer | 5′ leader sequence | GAUCUGUUCUCUAAAC |
| | | | (SEQ ID NO: 142) |
| 5′-ASO#04 | gapmer | 5′ leader sequence | CUUGUAGAUCUGUUCUC |
| | | | (SEQ ID NO: 143) |
| 5′-ASO#05 | gapmer | 5′ leader sequence | CUUGUAGAUCUGUUC |
| | | | (SEQ ID NO: 144) |
| 5′-ASO#06 | gapmer | 5′ leader sequence | GAUCUCUUGUAGAUCUG |
| | | | (SEQ ID NO: 145) |
| 5′-ASO#07 | gapmer | 5′ leader sequence | CGAUCUCUUGUAGAUC |
| | | | (SEQ ID NO: 146) |
| 5′-ASO#08 | gapmer | 5′ leader sequence | AACUUUCGAUCUCUUG |
| | | | (SEQ ID NO: 147) |
| 5′-ASO#09 | gapmer | 5′ leader sequence | GAUCUCUUGUAGAUC |
| | | | (SEQ ID NO: 148) |
| 5′-ASO#10 | gapmer | 5′ leader sequence | ACCAACUUUCGAUCUC |
| | | | (SEQ ID NO: 149) |
| 5′-ASO#11 | gapmer | 5′ leader sequence | AAACCAACCAACUUUC |
| | | | (SEQ ID NO: 150) |
| 5′-ASO#12 | gapmer | 5′ leader sequence | CAGGUAACAAACCAAC |
| | | | (SEQ ID NO: 151) |
| 5′-ASO#13 | gapmer | 5′ leader sequence | AGGUAACAAACCAACC |
| | | | (SEQ ID NO: 152) |
| 5′-ASO#14 | gapmer | 5′ leader sequence | UUCCCAGGUAACAAAC |
| | | | (SEQ ID NO: 153) |
| 5′-ASO#15 | gapmer | 5′ leader sequence | ACCUUCCCAGGUAAC |
| | | | (SEQ ID NO: 154) |
| 5′-ASO#16 | mixmer | 5′ leader sequence | GUUCUCUAAACGAAC |
| | | | (SEQ ID NO: 140) |
| 5′-ASO#17 | mixmer | 5′ leader sequence | AUCUGUUCUCUAAACG |
| | | | (SEQ ID NO: 141) |
| 5′-ASO#18 | mixmer | 5′ leader sequence | GAUCUGUUCUCUAAAC |
| | | | (SEQ ID NO: 142) |
| 5′-ASO#19 | mixmer | 5′ leader sequence | CUUGUAGAUCUGUUCUC |
| | | | (SEQ ID NO: 143) |
| 5′-ASO#20 | mixmer | 5′ leader sequence | CUUGUAGAUCUGUUC |
| | | | (SEQ ID NO: 144) |
| 5′-ASO#21 | mixmer | 5′ leader sequence | GAUCUCUUGUAGAUCUG |
| | | | (SEQ ID NO: 145) |
| 5′-ASO#22 | mixmer | 5′ leader sequence | CGAUCUCUUGUAGAUC |
| | | | (SEQ ID NO: 146) |
| 5′-ASO#23 | mixmer | 5′ leader sequence | AACUUUCGAUCUCUUG |
| | | | (SEQ ID NO: 147) |
| 5′-ASO#24 | mixmer | 5′ leader sequence | CGAUCUCUUGUAGAUC |
| | | | (SEQ ID NO: 146) |
| 5′-ASO#25 | mixmer | 5′ leader sequence | ACCAACUUUCGAUCUC |
| | | | (SEQ ID NO: 149) |
| 5′-ASO#26 | mixmer | 5′ leader sequence | AAACCAACCAACUUUC |
| | | | (SEQ ID NO: 150) |
| 5′-ASO#27 | mixmer | 5′ leader sequence | CAGGUAACAAACCAAC |
| | | | (SEQ ID NO: 151) |
| 5′-ASO#28 | mixmer | 5′ leader sequence | AGGUAACAAACCAACC |
| | | | (SEQ ID NO: 152) |
| 5′-ASO#29 | mixmer | 5′ leader sequence | UUCCCAGGUAACAAAC |
| | | | (SEQ ID NO: 153) |
| 5′-ASO#30 | mixmer | 5′ leader sequence | ACCUUCCCAGGUAAC |
| | | | (SEQ ID NO: 154) |
| S-ASO#01 | gapmer | Spike coding region | AUCAACUUACUCCUACU |
| | | | (SEQ ID NO: 106) |
| S-ASO#02 | gapmer | Spike coding region | CCCUACUUAUUGUUAAU |
| | | | (SEQ ID NO: 98) |
| S-ASO#03 | gapmer | Spike coding region | CAGACUAAUUCUCCUCG |
| | | | (SEQ ID NO: 107) |
| S-ASO#04 | gapmer | Spike coding region | GUCCUAUAUAAUUCCG |
| | | | (SEQ ID NO: 101) |
| S-ASO#05 | gapmer | Spike coding region | UCUGCUUUACUAAUGUC |
| | | | (SEQ ID NO: 102) |
| S-ASO#06 | gapmer | Spike coding region | GGUGUUCUUACUGAGUC |
| | | | (SEQ ID NO: 103) |
| S-ASO#07 | gapmer | Spike coding region | AUGUCCUUCCCUCAGUC |
| | | | (SEQ ID NO: 111) |
| S-ASO#08 | gapmer | Spike coding region | GCUUACUCUAAUAACUC |
| | | | (SEQ ID NO: 108) |
| S-ASO#09 | gapmer | Spike coding region | AGUUUAUGAUCCUUUGC |
| | | | (SEQ ID NO: 113) |
| S-ASO#10 | gapmer | Spike coding region | UGAAUAUGUCUCUCAGC |
| | | | (SEQ ID NO: 99) |
| S-ASO#11 | gapmer | Spike coding region | CAGUGUGUUAAUCUUAC |
| | | | (SEQ ID NO: 97) |
| S-ASO#12 | gapmer | Spike coding region | AAUCAUUACUACAGAC |
| | | | (SEQ ID NO: 112) |
| S-ASO#13 | gapmer | Spike coding region | GAACAAAUACUUCUAAC |
| | | | (SEQ ID NO: 105) |
| S-ASO#14 | gapmer | Spike coding region | UGAGAUUCUUGACAUUA |
| | | | (SEQ ID NO: 104) |
| S-ASO#15 | gapmer | Spike coding region | UGCUAAUCUUGCUGCUA |
| | | | (SEQ ID NO: 110) |
| S-ASO#16 | gapmer | Spike coding region | UGGUGAUAUUGCUGCUA |
| | | | (SEQ ID NO: 109) |
| S-ASO#17 | gapmer | Spike coding region | CACUUGACCCUCUCUCA |
| | | | (SEQ ID NO: 100) |
| U-ASO#01 | mixmer | 5′UTR regions of ORF1a | GAUGGAGAGCCUUGUC |
| | | | (SEQ ID NO: 199) |
| U-ASO#02 | mixmer | 5′UTR regions of ORF1a | GGUGUGACCGAAAGG |
| | | | (SEQ ID NO: 200) |
| U-ASO#03 | mixmer | 5′UTR regions of ORF1a | AUCUAGGUUUCGUCCG |
| | | | (SEQ ID NO: 201) |
| U-ASO#04 | mixmer | 5′UTR regions of ORF1a | CAGCCGAUCAUCAGCAC |
| | | | (SEQ ID NO: 202) |
| U-ASO#05 | mixmer | 5′UTR regions of ORF1a | GGUUUCGUCCGUGUUG |
| | | | (SEQ ID NO: 196) |
| U-ASO#06 | mixmer | 5′UTR regions of ORF1a | CUGCAGGCUGCUUAC |
| | | | (SEQ ID NO: 203) |
| U-ASO#07 | mixmer | 5′UTR regions of ORF1a | GUAACUCGUCUAUCUU |
| | | | (SEQ ID NO: 204) |
| U-ASO#08 | mixmer | 5′UTR regions of ORF1a | CGUUGACAGGACACGA |
| | | | (SEQ ID NO: 197) |
| U-ASO#09 | mixmer | 5′UTR regions of ORF1a | UAAUAACUAAUUACUGU |
| | | | (SEQ ID NO: 198) |
| U-ASO#10 | mixmer | 5′UTR regions of ORF1a | CUCACGCAGUAUAAU |
| | | | (SEQ ID NO: 205) |
| U-ASO#11 | mixmer | 5′UTR regions of ORF1a | CUGCAUGCUUAGUGCA |
| | | | (SEQ ID NO: 206) |
| U-ASO#12 | mixmer | 5′UTR regions of ORF1a | GUGUGGCUGUCACUCGG |
| | | | (SEQ ID NO: 207) |
| U-ASO#13 | mixmer | 5′UTR regions of ORF1a | ACGAACUUUAAAAUCU |
| | | | (SEQ ID NO: 208) |
| U-ASO#14 | mixmer | 5′UTR regions of ORF1a | GUAGAUCUGUUCUCUAA |
| | | | (SEQ ID NO: 209) |
| U-ASO#15 | mixmer | 5′UTR regions of ORF1a | CAACUUUCGAUCUCUU |
| | | | (SEQ ID NO: 210) |
| U-ASO#16 | mixmer | 5′UTR regions of ORF1a | CAGGUAACAAACCAAC |
| | | | (SEQ ID NO: 151) |
| U-ASO#17 | mixmer | 5′UTR regions of ORF1a | AAGGUUUAUACCUUCC |
| | | | (SEQ ID NO: 211) |
| F-ASO#01 | mixmer | FSE region of ORF1a/b | CAAGAAAAGGACGAAG |
| | | | (SEQ ID NO: 157) |
| F-ASO#02 | mixmer | FSE region of ORF1a/b | UAAUUGUUGUCGCUUC |
| | | | (SEQ ID NO: 158) |
| F-ASO#03 | mixmer | FSE region of ORF1a/b | CUAAAUUCCUAAAAAC |
| | | | (SEQ ID NO: 159) |
| F-ASO#04 | mixmer | FSE region of ORF1a/b | AAAGUAGCUGGUUUUG |
| | | | (SEQ ID NO: 160) |
| F-ASO#05 | mixmer | FSE region of ORF1a/b | UGACAUCUACAAUGAU |
| | | | (SEQ ID NO: 161) |
| F-ASO#06 | mixmer | FSE region of ORF1a/b | UCGUAUACAGGGCUUU |
| | | | (SEQ ID NO: 162) |
| F-ASO#07 | mixmer | FSE region of ORF1a/b | GGCACUAGUACUGAUG |
| | | | (SEQ ID NO: 163) |
| F-ASO#08 | mixmer | FSE region of ORF1a/b | UACACCGUGCGGCACA |
| | | | (SEQ ID NO: 164) |
| F-ASO#09 | mixmer | FSE region of ORF1a/b | UAAGUGCAGCCCGUCU |
| | | | (SEQ ID NO: 165) |
| F-ASO#10 | mixmer | FSE region of ORF1a/b | AAACGGGUUUGCGGUG |
| | | | (SEQ ID NO: 166) |
| F-ASO#11 | mixmer | FSE region of ORF1a/b | AUGCACAAUCGUUUUU |
| | | | (SEQ ID NO: 167) |
| F-ASO#12 | mixmer | FSE region of ORF1a/b | AUGCUUCAGUCAGCUG |
| | | | (SEQ ID NO: 168) |
| F-ASO#13 | mixmer | FSE region of ORF1a/b | UCAACUCCGCGAACCC |
| | | | (SEQ ID NO: 169) |
| F-ASO#14 | mixmer | FSE region of ORF1a/b | AUGGCUGUAGUUGUGA |
| | | | (SEQ ID NO: 170) |
| F-ASO#15 | mixmer | FSE region of ORF1a/b | GGUAUGUGGAAAGGUU |
| | | | (SEQ ID NO: 171) |
| F-ASO#16 | mixmer | FSE region of ORF1a/b | AGUCUGUACCGUCUGC |
| | | | (SEQ ID NO: 172) |
| F-ASO#17 | mixmer | FSE region of ORF1a/b | ACACUUAAAAACAC |
| | | | (SEQ ID NO: 173) |
| F-ASO#18 | mixmer | FSE region of ORF1a/b | ACCCUGUGGGUUUU |
| | | | (SEQ ID NO: 174) |
| F-ASO#19 | mixmer | FSE region of ORF1a/b | CGCUUCCAAGAAAAGG |
| | | | (SEQ ID NO: 175) |
| F-ASO#20 | mixmer | FSE region of ORF1a/b | AAAAACUAAUUGUUGU |
| | | | (SEQ ID NO: 176) |
| F-ASO#21 | mixmer | FSE region of ORF1a/b | GUUUUGCUAAAUUCCU |
| | | | (SEQ ID NO: 177) |
| F-ASO#22 | mixmer | FSE region of ORF1a/b | AAUGAUAAAGUAGCUG |
| | | | (SEQ ID NO: 178) |
| F-ASO#23 | mixmer | FSE region of ORF1a/b | GGCUUUUGACAUCUAC |
| | | | (SEQ ID NO: 179) |
| F-ASO#24 | mixmer | FSE region of ORF1a/b | CUGAUGUCGUAUACAG |
| | | | (SEQ ID NO: 155) |
| F-ASO#25 | mixmer | FSE region of ORF1a/b | GGCACAGGCACUAGUA |
| | | | (SEQ ID NO: 180) |
| F-ASO#26 | mixmer | FSE region of ORF1a/b | CCGUCUUACACCGUGC |
| | | | (SEQ ID NO: 181) |
| F-ASO#27 | mixmer | FSE region of ORF1a/b | GCGGUGUAAGUGCAGC |
| | | | (SEQ ID NO: 182) |
| F-ASO#28 | mixmer | FSE region of ORF1a/b | GUUUUUAAACGGGUUU |
| | | | (SEQ ID NO: 183) |
| F-ASO#29 | mixmer | FSE region of ORF1a/b | CAGCUGAUGCACAAUC |
| | | | (SEQ ID NO: 184) |
| F-ASO#30 | mixmer | FSE region of ORF1a/b | GAACCCAUGCUUCAGU |
| | | | (SEQ ID NO: 185) |
| F-ASO#31 | mixmer | FSE region of ORF1a/b | UUGUGAUCAACUCCGC |
| | | | (SEQ ID NO: 186) |
| F-ASO#32 | mixmer | FSE region of ORF1a/b | AAGGUUAUGGCUGUAG |
| | | | (SEQ ID NO: 187) |
| F-ASO#33 | mixmer | FSE region of ORF1a/b | GUCUGCGGUAUGUGGA |
| | | | (SEQ ID NO: 188) |
| F-ASO#34 | mixmer | FSE region of ORF1a/b | AAACACAGUCUGUACC |
| | | | (SEQ ID NO: 189) |
| F-ASO#35 | mixmer | FSE region of ORF1a/b | GUGGGUUUUACACUUAA |
| | | | (SEQ ID NO: 190) |
| F-ASO#36 | mixmer | FSE region of ORF1a/b | AAACGGGUUUGCGGUG |
| | | | (SEQ ID NO: 166) |
| F-ASO#37 | mixmer | FSE region of ORF1a/b | AUGCACAAUCGUUUUU |
| | | | (SEQ ID NO: 167) |
| F-ASO#38 | mixmer | FSE region of ORF1a/b | UGCUUCAGUCAGCUG |
| | | | (SEQ ID NO: 191) |
| F-ASO#39 | mixmer | FSE region of ORF1a/b | CUCCGCGAACCCA |
| | | | (SEQ ID NO: 192) |
| F-ASO#40 | mixmer | FSE region of ORF1a/b | UUUAAACGGGUUUGCG |
| | | | (SEQ ID NO: 193) |
| F-ASO#41 | mixmer | FSE region of ORF1a/b | CUGAUGCACAAUCGUU |
| | | | (SEQ ID NO: 194) |
| F-ASO#42 | mixmer | FSE region of ORF1a/b | CCCAUGCUUCAGUCAG |
| | | | (SEQ ID NO: 195) |

ASOs targeting SARS-COV-2 viral RNAs or human ACE2 mRNA; LNA = uppercase, DNA = lowercase

TABLE 1
ASOs targeting SARS-COV spike protein_SK

| Oligo-ID | Sequence (5′-3′) | IDT ordering format | Start | Length | Target site |
|---|---|---|---|---|---|
| CRM0497 | GTAAgattaacacaCTG | +G*+T*+A*+A*G*A*T*T*A*A*C*A*C*A* | 21602 | 17 | CAGUGUGUUAAUCUUAC |
| | (SEQ ID NO: 1) | +C*+T*+G | | | (SEQ ID NO: 97) |
| CRM0484 | ATTAacaataagtAGGG | +A*+T*+T*+A*A*C*A*A*T*A*A*G*T*+A | 21909 | 17 | CCCUACUUAUUGUUAAU |
| | (SEQ ID NO: 2) | *+G*+G*+G | | | (SEQ ID NO: 98) |
| CRM0496 | GCTgagagacatatTCA | +G*+C*+T*G*A*G*A*G*A*C*A*T*A*T*+ | 22066 | 17 | UGAAUAUGUCUCUCAGC |
| | (SEQ ID NO: 3) | T*+C*+A | | | (SEQ ID NO: 99) |
| CRM0504 | TGagagagggtcaaGTG | +T*+G*A*G*A*G*A*G*G*G*T*C*A*A*+G | 22437 | 17 | CACUUGACCCUCUCUCA |
| | (SEQ ID NO: 4) | *+T*+G | | | (SEQ ID NO: 100) |
| CRM0488 | CGGAattatatagGAC | +C*+G*+G*+A*A*T*T*A*T*A*T*A*G*+G | 22661 | 16 | GUCCUAUAUAAUUCCG |
| | (SEQ ID NO: 5) | *+A*+C | | | (SEQ ID NO: 101) |
| CRM0489 | GACAttagtaaagcAGA | +G*+A*+C*+A*T*T*A*G*T*A*A*A*G*C* | 22731 | 17 | UCUGCUUUACUAAUGUC |
| | (SEQ ID NO: 6) | +A*+G*+A | | | (SEQ ID NO: 102) |
| CRM0490 | GACtcagtaagaacaCC | +G*+A*+C*T*C*A*G*T*A*A*G*A*A*C*A | 23210 | 17 | GGUGUUCUUACUGAGUC |
| | (SEQ ID NO: 7) | *+C*+C | | | (SEQ ID NO: 103) |
| CRM0501 | TAATgtcaagaatCTCA | +T*+A*+A*+T*G*T*C*A*A*G*A*A*T*+C | 23308 | 17 | UGAGAUUCUUGACAUUA |
| | (SEQ ID NO: 8) | *+T*+C*+A | | | (SEQ ID NO: 104) |
| CRM0500 | GTTAgaagtatttGTTC | +G*+T*+T*+A*G*A*A*G*T*A*T*T*T*+G | 23364 | 17 | GAACAAAUACUUCUAAC |
| | (SEQ ID NO: 9) | *+T*+T*+C | | | (SEQ ID NO: 105) |
| CRM0481 | AGTaggagtaagttGAT | +A*+G*+T*A*G*G*A*G*T*A*A*G*T*T*+ | 23442 | 17 | AUCAACUUACUCCUACU |
| | (SEQ ID NO: 10) | G*+A*+T | | | (SEQ ID NO: 106) |
| CRM0487 | CGAggagaattagtcTG | +C*+G*+A*G*G*A*G*A*A*T*T*A*G*T*C | 23591 | 17 | CAGACUAAUUCUCCUCG |
| | (SEQ ID NO: 11) | *+T*+G | | | (SEQ ID NO: 107) |
| CRM0492 | GAGTtattagagtaaGC | +G*+A*+G*+T*T*A*T*T*A*G*A*G*T*A* | 23678 | 17 | GCUUACUCUAAUAACUC |
| | (SEQ ID NO: 12) | A*+G*+C | | | (SEQ ID NO: 108) |
| CRM0503 | TAgcagcaatatcacCA | +T*+A*G*C*A*G*C*A*A*T*A*T*C*A*C* | 24085 | 17 | UGGUGAUAUUGCUGCUA |
| | (SEQ ID NO: 13) | +C*+A | | | (SEQ ID NO: 109) |
| CRM0502 | TAgcagcaagattagCA | +T*+A*G*C*A*G*C*A*A*G*A*T*T*A*G* | 24625 | 17 | UGCUAAUCUUGCUGCUA |
| | (SEQ ID NO: 14) | +C*+A | | | (SEQ ID NO: 110) |
| CRM0491 | GACTgagggaaggacAT | +G*+A*+C*+T*G*A*G*G*G*A*A*G*G*A* | 24710 | 17 | AUGUCCUUCCCUCAGUC |
| | (SEQ ID NO: 15) | C*+A*+T | | | (SEQ ID NO: 111) |
| CRM0499 | GTCTgtagtaatGATT | +G*+T*+C*+T*G*T*A*G*T*A*A*T*+G*+ | 24901 | 16 | AAUCAUUACUACAGAC |
| | (SEQ ID NO: 16) | A*+T*+T | | | (SEQ ID NO: 112) |
| CRM0493 | GCAaaggatcataAACT | +G*+C*+A*A*A*G*G*A*T*C*A*T*A*+A* | 24970 | 17 | AGUUUAUGAUCCUUUGC |
| | (SEQ ID NO: 17) | +A*+C*+T | | | (SEQ ID NO: 113) |

TABLE 2
hACE2 gapmer ASO designs

| ASO id | Sequence (5′-3′) | MoA | IDT order format (5′-3′) | Start | Length | Target site |
|---|---|---|---|---|---|---|
| CRM0443 | ACACtcatcataacCTA | gapmer | +A*+C*+A*+C*T*C*A*T*C*A*T* | 15572475 | 17 | UAGGUUAUGAUGAGUGU |
| | (SEQ ID NO: 18) | | A*A*C*+C*+T*+A | | | (SEQ ID NO: 114) |
| CRM0444 | ACagtagaaggttaGGT | gapmer | +A*+C*A*G*T*A*G*A*A*G*G*T* | 15584837 | 17 | ACCUAACCUUCUACUGU |
| | (SEQ ID NO: 19) | | T*A*+G*+G*+T | | | (SEQ ID NO: 115) |
| CRM0446 | ACTCgtaagacacATT | gapmer | +A*+C*+T*+C*G*T*A*A*G*A*C* | 15567861 | 16 | AAUGUGUCUUACGAGU |
| | (SEQ ID NO: 20) | | A*C*+A*+T*+T | | | (SEQ ID NO: 116) |
| CRM0449 | AGGGaagaacataagGG | gapmer | +A*+G*+G*+G*A*A*G*A*A*C*A* | 15589483 | 17 | CCCUUAUGUUCUUCCCU |
| | (SEQ ID NO: 21) | | T*A*A*G*+G*+G | | | (SEQ ID NO: 117) |
| CRM0450 | AGTgtaggtttaCCG | gapmer | +A*+G*+T*G*T*A*G*G*T*T*T*A | 15568179 | 15 | CGGUAAACCUACACU |
| | (SEQ ID NO: 22) | | *+C*+C*+G | | | (SEQ ID NO: 118) |
| CRM0451 | ATAatcaattcacTGTC | gapmer | +A*+T*+A*A*T*C*A*A*T*T*C*A | 15570266 | 17 | GACAGUGAAUUGAUUAU |
| | (SEQ ID NO: 23) | | *C*+T*+G*+T*+C | | | (SEQ ID NO: 119) |
| CRM0453 | ATggaaggaggttGGAA | gapmer | +A*+T*G*G*A*A*G*G*A*G*G*T* | 15592939 | 17 | UUCCAACCUCCUUCCAU |
| | (SEQ ID NO: 24) | | T*+G*+G*+A*+A | | | (SEQ ID NO: 120) |
| CRM0454 | ATGgaaggaggttGGAA | gapmer | +A*+T*+G*G*A*A*G*G*A*G*G*T | 15592939 | 17 | UUCCAACCUCCUUCCAU |
| | (SEQ ID NO: 24) | | *T*+G*+G*+A*+A | | | (SEQ ID NO: 120) |
| CRM0456 | ATTGtttatcataCTAC | gapmer | +A*+T*+T*+G*T*T*T*A*T*C*A* | 15597889 | 17 | GUAGUAUGAUAAACAAU |
| | (SEQ ID NO: 25) | | T*A*+C*+T*+A*+C | | | (SEQ ID NO: 121) |
| CRM0457 | CAGAcagataagaTATG | gapmer | +C*+A*+G*+A*C*A*G*A*T*A*A* | 15596050 | 17 | CAUAUCUUAUCUGUCUG |
| | (SEQ ID NO: 26) | | G*A*+T*+A*+T*+G | | | (SEQ ID NO: 122) |
| CRM0458 | CCacattacacaaACTC | gapmer | +C*+C*A*C*A*T*T*A*C*A*C*A* | 15580534 | 17 | GAGUUUGUGUAAUGUGG |
| | (SEQ ID NO: 27) | | A*+A*+C*+T*+C | | | (SEQ ID NO: 123) |
| CRM0459 | CCTtataacaggtCG | gapmer | +C*+C*+T*T*A*T*A*A*C*A*G*G | 15572609 | 15 | CGACCUGUUAUAAGG |
| | (SEQ ID NO: 28) | | *T*+C*+G | | | (SEQ ID NO: 124) |
| CRM0460 | CGAagacagtagaAGGT | gapmer | +C*+G*+A*A*G*A*C*A*G*T*A*G | 15584832 | 17 | ACCUUCUACUGUCUUCG |
| | (SEQ ID NO: 29) | | *A*+A*+G*+G*+T | | | (SEQ ID NO: 125) |
| CRM0462 | CGTAgatagcaaATTA | gapmer | +C*+G*+T*+A*G*A*T*A*G*C*A* | 15570937 | 16 | UAAUUUGCUAUCUACG |
| | (SEQ ID NO: 30) | | A*+A*+T*+T*+A | | | (SEQ ID NO: 126) |
| CRM0463 | CTCaataggaaggTTTG | gapmer | +C*+T*+C*A*A*T*A*G*G*A*A*G | 15583717 | 17 | CAAACCUUCCUAUUGAG |
| | (SEQ ID NO: 31) | | *G*+T*+T*+T*+G | | | (SEQ ID NO: 127) |
| CRM0464 | CTGTgagcaaatacAAA | gapmer | +C*+T*+G*+T*G*A*G*C*A*A*A* | 15561091 | 17 | UUUGUAUUUGCUCACAG |
| | (SEQ ID NO: 32) | | T*A*C*+A*+A*+A | | | (SEQ ID NO: 128) |
| CRM0465 | GAGaaatagtagaaGGG | gapmer | +G*+A*+G*A*A*A*T*A*G*T*A*G | 15578833 | 17 | CCCUUCUACUAUUUCUC |
| | (SEQ ID NO: 33) | | *A*A*+G*+G*+G | | | (SEQ ID NO: 129) |
| CRM0466 | GATAtaggaaggatAGG | gapmer | +G*+A*+T*+A*T*A*G*G*A*A*G* | 15587787 | 17 | CCUAUCCUUCCUAUAUC |
| | (SEQ ID NO: 34) | | G*A*T*+A*+G*+G | | | (SEQ ID NO: 130) |
| CRM0467 | GCggtagtataggaGC | gapmer | +G*+C*G*G*T*A*G*T*A*T*A*G* | 15592789 | 16 | GCUCCUAUACUACCGC |
| | (SEQ ID NO: 35) | | G*A*+G*+C | | | (SEQ ID NO: 131) |
| CRM0468 | GTTggagacagttATAA | gapmer | +G*+T*+T*G*G*A*G*A*C*A*G*T | 15571291 | 17 | UUAUAACUGUCUCCAAC |
| | (SEQ ID NO: 36) | | *T*+A*+T*+A*+A | | | (SEQ ID NO: 132) |
| CRM0469 | GTTTgaaggttcaCGT | gapmer | +G*+T*+T*+T*G*A*A*G*G*T*T* | 15588843 | 16 | ACGUGAACCUUCAAAC |
| | (SEQ ID NO: 37) | | C*A*+C*+G*+T | | | (SEQ ID NO: 133) |
| CRM0470 | TACtcaataggaagGTT | gapmer | +T*+A*+C*T*C*A*A*T*A*G*G*A | 15583715 | 17 | AACCUUCCUAUUGAGUA |
| | (SEQ ID NO: 38) | | *A*G*+G*+T*+T | | | (SEQ ID NO: 134) |
| CRM0472 | TCACttacatattACAC | gapmer | +T*+C*+A*+C*T*T*A*C*A*T*A* | 15567371 | 17 | GUGUAAUAUGUAAGUGA |
| | (SEQ ID NO: 39) | | T*T*+A*+C*+A*+C | | | (SEQ ID NO: 135) |
| CRM0473 | TCgagtacagttGGTT | gapmer | +T*+C*G*A*G*T*A*C*A*G*T*T* | 15571278 | 16 | AACCAACUGUACUCGA |
| | (SEQ ID NO: 40) | | +G*+G*+T*+T | | | (SEQ ID NO: 136) |
| CRM0475 | TGGgagtaaggaacACG | gapmer | +T*+G*+G*G*A*G*T*A*A*G*G*A | 15598357 | 17 | CGUGUUCCUUACUCCCA |
| | (SEQ ID NO: 41) | | *A*C*+A*+C*+G | | | (SEQ ID NO: 137) |
| CRM0476 | TGtagagaagggagGTA | gapmer | +T*+G*T*A*G*A*G*A*A*G*G*G* | 15592904 | 17 | UACCUCCCUUCUCUACA |
| | (SEQ ID NO: 42) | | A*G*+G*+T*+A | | | (SEQ ID NO: 138) |
| CRM0477 | TTGataacacacACGG | gapmer | +T*+T*+G*A*T*A*A*C*A*C*A*C | 15581948 | 16 | CCGUGUGUGUUAUCAA |
| | (SEQ ID NO: 43) | | *+A*+C*+G*+G | | | (SEQ ID NO: 139) |

TABLE 3
SARS-COV-2 5′ leader gapmer ASO designs

| ASO id | Sequence (5′-3′) | MoA | IDT order format (5′-3′) | length | target site |
|---|---|---|---|---|---|
| CRM0505 | GTTcgtttagagAAC | gapmer | +G*+T*+T*/IMe-dC/C*G*T*T*T*A*G*A*G* | 15 | GUUCUCUAAACGAAC |
| | (SEQ ID NO: 44) | | +A*+A*+C | | (SEQ ID NO: 140) |
| CRM0506 | CGTttagagaacAGAT | gapmer | +C*+G*+T*T*T*A*G*A*G*A*A*C*+A*+G*+A | 16 | AUCUGUUCUCUAAACG |
| | (SEQ ID NO: 45) | | *+T | | (SEQ ID NO: 141) |
| CRM0507 | GTTtagagaacagATC | gapmer | +G*+T*+T*T*A*G*A*G*A*A*C*A*G*+A*+T* | 16 | GAUCUGUUCUCUAAAC |
| | (SEQ ID NO: 46) | | +C | | (SEQ ID NO: 142) |
| CRM0508 | GAGaacagatctacAAG | gapmer | +G*+A*+G*A*A*C*A*G*A*T*C*T*A*C*+A*+ | 17 | CUUGUAGAUCUGUUCUC |
| | (SEQ ID NO: 47) | | A*+G | | (SEQ ID NO: 143) |
| CRM0509 | GAAcagatctacAAG | gapmer | +G*+A*+A*C*A*G*A*T*C*T*A*C*+A*+A*+G | 15 | CUUGUAGAUCUGUUC |
| | (SEQ ID NO: 48) | | | | (SEQ ID NO: 144) |
| CRM0510 | CAGatctacaagagATC | gapmer | +C*+A*+G*A*T*C*T*A*C*A*A*G*A*G*+A*+ | 17 | GAUCUCUUGUAGAUCUG |
| | (SEQ ID NO: 49) | | T*+C | | (SEQ ID NO: 145) |
| CRM0511 | GATctacaagagaTCG | gapmer | +G*+A*+T*C*T*A*C*A*A*G*A*G*A*+T*+C* | 16 | CGAUCUCUUGUAGAUC |
| | (SEQ ID NO: 50) | | +G | | (SEQ ID NO: 146) |
| CRM0512 | CAAgagatcgaaaGTT | gapmer | +C*+A*+A*G*A*G*A*T*C*G*A*A*A*+G*+T* | 16 | AACUUUCGAUCUCUUG |
| | (SEQ ID NO: 51) | | +T | | (SEQ ID NO: 147) |
| CRM0513 | GATctacaagagATC | gapmer | +G*+A*+T*C*T*A*C*A*A*G*A*G*+A*+T*+C | 16 | GAUCUCUUGUAGAUC |
| | (SEQ ID NO: 52) | | | | (SEQ ID NO: 148) |
| CRM0514 | GAGatcgaaagttGGT | gapmer | +G*+A*+G*A*T*C*G*A*A*A*G*T*T*+G*+G* | 16 | ACCAACUUUCGAUCUC |
| | (SEQ ID NO: 53) | | +T | | (SEQ ID NO: 149) |
| CRM0515 | GAAagttggttgGTTT | gapmer | +G*+A*+A*A*G*T*T*G*G*T*T*G*+G*+T*+T | 16 | AAACCAACCAACUUUC |
| | (SEQ ID NO: 54) | | *+T | | (SEQ ID NO: 150) |
| CRM0516 | GTTggtttgttacCTG | gapmer | +G*+T*+T*G*G*T*T*T*G*T*T*A*C*+C*+T* | 16 | CAGGUAACAAACCAAC |
| | (SEQ ID NO: 55) | | +G | | (SEQ ID NO: 151) |
| CRM0517 | GGTtggtttgttaCCT | gapmer | +G*+G*+T*T*G*G*T*T*T*G*T*T*A*+C*+C* | 16 | AGGUAACAAACCAACC |
| | (SEQ ID NO: 56) | | +T | | (SEQ ID NO: 152) |
| CRM0518 | GTTtgttacctggGAA | gapmer | +G*+T*+T*T*G*T*T*A*C*C*T*G*G*+G*+A* | 16 | UUCCCAGGUAACAAAC |
| | (SEQ ID NO: 57) | | +A | | (SEQ ID NO: 153) |
| CRM0519 | GTTacctgggaaGGT | gapmer | +G*+T*+T*A*C*C*T*G*G*G*A*A*+G*+G*+T | 15 | ACCUUCCCAGGUAAC |
| | (SEQ ID NO: 58) | | | | (SEQ ID NO: 154) |

TABLE 4
SARS-COV-2 5′ leader mixmer ASO designs

| ASO id | Sequence (5′-3′) | MoA | IDT order format (5′-3′) | length | target site |
|---|---|---|---|---|---|
| CRM0520 | GTtCgTtTAGagAAC | mixmer | +G*+T*T*+C*G*+T*T*+T*A*+G*A*G*+A*+A*+C | 15 | GUUCUCUAAACGAAC |
| | (SEQ ID NO: 44) | | | | (SEQ ID NO: 140) |
| CRM0521 | CGTtTaGaGAacAGAT | mixmer | +C*+G*+T*T*+T*A*+G*A*+G*+A*A*C*+A*+G*+ | 16 | AUCUGUUCUCUAAACG |
| | (SEQ ID NO: 45) | | A*+T | | (SEQ ID NO: 141) |
| CRM0522 | GTtTAgAgAaCagATC | mixmer | +G*+T*T*+T*+A*G*+A*G*+A*A*+C*A*G*+A*+T | 16 | GAUCUGUUCUCUAAAC |
| | (SEQ ID NO: 46) | | *+C | | (SEQ ID NO: 142) |
| CRM0523 | GAgAaCAgAtCtAcAAG | mixmer | +G*+A*G*+A*A*+C*+A*G+*A*T*+C*T*+A*C*+A | 17 | CUUGUAGAUCUGUUCUC |
| | (SEQ ID NO: 47) | | +G*+A* | | (SEQ ID NO: 143) |
| CRM0524 | GAaCaGaTcTAcaAG | mixmer | +G*+A*A*+C*A*+G*A*+T*C*+T*+A*C*A*+A*+G | 15 | CUUGUAGAUCUGUUC |
| | (SEQ ID NO: 48) | | | | (SEQ ID NO: 144) |
| CRM0525 | CAgaTcTAcaAgAgaTC | mixmer | +C*+A*G*A*+T*C*+T*+A*C*A*+A*G*+A*G*A*+ | 17 | GAUCUCUUGUAGAUCUG |
| | (SEQ ID NO: 49) | | T*+C | | (SEQ ID NO: 145) |
| CRM0526 | GATctACaAGaGaTCG | mixmer | +G*+A*+T*C*T*+A*+C*A*+A*+G*A*+G*A*+T*+ | 16 | CGAUCUCUUGUAGAUC |
| | (SEQ ID NO: 50) | | C*+G | | (SEQ ID NO: 146) |
| CRM0527 | CAaGaGaTCgaAAgTT | mixmer | +C*+A*A*+G*A*+G*A*+T*+C*G*A*+A*+A*G*+T | 16 | AACUUUCGAUCUCUUG |
| | (SEQ ID NO: 51) | | *+T | | (SEQ ID NO: 147) |
| CRM0528 | GAtCtACaAgAgAtCG | mixmer | +G*+A*T*+C*T*+A*+C*A*+A*G*+A*G*+A*T*+C | 16 | CGAUCUCUUGUAGAUC |
| | (SEQ ID NO: 50) | | *+G | | (SEQ ID NO: 146) |
| CRM0529 | GAgATcGaAAgTtgGT | mixmer | +G*+A*G*+A*+T*C*+G*A*+A*+A*G*+T*T*G*+G | 16 | ACCAACUUUCGAUCUC |
| | (SEQ ID NO: 53) | | *+T | | (SEQ ID NO: 149) |
| CRM0530 | GAaAGtTgGTtGgTTT | mixmer | +G*+A*A*+A*+G*T*+T*G*+G*+T*T*+G*G*+T*+ | 16 | AAACCAACCAACUUUC |
| | (SEQ ID NO: 54) | | T*+T | | (SEQ ID NO: 150) |
| CRM0531 | GTtgGTtTgTTacCTG | mixmer | +G*+T*T*G*+G*+T*T*+T*G*+T*+T*A*C*+C*+T | 16 | CAGGUAACAAACCAAC |
| | (SEQ ID NO: 55) | | *+G | | (SEQ ID NO: 151) |
| CRM0532 | GGtTGgtTtGtTaCCT | mixmer | +G*+G*T*+T*+G*G*T*+T*T*+G*T*+T*A*+C*+C | 16 | AGGUAACAAACCAACC |
| | (SEQ ID NO: 56) | | *+T | | (SEQ ID NO: 152) |
| CRM0533 | GTtTgTtACcTgggAA | mixmer | +G*+T*T*+T*G*+T*T*+A*+C*C*+T*G*G*G*+A* | 16 | UUCCCAGGUAACAAAC |
| | (SEQ ID NO: 57) | | +A | | (SEQ ID NO: 153) |
| CRM0534 | GTtACcTgGgAAgGT | mixmer | +G*+T*T*+A*+C*C*+T*G*+G*G*+A*+A*G*+G*+ | 15 | ACCUUCCCAGGUAAC |
| | (SEQ ID NO: 58) | | T | | (SEQ ID NO: 154) |

TABLE 5
SARS-COV-2 FSE 3′ stem-loop ASO designs

| ASO id | Sequence (5′-3′) | MoA | IDT order format (5′-3′) | length | Target site |
|---|---|---|---|---|---|
| CRM0606 | CTgTAtACgACatCAG | mixmer | +C*+T*G*+T*+A*T*+A*+C*G*+A*+C*A*T* | 16 | CUGAUGUCGUAUACAG |
| | (SEQ ID NO: 59) | | +C*+A*+G | | (SEQ ID NO: 155) |
| CRM0607 | TGtAGaTGtCAaaAGC | mixmer | +T*+G*T*+A*+G*A*+T*+G*T*+C*+A*A*A* | 16 | GCUUUUGACAUCUACA |
| | (SEQ ID NO: 60) | | +A*+G*+C* | | (SEQ ID NO: 156) |

TABLE 6
SARS-COV-2 FSE mixmer ASO designs

| ASO id | Sequence (5′-3′) | MoA | IDT order format (5′-3′) | length | target site |
|---|---|---|---|---|---|
| CRM0564 | CTtCgTCcTtTtcTTG | mixmer | +C*+T*T*+C*G*+T*+C*C*+T*T*+T*T*C*+T*+T*+G | 16 | CAAGAAAAGGACGAAG |
| | (SEQ ID NO: 61) | | | | (SEQ ID NO: 157) |
| CRM0565 | GAaGCgACaACaaTTA | mixmer | +G*+A*A*+G*+C*G*+A*+C*A*+A*+C*A*A*+T*+T*+A | 16 | UAAUUGUUGUCGCUUC |
| | (SEQ ID NO: 62) | | | | (SEQ ID NO: 158) |
| CRM0566 | GTtTtTaGgAAttTAG | mixmer | +G*+T*T*+T*T*+T*A*+G*G*+A*+A*T*T*+T*+A*+G | 16 | CUAAAUUCCUAAAAAC |
| | (SEQ ID NO: 63) | | | | (SEQ ID NO: 159) |
| CRM0567 | CAaAaCCaGCtAcTTT | mixmer | +C*+A*A*+A*A*+C*+C*A*+G*+C*T*+A*C*+T*+T*+T | 16 | AAAGUAGCUGGUUUUG |
| | (SEQ ID NO: 64) | | | | (SEQ ID NO: 160) |
| CRM0568 | ATCaTtGtAgATgTCA | mixmer | +A*+T*+C*A*+T*T*+G*T*+A*G*+A*+T*G*+T*+C*+A | 16 | UGACAUCUACAAUGAU |
| | (SEQ ID NO: 65) | | | | (SEQ ID NO: 161) |
| CRM0569 | AAaGCcCTgTAtaCGA | mixmer | +A*+A*A*+G*+C*C*+C*+T*G*+T*+A*T*A*+C*+G*+A | 16 | UCGUAUACAGGGCUUU |
| | (SEQ ID NO: 66) | | | | (SEQ ID NO: 162) |
| CRM0570 | CAtCAgTaCTaGtGCC | mixmer | +C*+A*T*+C*+A*G*+T*A*+C*+T*A*+G*T*+G*+C*+C | 16 | GGCACUAGUACUGAUG |
| | (SEQ ID NO: 67) | | | | (SEQ ID NO: 163) |
| CRM0571 | TGtGCcGCaCgGtGTA | mixmer | +T*+G*T*+G*+C*C*+G*+C*A*+C*G*+G*T*+G*+T*+A | 16 | UACACCGUGCGGCACA |
| | (SEQ ID NO: 68) | | | | (SEQ ID NO: 164) |
| CRM0572 | AGaCGgGCtGcAcTTA | mixmer | +A*+G*A*+C*+G*G*+G*+C*T*+G*C*+A*C*+T*+T*+A | 16 | UAAGUGCAGCCCGUCU |
| | (SEQ ID NO: 69) | | | | (SEQ ID NO: 165) |
| CRM0573 | CACcGcAaACcCgTTT | mixmer | +C*+A*+C*C*+G*C*+A*A*+A*+C*C*+C*G*+T*+T*+T | 16 | AAACGGGUUUGCGGUG |
| | (SEQ ID NO: 70) | | | | (SEQ ID NO: 166) |
| CRM0574 | AAaAaCGaTTgTgCAT | mixmer | +A*+A*A*+A*A*+C*+G*A*+T*+T*G*+T*G*+C*+A*+T | 16 | AUGCACAAUCGUUUUU |
| | (SEQ ID NO: 71) | | | | (SEQ ID NO: 167) |
| CRM0575 | CAgCtGaCTgAAgCAT | mixmer | +C*+A*G*+C*T*+G*A*+C*+T*G*+A*+A*G*+C*+A*+T | 16 | AUGCUUCAGUCAGCUG |
| | (SEQ ID NO: 72) | | | | (SEQ ID NO: 168) |
| CRM0576 | GGgTTcGcGgAGtTGA | mixmer | +G*+G*G*+T*+T*C*+G*C*+G*G*+A*+G*T*+T*+G*+A | 16 | UCAACUCCGCGAACCC |
| | (SEQ ID NO: 73) | | | | (SEQ ID NO: 169) |
| CRM0577 | TCaCAaCTaCAgcCAT | mixmer | +T*+C*A*+C*+A*A*+C*+T*A*+C*+A*G*C*+C*+A*+T | 16 | AUGGCUGUAGUUGUGA |
| | (SEQ ID NO: 74) | | | | (SEQ ID NO: 170) |
| CRM0578 | AACcTtTCcACaTaCC | mixmer | +A*+A*+C*C*+T*T*+T*+C*C*+A*+C*A*+T*A*+C*+C | 16 | GGUAUGUGGAAAGGUU |
| | (SEQ ID NO: 75) | | | | (SEQ ID NO: 171) |
| CRM0579 | GCaGaCgGtACaGACT | mixmer | +G*+C*A*+G*A*+C*G*+G*T*+A*+C*A*+G*+A*+C*+T | 16 | AGUCUGUACCGUCUGC |
| | (SEQ ID NO: 76) | | | | (SEQ ID NO: 172) |
| CRM0580 | GTGtTtTtAagTGT | mixmer | +G*+T*+G*T*+T*T*+T*T*+A*A*G*+T*+G*+T | 14 | ACACUUAAAAACAC |
| | (SEQ ID NO: 77) | | | | (SEQ ID NO: 173) |
| CRM0581 | AAAaCcCaCagGGT | mixmer | +A*+A*+A*A*+C*C*+C*A*+C*A*G*+G*+G*+T | 14 | ACCCUGUGGGUUUU |
| | (SEQ ID NO: 78) | | | | (SEQ ID NO: 174) |
| CRM0582 | CCtTtTCtTgGAaGCG | mixmer | +C*+C*T*+T*T*+T*+C*T*+T*G*+G*+A*A*+G*+C*+G | 16 | CGCUUCCAAGAAAAGG |
| | (SEQ ID NO: 79) | | | | (SEQ ID NO: 175) |
| CRM0583 | ACaACaAtTaGTtTTT | mixmer | +A*+C*A*+A*+C*A*+A*T*+T*A*+G*+T*T*+T*+T*+T | 16 | AAAAACUAAUUGUUGU |
| | (SEQ ID NO: 80) | | | | (SEQ ID NO: 176) |
| CRM0584 | AGgAaTtTAgCAaAAC | mixmer | +A*+G*G*+A*A*+T*T*+T*+A*G*+C*+A*A*+A*+A*+C | 16 | GUUUUGCUAAAUUCCU |
| | (SEQ ID NO: 81) | | | | (SEQ ID NO: 177) |
| CRM0585 | CAgCTaCtTtAtcATT | mixmer | +C*+A*G*+C*+T*A*+C*T*+T*T*+A*T*C*+A*+T*+T | 16 | AAUGAUAAAGUAGCUG |
| | (SEQ ID NO: 82) | | | | (SEQ ID NO: 178) |
| CRM0586 | GTaGaTgTcAaAaGCC | mixmer | +G*+T*A*+G*A*+T*G*+T*C*+A*A*+A*A*+G*+C*+C | 16 | GGCUUUUGACAUCUAC |
| | (SEQ ID NO: 83) | | | | (SEQ ID NO: 179) |
| CRM0587 | CTgTaTaCgAcAtCAG | mixmer | +C*+T*G*+T*A*+T*A*+C*G*+A*C*+A*T*+C*+A*+G | 16 | CUGAUGUCGUAUACAG |
| | (SEQ ID NO: 59) | | | | (SEQ ID NO: 155) |
| CRM0588 | TACTAgTgCcTgtGCC | mixmer | +T*+A*C*+T*+A*G*+T*G*+C*C*+T*G*T*+G*+C*+C | 16 | GGCACAGGCACUAGUA |
| | (SEQ ID NO: 84) | | | | (SEQ ID NO: 180) |
| CRM0589 | GCaCgGtGtAAgaCGG | mixmer | +G*+C*A*+C*G*+G*T*+G*T*+A*+A*G*A*+C*+G*+G | 16 | CCGUCUUACACCGUGC |
| | (SEQ ID NO: 85) | | | | (SEQ ID NO: 181) |
| CRM0590 | GCtGcAcTtAcAcCGC | mixmer | +G*+C*T*+G*C*+A*C*+T*T*+A*C*+A*C*+C*+G*+C | 16 | GCGGUGUAAGUGCAGC |
| | (SEQ ID NO: 86) | | | | (SEQ ID NO: 182) |
| CRM0591 | AAaCcCGtTtAAaAAC | mixmer | +A*+A*A*+C*C*+C*+G*T*+T*T*+A*+A*A*+A*+A*+C | 16 | GUUUUUAAACGGGUUU |
| | (SEQ ID NO: 87) | | | | (SEQ ID NO: 183) |
| CRM0592 | GAtTgTgCaTCagCTG | mixmer | +G*+A*T*+T*G*+T*G*+C*A*+T*+C*A*G*+C*+T*+G | 16 | CAGCUGAUGCACAAUC |
| | (SEQ ID NO: 88) | | | | (SEQ ID NO: 184) |
| CRM0593 | ACtGaAGcAtGggTTC | mixmer | +A*+C*T*+G*A*+A*+G*C*+A*T*+G*G*G*+T*+T*+C | 16 | GAACCCAUGCUUCAGU |
| | (SEQ ID NO: 89) | | | | (SEQ ID NO: 185) |
| CRM0594 | GCgGaGtTGaTcaCAA | mixmer | +G*+C*G*+G*A*+G*T*+T*+G*A*+T*C*A*+C*+A*+A | 16 | UUGUGAUCAACUCCGC |
| | (SEQ ID NO: 90) | | | | (SEQ ID NO: 186) |
| CRM0595 | CTaCaGcCaTaAcCTT | mixmer | +C*+T*A*+C*A*+G*C*+C*A*+T*A*+A*C*+C*+T*+T | 16 | AAGGUUAUGGCUGUAG |
| | (SEQ ID NO: 91) | | | | (SEQ ID NO: 187) |
| CRM0596 | TCcAcAtAcCgCaGAC | mixmer | +T*+C*C*+A*C*+A*T*+A*C*+C*G*+C*A*+G*+A*+C | 16 | GUCUGCGGUAUGUGGA |
| | (SEQ ID NO: 92) | | | | (SEQ ID NO: 188) |
| CRM0597 | GGtAcAGaCtGtgTTT | mixmer | +G*+G*T*+A*C*+A*+G*A*+C*T*+G*T*G*+T*+T*+T | 16 | AAACACAGUCUGUACC |
| | (SEQ ID NO: 93) | | | | (SEQ ID NO: 189) |
| CRM0598 | TTaAgTGtAaAaCcCAC | mixmer | +T*+T*A*+A*G*+T*+G*T*+A*A*+A*A*+C*C*+C*+A | 17 | GUGGGUUUUACACUUAA |
| | (SEQ ID NO: 94) | | *+C | | (SEQ ID NO: 190) |

TABLE 7
SARS-COV-2 FSE slippery site mixmer ASO designs

| | | | | | |
|---|---|---|---|---|---|
| CRM0599 | CAcCgCaAAcCcgTTT | mixmer | +C*+A*C*+C*G*+C*A*+A*+A*C*+C*C*G*+T*+T*+T | 16 | AAACGGGUUUGCGGUG |
| | (SEQ ID NO: 70) | | | | (SEQ ID NO: 166) |
| CRM0600 | AAaAaCGaTtGtgCAT | mixmer | +A*+A*A*+A*A*+C*+G*A*+T*T*+G*T*G*+C*+A*+T | 16 | AUGCACAAUCGUUUUU |
| | (SEQ ID NO: 71) | | | | (SEQ ID NO: 167) |
| CRM0601 | CAgCtGaCtGAaGCA | mixmer | +C*+A*G*+C*T*+G*A*+C*T*+G*+A*A*+G*+C*+A | 15 | UGCUUCAGUCAGCUG |
| | (SEQ ID NO: 72) | | | | (SEQ ID NO: 191) |
| CRM0602 | TGgGtTCgCgGAG | mixmer | +T*+G*G*+G*T*+T*+C*G*+C*G*+G*+A*+G | 13 | CUCCGCGAACCCA |
| | (SEQ ID NO: 73) | | | | (SEQ ID NO: 192) |
| CRM0603 | CGcAaAcCCgTttAAA | mixmer | +C*+G*C*+A*A*+A*C*+C*+C*G*+T*T*T*+A*+A*+A | 16 | UUUAAACGGGUUUGCG |
| | (SEQ ID NO: 74) | | | | (SEQ ID NO: 193) |
| CRM0604 | AAcGaTtGtGcAtCAG | mixmer | +A*+A*C*+G*A*+T*T*+G*T*+G*C*+A*T*+C*+A*+G | 16 | CUGAUGCACAAUCGUU |
| | (SEQ ID NO: 95) | | | | (SEQ ID NO: 194) |
| CRM0605 | CTgACtGaAgCatGGG | mixmer | +C*+T*G*+A*+C*T*+G*A*+A*G*+C*A*T*+G*+G*+G | 16 | CCCAUGCUUCAGUCAG |
| | (SEQ ID NO: 96) | | | | (SEQ ID NO: 195) |

1. A method of treating COVID-19 comprising administering to a person in need thereof an antisense oligonucleotide (ASO) targeting SARS-CoV-2 viral RNAs or human ACE2 mRNA.

2. The method of claim 1, wherein the oligonucleotide comprises a modification selected from: locked nucleic acid (LNA) modification; 2′ O-methoxyethyl (MOE) modification; 2′ O-methyl (OME) modification; morpholino phosphorodiamidate (MP) modification; and phosphorodiamidate morpholino oligomers (PMOs) modification.

3. The method of claim 1, wherein the oligonucleotide comprises a locked nucleic acid (LNA) modified antisense oligonucleotide (ASO), the LNA modification comprising a methylene bridge connecting 2′-oxygen and 4′-carbon of ribose.

4. The method of claim 1, wherein the administering step comprises delivering the oligonucleotide by injection or inhalation.

5. The method of claim 1, wherein the administering step comprises delivering the oligonucleotide by nasal administration.

6. The method of claim 1, comprising administering to the person a combination of oligonucleotides targeting SARS-CoV-2 viral RNAs and human ACE2 RNA.

7. The method of claim 1, further comprising detecting a resultant diminution in the person of SARS-CoV-2 presence, expression or activity.

8. The method of claim 1, wherein the oligonucleotide hybridizes to a predetermined target site of a SARS-CoV-2 viral RNA region: 5′ leader, ORF1a, ORF1b, S, ORF3a, E, M, ORF6, ORF7, ORFS, N, ORF10, or 3′ UTR.

9. The method of claim 1, wherein the oligonucleotide hybridizes to a predetermined target site of a SARS-CoV-2 viral RNA region: 5′ leader sequence, Spike coding sequence, 5′ untranslated region (UTR) of ORF1a/b, or frameshift element (FSE) region ORF1a/b.

10. The method of claim 1, wherein the oligonucleotide hybridizes to a predetermined target site of a SARS-CoV-2 viral RNA region: 5′ leader sequence.

11. The method of claim 1, wherein the oligonucleotide(s) comprises sufficient complementarity with a target site of a Table herein to effect hybridization thereto.

12. The method of claim 1, wherein the oligonucleotide comprises a sequence complementary with a target site of a Table herein.

13. The method of claim 1, wherein the oligonucleotide comprises a sequence of an antisense oligonucleotide (ASO) of a Table herein.

14. The method of claim 1, wherein the oligonucleotide comprises a sequence and locked nucleic acid (LNA) pattern of an antisense oligonucleotide (ASO) of a Table herein.

15. The method of claim 1, wherein the oligonucleotide hybridizes to a predetermined target site of a SARS-CoV-2 viral RNA region:

| | | | |
|---|---|---|---|
| ASO | 5′ leader sequence | ASO | 5′UTR regions of ORF1a |
| 5′-ASO#11 | AAACCAACCAACUUUC | U-ASO#05 | GGUUUCGUCCGUGUUG |
| | (SEQ ID NO: 150) | | (SEQ ID NO: 196) |
| 5′-ASO#12 | CAGGUAACAAACCAAC | U-ASO#08 | CGUUGACAGGACACGA |
| | (SEQ ID NO: 151) | | (SEQ ID NO: 197) |
| 5′-ASO#15 | ACCUUCCCAGGUAAC | U-ASO#09 | UAAUAACUAAUUACUGU |
| | (SEQ ID NO: 154) | | (SEQ ID NO: 198) |
| 5′-ASO#26 | AAACCAACCAACUUUC | | |
| | (SEQ ID NO: 150) | | |
| 5′-ASO#27 | CAGGUAACAAACCAAC | | FSE region of ORF1a/b |
| | (SEQ ID NO: 151) | | |
| 5′-ASO#29 | UUCCCAGGUAACAAAC | F-ASO#06 | UCGUAUACAGGGCUUU |
| | (SEQ ID NO: 153) | | (SEQ ID NO: 162) |
| | Spike coding region | F-ASO#23 | GGCUUUUGACAUCUAC |
| | | | (SEQ ID NO: 179) |
| S-ASO#11 | CAGUGUGUUAAUCUUAC | CRM0606 | CUGAUGUCGUAUACAG |
| | (SEQ ID NO: 97) | | (SEQ ID NO: 155) |
| | UGAGAUUCUUGACAUUA | | GCUUUUGACAUCUACA. |
| S-ASO#14 | (SEQ ID NO: 104) | CRM0607 | (SEQ ID NO: 156) |

16. The method of claim 1, wherein the oligonucleotide hybridizes to a predetermined target site of human ACE2 mRNA region:

| | Target site | | Target site |
|---|---|---|---|
| ASO | sequence | ASO | sequence |
| CRM0443 | UAGGUUAUGAUGAGU | CRM0463 | CAAACCUUCCUAUUGAG |
| | GU | | (SEQ ID NO: 127) |
| | (SEQ ID NO: 114) | | |
| CRM0444 | ACCUAACCUUCUACU | CRM0464 | UUUGUAUUUGCUCACAG |
| | GU | | (SEQ ID NO: 128) |
| | (SEQ ID NO: 115) | | |
| CRM0446 | AAUGUGUCUUACGAG | CRM0465 | CCCUUCUACUAUUUCUC |
| | U | | (SEQ ID NO: 129) |
| | (SEQ ID NO: 116) | | |
| CRM0449 | CCCUUAUGUUCUUCC | CRM0466 | CCUAUCCUUCCUAUAUC |
| | CU | | (SEQ ID NO: 130) |
| | (SEQ ID NO: 117) | | |
| CRM0450 | CGGUAAACCUACACU | CRM0467 | GCUCCUAUACUACCGC |
| | (SEQ ID NO: 118) | | (SEQ ID NO: 131) |
| CRM0451 | GACAGUGAAUUGAUU | CRM0468 | UUAUAACUGUCUCCAAC |
| | AU | | (SEQ ID NO: 132) |
| | (SEQ ID NO: 119) | | |
| CRM0453 | UUCCAACCUCCUUCC | CRM0469 | ACGUGAACCUUCAAAC |
| | AU | | (SEQ ID NO: 133) |
| | (SEQ ID NO: 120) | | |
| CRM0454 | UUCCAACCUCCUUCC | CRM0470 | AACCUUCCUAUUGAGUA |
| | AU | | (SEQ ID NO: 134) |
| | (SEQ ID NO: 120) | | |
| CRM0456 | GUAGUAUGAUAAACA | CRM0472 | GUGUAAUAUGUAAGUGA |
| | AU | | (SEQ ID NO: 135) |
| | (SEQ ID NO: 121) | | |
| CRM0457 | CAUAUCUUAUCUGUC | CRM0473 | AACCAACUGUACUCGA |
| | UG | | (SEQ ID NO: 136) |
| | (SEQ ID NO: 122) | | |
| CRM0458 | GAGUUUGUGUAAUGU | CRM0475 | CGUGUUCCUUACUCCCA |
| | GG | | (SEQ ID NO: 137) |
| | (SEQ ID NO: 123) | | |
| CRM0459 | CGACCUGUUAUAAGG | CRM0476 | UACCUCCCUUCUCUACA |
| | (SEQ ID NO: 124) | | (SEQ ID NO: 138) |
| CRM0460 | ACCUUCUACUGUCUU | CRM0477 | CCGUGUGUGUUAUCAA |
| | CG | | (SEQ ID NO: 139) |
| | (SEQ ID NO: 125) | | |
| CRM0462 | UAAUUUGCUAUCUACG | | |
| | (SEQ ID NO: 126) | | |

17. The method of claim 1, wherein the oligonucleotide hybridizes to a predetermined target site of a SARS-CoV-2 viral RNA region and/or comprises a sequence of:

| | | |
|---|---|---|
| ASO#11/CRM0515 | GAAagttggttgGTTT (5′-3′) | 5′ leader target: AAACCAACCAACUUUC |
| | (SEQ ID NO: 54) | (SEQ ID NO: 150) |
| ASO#26/CRM0530 | GAaAGtTgGTtGgTTT(5′-3′) | 5′ leader target: AAACCAACCAACUUUC |
| | (SEQ ID NO: 54) | (SEQ ID NO: 150) |

18. The method of claim 1, wherein the oligonucleotide hybridizes to a predetermined target site of a SARS-CoV-2 viral RNA region and/or comprises a sequence of:

| | | FSE region of |
|---|---|---|
| ASO | Sequence | ORF1a/b target: |
| CRM0606 | CTgTAtACgACatCAG(5′-3′) | CUGAUGUCGUAUACAG |
| | (SEQ ID NO: 59) | (SEQ ID NO: 155) |
| CRM0607 | TGtAGaTGtCAaaAGC(5′-3′) | GCUUUUGACAUCUACA |
| | (SEQ ID NO: 60) | (SEQ ID NO: 156) |

19. The method of claim 1, comprising administering to the person a plurality of antisense oligonucleotides (ASOs), each hybridizing to a different target site of SARS-CoV-2 viral RNAs or human ACE2 mRNA.

20. The method of claim 1, comprising administering to the person a plurality of antisense oligonucleotides (ASOs), each hybridizing to a different target site of SARS-CoV-2 viral RNAs or human ACE2 mRNA,
wherein a first of the antisense oligonucleotides (ASOs) hybridizes to predetermined target site of a SARS-CoV-2 viral RNA region and/or comprises a sequence of:

| | | |
|---|---|---|
| ASO#11/CRM0515 | GAAagttggttgGTTT (5′-3′) | 5′ leader target: AAACCAACCAACUUUC |
| | (SEQ ID NO: 54) | (SEQ ID NO: 150)or |
| ASO#26/CRM0530 | GAaAGtTgGTtGgTTT(5′-3′) | 5′ leader target: AAACCAACCAACUUUC |
| | (SEQ ID NO: 54) | (SEQ ID NO: 150); |

and a second of the antisense oligonucleotides (ASOs) hybridizes to a predetermined target site of a SARS-CoV-2 viral RNA region and/or comprises a sequence of:

| | | FSE region of |
|---|---|---|
| ASO | Sequence | ORF1a/b target: |
| CRM0606 | CTgTAtACgACatCAG(5′-3′) | CUGAUGUCGUAUACAG |
| | (SEQ ID NO: 59) | (SEQ ID NO: 155) |
| CRM0607 | TGtAGaTGtCAaaAGC(5′-3′) | GCUUUUGACAUCUACA |
| | (SEQ ID NO: 60) | (SEQ ID NO: 156) |

21. A pharmaceutical composition, such as an inhaler or nebulizer, configured for the method of treating COVID-19 according to claim 1, and comprising:
(a) a locked nucleic acid antisense oligonucleotide (LNA ASO) targeting SARS-CoV-2 viral RNAs or human ACE2, or
(b) a plurality of locked nucleic acid antisense oligonucleotides (LNA ASOs) each targeting a different site of SARS-CoV-2 viral RNAs or human ACE2,
and a pharmaceutically acceptable excipient, in bulk multidosage, or unit dosage.
